(19) United States
(12) Patent Application Publication
(10) Pub. No.: US 2010/0286160 A1
(43) Pub. Date: Nov. 11, 2010

(54) SUBSTITUTED PIPERAZINES AS CB1 ANTAGONISTS

(75) Inventors: Eric J. Gilbert, Scotch Plains, NJ (US); William J. Greenlee, Teaneck, NJ (US); Sarah Wei Li, Belle Mead, NJ (US); Michael W. Miller, Scotch Plains, NJ (US); Jack D. Scott, Scotch Plains, NJ (US); Adrew Stamford, Chatham Township, NJ (US); Chander Shekher Celly, Colonia, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Intervet Inc.

(21) Appl. No.: 12/665,253

(22) PCT Filed: Jun. 25, 2008
(86) PCT No.: PCT/US08/07917

§ 371 Date: Jun. 14, 2010

Related U.S. Application Data
(60) Provisional application No. 60/946,896, filed on Jun. 28, 2007.

(51) Int. Cl. A61K 31/497; C07D 241/04; C07D 403/12; C07D 241/02; C07D 401/12; C07D 401/14; A61P 9/00

(52) U.S. Cl. 514/252.11; 544/400; 544/372; 544/370; 544/357; 514/254.01; 514/254.05; 514/252.12; 544/360; 514/253.13; 544/364; 514/253.09

(57) ABSTRACT

Compounds of Formula (I): or pharmaceutically acceptable salts, solvates, or esters thereof, are useful in treating diseases or conditions mediated by CB1 receptors, such as metabolic syndrome and obesity, neuroinflammatory disorders, cognitive disorders and psychosis, addiction (e.g., smoking cessation), gastrointestinal disorders, and cardiovascular conditions.

### PRIOR APPLICATIONS

This application claims the benefit of priority to Application No. 60/946,896, filed Jun. 28, 2007, which is incorporated in its entirety by reference.

### BACKGROUND OF THE INVENTION

The CB₁ receptor is one of the most abundant neuromodulatory receptors in the brain, and is expressed at high levels in the hippocampus, cortex, cerebellum, and basal ganglia (e.g., Wilson et al., Science, 2002, vol. 296, 678-682). Selective CB₁ receptor antagonists, for example pyrazole derivatives such as rimonabant (e.g., U.S. Pat. No. 6,432,984), can be used to treat various conditions, such as obesity and metabolic syndrome (e.g., Bensaid et al., Molecular Pharmacology, 2003 vol. 63, no. 4, pp. 908-914; Trillou et al., Am. J. Physiol. Regul. Integr. Comp. Physiol. 2002 vol. 284, R345-R353; Kirkham, Am. J. Physiol. Regul. Integr. Comp. Physiol. 2002 vol. 284, R343-R344), neuroinflammatory disorders (e.g., Adam, et al., Expert Opin. Ther. Patents, 2002, vol. 12, no. 10, 1475-1489; U.S. Pat. No. 6,642,258), cognitive disorders and psychosis (e.g., Adam et al., Expert Opin. Ther. Pat., 2002, vol. 12, pp. 1475-1489), addiction (e.g., smoking cessation; U.S. Patent Publ. 2003/0087933), gastrointestinal disorders (e.g., Lange et al., J. Med. Chem. 2004, vol. 47, 627-643) and cardiovascular conditions (e.g., Porter et al., Pharmacology and Therapeutics, 2001 vol. 90, 45-60; Sanofi-Aventis Publication, Bear Stearns Conference, New York, Sep. 14, 2004, pages 19-24). There now exists extensive pre-clinical and clinical data supporting the use of CB1 receptor antagonists/inverse agonists for the treatment of obesity.

Preparations of marijuana (Cannabis sativa) have been used for over 5000 years for both medicinal and recreational purposes. The major psychoactive ingredient of marijuana has been identified as delta-9-tetrahydrocannabinol (delta-9-THC), one of a member of over 60 related cannabinoid compounds isolated from this plant. It has been demonstrated that delta-9-THC exerts its effects via agonist interaction with cannabinoid (CB) receptors. So far, two cannabinoid receptor subtypes have been characterised (CB₁ and CB₂). The CB₁ receptor subtype is found predominantly in the central nervous system, and to a lesser extent in the peripheral nervous system and various peripheral organs. The CB₂ receptor subtype is found predominantly in lymphoid tissues and cells. To date, three endogenous agonists (endocannabinoids) have been identified which interact with both CB₁ and CB₂ receptors (anandamide, 2-arachidonyl glycerol and noladin ether).

Genetically obese rats and mice exhibit markedly elevated endocannabinoid levels in brain regions associated with ingestive behaviour (Di Marzo et al. 2001 Nature 410: 822-825). Furthermore, increased levels of endocannabinoids are observed upon the fasting of normal, lean animals (Kirkham et al., British Journal of Pharmacology 2002, 136(4) 550-557).

Exogenous application of endocannabinoids leads to the same physiological effects observed with delta-9-THC treatment, including appetite stimulation (Jamshida et al., British Journal of Pharmacology 2001, 134: 1151-1 154), analgesia, hypolocomotion, hypothermia, and catalepsy.

CB₁ (CB₁−/−) and CB₂ (CB₂−/−) receptor knockout mice have been used to elucidate the specific role of the two cannabinoid receptor subtypes. Furthermore, for ligands such as delta-9-THC which act as agonists at both receptors, these mice have allowed identification of which receptor subtype is mediating specific physiological effects. CB₁−/−, but not CB₂−/−, mice are resistant to the behavioural effects of agonists such as delta-9-THC. CB₁−/− animals have also been shown to be resistant to both the body weight gain associated with chronic high fat diet exposure, and the appetite-stimulating effects of acute food deprivation.

These findings suggest a clear role for both endogenous and exogenous cannabinoid receptor agonists in increasing food intake and body weight via selective activation of the CB₁ receptor subtype.

The therapeutic potential for cannabinoid receptor ligands has been extensively reviewed (Exp. Opin. Ther. Pat. 1998, 8, 301-313; Exp. Opin. Ther. Pat. 2000, 10, 1529-1538; Trends in Pharm. Sci. 2000, 2 1, 218-224; Exp. Opin. Ther. Pat. 2002, 12(10), 1475-1489).

At least one compound (SR-14171 6A; Rimonabant) characterised as a CB₁ receptor antagonist/inverse agonist is known to be in clinical trials for the treatment of obesity.

Clinical trials with the CB₁ receptor antagonist rimonabant have also observed an antidiabetic action that exceeds that accounted for by weight loss alone (Scheen A. J., et al., Lancet, 2006 in press). CB₁ receptor mRNA is located on α- and β-cells in the Islets of Langerhans and it has been reported that CB₁ receptor agonists reduce insulin release from pancreatic beta cells in vitro in response to a glucose load (Juan-Pico et al, Cell Calcium, 39, (2006), 155-162). Consistent with this, Bermudez-Siva et al., (Eur J. Pharmacol., 531 (2006), 282-284) have reported that CB₁ receptor agonists increase glucose intolerance following ip injection of a glucose load to rats. This effect was reversed by a CB₁ receptor antagonist that increased glucose tolerance in the test when given alone. Thus, the action of rimonabant may be due to a direct action on the pancreas. It is also possible that CB₁ receptor antagonists affect insulin sensitivity indirectly via an action on adiponectin (Chandran et al., Diabetes care, 26, (2003), 2442-2450) which is elevated by CB₁ receptor antagonists (Cota et al., J Clin Invest., 112 (2003), 423-431; Bensaid et al., Mol. Pharmacol., 63 (2003, 908-914). Indeed, it has been reported that endocannabinoid levels are enhanced in the pancreas and adipose tissue of obese and diabetic mice and in the plasma and adipose tissue of obese or type 2 diabetic patients (Matias et al., J Clin Endocrinol and Metab., 9 1 (2006), 3171-3180) suggesting a possible causal role of elevated cannabinoid tone in the onset of type 2 diabetes.

However, there is still a need for improved cannabinoid agents, particularly selective CB₁ receptor antagonists, with fewer side-effects and improved efficacy.

WO 95/25443, U.S. Pat. No. 5,464,788, and U.S. Pat. No. 5,756,504 describe N-arylpiperazine compounds useful for treating preterm labor, stopping labor, and dysmenorrhea. However, none of the N-aryl piperazines exemplified therein have an aryl and/or heteroaryl substituent at both the 1- and 2-positions of the piperazine ring.

WO 01/02372 and U.S. Published Application No. 2003/0186960 describe cyclized amino acid derivatives for treating or preventing neuronal damage associated with neurological diseases. However, none of the 3-aryl piperazine 2-ones exemplified therein have an aryl and/or heteroaryl substituent at both the 1- and 2-positions of the piperazine ring.

WO 96/01656 describes radiolabelled substituted piperazines useful in pharmacological screening procedures, including labeled N-aryl piperazines. However, none of the N-aryl piperazines exemplified therein have an aryl and/or heteroaryl substituent at both the 1- and 2-positions of the piperazine ring.

U.S. Pat. No. 5,780,480 describes N-aryl piperazines useful as fibrinogen receptor antagonists for inhibiting the binding of fibrinogen to blood platelets, and for inhibiting the aggregation of blood platelets. However, none of the N-aryl piperazines exemplified therein have an aryl and/or heteroaryl substituent at both the 1- and 2-positions of the piperazine ring.

WO 03/008559 describes choline analogs useful for treating conditions or disorders. However, the only substituted piperazine derivative exemplified is N-(2-hydroxyethyl)-N′-(2-pyridylmethyl)-piperazine.

JP 3-200758, JP 4-26683, and JP 4-364175 describe N,N′-diarylpiperazines (i.e., 1,4-diarylpiperazines) prepared by reacting bis(2-hydroxyethyl)arylamines with an amine such as aniline. However, no 1, 2-disubstituted piperazines are exemplified.

WO 97/22597 describes various 1,2,4-trisubstituted piperazine derivatives as tachykinin antagonists for treating tachykinin-mediated diseases such as asthma, bronchitis, rhinitis, cough, expectoration, etc. However, none of the 1,2,4-trisubstituted piperazine derivatives exemplified therein have an aryl and/or heteroaryl substituent at both the 1- and 2-positions of the piperazine ring.

EP 0268222, WO 88/01131, U.S. Pat. No. 4,917,896, and U.S. Pat. No. 5,073,544 describe compositions for enhancing the penetration of active agents through the skin, comprising azacyclohexanes, including N-acyl and N,N′-diacylpiperazines. However, none of the N-acyl or N,N′-diacylpiperazines exemplified therein have an aryl and/or heteroaryl substituent at both the 1- and 2-positions of the piperazine ring.

U.S. Pat. No. 6,528,529 describes compounds, including N,N′-disubstituted piperazines, which are selective for muscarinic acetylcholine receptors and are useful for treating diseases such as Alzheimer's disease. However, none of the N,N′-disubstituted piperazines exemplified therein have an aryl and/or heteroaryl substituent at both the 1- and 2-positions of the piperazine ring.

NL 6603256 describes various biologically active piperazine derivatives. However, none of the piperazine derivatives exemplified therein have a substituted aryl and/or heteroaryl substituent at both the 1- and 2-positions of the piperazine ring.

Wikström et al., J. Med. Chem. 2002, 45, 3280-3285, describe the synthesis of 1,2,3,4,10,14b-hexahydro-6-methoxy-2-methyldibnzo[c,f]pyrazine[1,2-a]azepin. However, none of the piperazine intermediates described therein have a substituted aryl and/or heteroaryl substituent at both the 1- and 2-positions of the piperazine ring.

WO 2007/018460 and WO 2007/018459 describe tricyclic piperidines and piperazine containing compounds, compositions, and methods for their use in treating obesity, psychiatric and neurological disorders. However, none of the compounds disclosed have a substituted aryl and/or heteroaryl substituent at both the 1- and 2-positions of a piperazine ring.

WO 2007/020502 describes pyrrolidone compounds as cannabinoid receptor ligands, in particular CB1 receptor ligands, and their use in treating diseases, conditions, and/or disorders modulated by cannabinoid receptor antagonists. However, none of the compounds disclosed have a substituted aryl and/or heteroaryl substituent at both the 1- and 2-positions of a piperazine ring.

WO 2007/057687 and WO2006/060461 describe piperazine derivatives and their use as CB1 antagonists and in treating various diseases, conditions, and/or disorders modulated by cannabinoid receptor antagonists. However, there remains a need in the art for selective CB1 antagonists having a different functional group substitution pattern around the piperazine ring.

### BRIEF SUMMARY OF THE INVENTION

In its many embodiments, the present invention provides novel substituted piperazine compounds as selective CB₁ receptor antagonists for treating various conditions including, but not limited to metabolic syndrome (e.g., obesity, waist circumference, abdominal girth, lipid profile, and insulin sensitivity), neuroinflammatory disorders, cognitive disorders, psychosis, addictive behavior, gastrointestinal disorders, and cardiovascular conditions.

The selective CB₁ receptor antagonists of the present invention are piperazine derivatives having the structure of Formula (I):

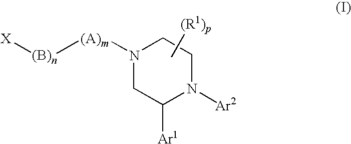

or a pharmaceutically acceptable salt, solvate, isomer, or ester thereof, wherein:
Ar¹ and Ar² are independently aryl or heteroaryl,
   wherein each of Ar¹ and Ar² is substituted with one or more groups independently selected from Y¹;
      with the proviso that when Ar² is pyridine or pyrimidine, a nitrogen of said pyridine or pyrimidine is not in the para position relative to the point of attachment to the piperazine ring;
n and m are independently 0 or 1;
A is selected from the group consisting of —C(O)—, —S(O)₂—, —C(═N—OR²)—, and —(C(R²)₂)_{q}— wherein q is 1, 2, or 3;
B is selected from the group consisting of —N(R²)—, —C(O)—, and —(C(R³)₂)ᵣ— wherein r is 1 or 2,
   with the proviso that when B is —C(O)—, then A is —C(O)— or —(C(R²)₂)_{q}—;
   X is selected from the group consisting of:
   —C(O)N(R⁶)₂, —C(O)-cycloalkyl, —C(O)— heterocycloalkyl, aryl substituted with one or more groups independently selected from —C(O)N(R⁶)₂, heteroaryl substituted with one or more groups independently selected from —C(O)N(R⁶)₂, and benzo-fused cycloalkyl-, wherein the cycloalkyl portion of said benzo-fused cycloalkyl- is substituted with at least one —OH group, and wherein the aryl portion of said benzo-fused cycloalkyl-is unsubstituted or substituted with one or more groups independently selected from Z,
   with the proviso that, when X is —C(O)N(R⁶)₂, —C(O)-cycloalkyl, or —C(O)-heterocycloalkyl, then n=1 and B is —NR²—;
each R¹ is independently selected from the group consisting of alkyl, haloalkyl, -alkylene-NR²R⁵, -alkylene-OR², alkylene-N₃, -alkylene-CN, and alkylene-O—S(O)₂-alkyl; or
two R¹ groups attached to the same ring carbon atom form a carbonyl group;
p is 0, 1, 2, 3, or 4;
each R² is independently H, alkyl, aryl, heteroaryl, cycloalkyl, or heterocycloalkyl,
   wherein each of said aryl heteroaryl, cycloalkyl, and heterocycloalkyl of R² is unsubstituted or optionally substituted with one or more groups independently selected from Y¹;
each R³ is independently selected from the group consisting of H, alkyl, unsubstituted aryl, aryl substituted with one or more Y¹ groups, —OR², -alkylene-O-alkyl, and -alkylene-OH;
each R⁴ is independently selected from the group consisting of H, alkyl, aryl, —C(O)—O-alkyl, —C(O)-alkyl, —C(O)-aryl, —C(O)-heteroaryl, —S(O)₂alkyl, —S(O)₂aryl, —S(O)₂heteroaryl, and —S(O)₂heterocycloalkyl,
   wherein each of said aryl, the aryl portion of said —C(O)-aryl, the aryl portion of said —S(O)₂aryl of R⁴, and the heteroaryl portion of said —C(O)-heteroaryl, and —S(O)₂heteroaryl, is unsubstituted or substituted with one or more groups independently selected from Y¹;
each R⁵ is independently selected from the group consisting of H, alkyl, aryl, —S(O)₂-alkyl, —S(O)₂-cycloalkyl, —S(O)₂-aryl, —S(O)₂-heteroaryl, —S(O)₂-heterocycloalkyl, —C(O)—N(R²)₂, —C(O)-alkyl, —C(O)-cycloalkyl, —C(O)-aryl, —C(O)-heteroaryl, —C(O)-heterocycloalkyl, and -alkylene-OH,
   wherein each of said aryl, the aryl portions of said —S(O)₂-aryl and —C(O)-aryl, and the heteroaryl portions of said —S(O)₂-heteroaryl and said —C(O)-heteroaryl of R⁵ is unsubstituted or substituted with one or more Z groups;
each Y¹ is independently selected from the group consisting of halo, —CN, alkyl, haloalkyl, cycloalkyl, heterocycloalkyl, heterocycloalkenyl, aryl, -alkylene-aryl, heteroaryl, —O-alkyl, —O-haloalkyl, —O-aryl, —O-heteroaryl, —O-cycloalkyl, —O-heterocycloalkyl, —S-aryl, —S-alkyl, —S-haloalkyl, —S-heteroaryl, —S-cycloalkyl, —S-heterocycloalkyl, —S(O)₂-alkyl, —S(O)₂-cycloalkyl, —S(O)₂-heterocycloalkyl, —S(O)₂-aryl, —S(O)₂-heteroaryl, -alkylene-CN, —C(O)-alkyl, —C(O)-aryl, —C(O)-haloalkyl, —C(O)-heteroaryl, —C(O)— cycloalkyl, —C(O)-heterocycloalkyl, —C(O)O-alkyl, —C(O)O-aryl, —C(O)O-haloalkyl, —C(O)O-heteroaryl, —C(O)β-cycloalkyl, —C(O)O-heterocycloalkyl, —N(R²)C(O)-alkyl, —N(R²)C(O)—N(R²)₂, —OH, -alkylene-OH, -alkylene-C(O)—O-alkyl, —O-alkylene-aryl, and —NR²R⁵,
   wherein each of said aryl, each -alkylene-aryl, each heteroaryl, each aryl portion of said —O-aryl, each heteroaryl portion of said —O-heteroaryl, each aryl portion of said —S-aryl, each heteroaryl portion of said —S-heteroaryl, each aryl portion of said —S(O)₂-aryl, each heteroaryl portion of said —S(O)₂-heteroaryl, each aryl portion of said —C(O)-aryl, each heteroaryl portion of said —C(O)-heteroaryl, each aryl portion of said —C(O)O-aryl, and each heteroaryl portion of said —C(O)O-heteroaryl of Y¹ is unsubstituted or substituted with one or more groups Z; or
two groups Y¹ form a —O—CH₂—O— group;
   each R⁶ is independently selected from the group consisting of H, alkyl, haloalkyl, alkoxy, cycloalkyl, heterocycloalkyl, unsubstituted aryl, aryl substituted with one or more groups independently selected from Z, unsubstituted heteroaryl, heteroaryl substituted with one or more groups independently selected from Z, cycloalkyl, -alkylene-OH, -alkylene-O-alkyl, -alkylene-O-aryl, -alkylene-OC(O)-alkyl, -alkylene-OC(O)-aryl, -alkylene-OC(O)-heteroaryl, and alkylene-NR⁴R², or
   two R⁶ groups, together with the nitrogen to which they are attached, form a heteroaryl, heterocycloalkyl, heterocycloalkenyl, or a benzo-fused heterocycloalkyl group; and
each Z is independently selected from the group consisting of alkyl, halo, haloalkyl, —OH, —O-alkyl, and —CN;
   with the proviso that when A is —C(O)—, then each Y¹ on Ar¹ is independently selected from the group consisting of cycloalkyl, benzyl, aryl, —O-haloalkyl, —O-aryl, —O-cycloalkyl, —S-aryl, —S-haloalkyl, —S-cycloalkyl, —S(O)₂-alkyl, —S(O)₂-cycloalkyl, —S(O)₂-aryl, -alkylene-CN, —C(O)-aryl, —C(O)-haloalkyl, —C(O)— cycloalkyl, —C(O)O-aryl, —C(O)O-haloalkyl, —C(O)O-heteroaryl, —C(O)O— cycloalkyl, —C(O)O-heterocycloalkyl, -alkylene-C(O)—O-alkyl, and —O-alkylene-aryl, wherein each benzyl and each aryl portion of Y¹, and each aryl portion and each heteroaryl portion of said —O-aryl, said —S-aryl, said —S(O)₂-aryl, said —C(O)-aryl, said —C(O)O-aryl, —C(O)O-heteroaryl, —C(O)O-heterocycloalkyl, and —O-alkylene-aryl of Y¹, are unsubstituted or substituted with one or more groups independently selected from Z; or two groups Y¹ form a —O—CH₂—O— group.

In another embodiment, the present invention also provides for compositions comprising at least one selective CB₁ receptor antagonist compound of Formula (I), above, or its various embodiments as described herein, or a pharmaceutically acceptable salt, solvate, isomer, or ester thereof, and a pharmaceutically acceptable carrier.

In another embodiment, the present invention also provides for compositions comprising at least on selective CB₁ receptor antagonist compound of Formula (I), or its various embodiments as described herein, or a pharmaceutically acceptable salt, solvate, isomer, or ester thereof, in combination with at least one cholesterol lowering compound or other pharmaceutically active agent, as described herein.

In yet another embodiment, the present invention also provides for a method of treating, reducing, or ameliorating metabolic syndrome, obesity, waist circumference, abdominal girth, lipid profile, insulin sensitivity, neuroinflammatory disorders, cognitive disorders, psychosis, addictive behavior, gastrointestinal disorders, and cardiovascular conditions by administering an effective amount of at least one compound of Formula (I) or its various embodiments as described herein, or a pharmaceutically acceptable salt, solvate, isomer, or ester thereof, to a patient in need thereof.

In yet another embodiment, the present invention also provides for a method of treating vascular conditions, hyperlipidaemia, atherosclerosis, hypercholesterolemia, sitosterolemia, vascular inflammation, metabolic syndrome, stroke, diabetes, obesity and/or reducing the level of sterol(s) in a host in need thereof by administering an effective amount of a composition comprising a combination of at least one compound of Formula (I) or its various embodiments as described herein, or a pharmaceutically acceptable salt, solvate, isomer, or ester thereof, and at least one cholesterol lowering compound.

### DETAILED DESCRIPTION OF THE INVENTION

The selective CB₁ receptor antagonist compounds of the present invention are selective CB₁ receptor antagonists of mammalian CB₁ receptors, preferably human CB₁ receptors, and variants thereof. Mammalian CB₁ receptors also include CB₁ receptors found in rodents, primates, and other mammalian species.

In one embodiment, the selective CB₁ receptor antagonist compounds of the present invention are selective CB₁ receptor antagonists that bind to a CB₁ receptor with a binding affinity (K_{i(CB1)}, measured as described herein) of about 2 μM or less, or about 1 μM or less, or about 400 nM or less, or about 200 nM or less, or about 100 nM or less, or about 10 nM or less. These ranges are inclusive of all values and subranges therebetween.

In one embodiment, the selective CB₁ receptor antagonist compounds of the present invention are selective CB₁ receptor antagonists that have a ratio of CB₁ receptor affinity to CB₂ receptor affinity (K_{i(CB1)}:K_{i(CB1)}, measured as described herein) of about 1:2 or better, or about 1:10 or better, or about 1:25 or better, or about 1:50 or better, or about 1:75 or better, or about 1:90 or better. These ranges are inclusive of all values and subranges therebetween.

Thus, in one embodiment, a selective CB₁ receptor antagonist of the present invention has an affinity for the CB₁ receptor, measured as described herein, of at least 400 nM or less, and a ratio of CB₁ to CB₂ receptor affinity (i.e., (K_{i(CB1)}:K_{i(CB1)}) of at least 1:2 or better. In another embodiment the CB₁ receptor affinity is about 200 nM or less, and the (K_{i(CB1)}:K_{i(CB1)} is about 1:10 or better. In another embodiment the CB₁ affinity is about 100 nM or less, and the (K_{i(CB1)}:K_{i(CB1)} is about 1:25 or better. In another embodiment the CB₁ affinity is about 10 nM or less, and the (K_{i(CB1)}:K_{i(CB1)} is about 1:75 or better. In another embodiment the CB₁ affinity is about 10 nM or less, and the (K_{i(CB1)}:K_{i(CB1)} is about 1:90 or better. These ranges are inclusive of all values and subranges therebetween.

In one embodiment, the present invention provides for a selective CB₁ receptor antagonist compound of Formula (I), or a pharmaceutically acceptable salt, solvate, isomer, or ester thereof, wherein the various substituent groups (i.e., X, Ar¹, Ar², etc.) are as defined hereinabove.

In another embodiment, the present invention relates to a compound of Formula (I), or a pharmaceutically acceptable salt, solvate, isomer, or ester thereof, wherein:
Ar¹ and Ar² are independently (C₆-C₁₀)aryl or (C₂-C₁₀)heteroaryl,
   wherein each of Ar¹ and Ar² is substituted with one or more groups independently selected from Y¹;
      with the proviso that when Ar² is pyridine or pyrimidine, a nitrogen of said pyridine or pyrimidine is not in the para position relative to the point of attachment to the piperazine ring;
n and m are independently 0 or 1;
A is selected from the group consisting of —C(O)—, —S(O)₂—, —C(═N—OR²)—, and —(C(R²)₂)_{q}— wherein q is 1, 2, or 3;
B is selected from the group consisting of —N(R²)—, —C(O)—, and —(C(R³)₂)ᵣ— wherein r is 1 or 2,
   with the proviso that when B is —C(O)—, then A is —C(O)— or —(C(R²)₂)_{q}—;
X is selected from the group consisting of:
   —C(O)N(R⁶)₂, —C(O)—(C₃-C₁₀)cycloalkyl, —C(O)—(C₃-C₁₀)heterocycloalkyl, (C₆-C₁₀)aryl substituted with one or more groups independently selected from —C(O)N(R⁶)₂, (C₂-C₁₀)heteroaryl substituted with one or more groups independently selected from —C(O)N(R⁶)₂, and benzo-fused (C₃-C₁₀)cycloalkyl-, wherein the cycloalkyl portion of said benzo-fused (C₃-C₁₀)cycloalkyl- is substituted with at least one —OH group, and wherein the aryl portion of said benzo-fused (C₃-C₁₀)cycloalkyl- is unsubstituted or substituted with one or more groups independently selected from Z,
   with the proviso that, when X is —C(O)N(R⁶)₂, —C(O)—(C₃-C₁)cycloalkyl, or —C(O)—(C₃-C₁₀)heterocycloalkyl, then n=1 and B is —NR²—;
each R¹ is independently selected from the group consisting of (C₁-C₆)alkyl, (C₁-C₆)haloalkyl, —(C₁-C₆)alkylene-NR²R⁵, —(C₁-C₆)alkylene-OR², —(C₁-C₆)alkylene-N₃, —(C₁-C₆)alkylene-CN, and (C₁-C₆)alkylene-O—S(O)₂—(C₁-C₆)alkyl; or
two R¹ groups attached to the same ring carbon atom form a carbonyl group;
p is 0, 1, 2, 3, or 4;
each R² is independently H, (C₁-C₆)alkyl, (C₆-C₁₀)aryl, (C₂-C₁₀)heteroaryl, (C₃-C₁₀)cycloalkyl, or (C₂-C₁₀)heterocycloalkyl,
   wherein each of said aryl, heteroaryl, cycloalkyl, and heterocycloalkyl of R² is unsubstituted or optionally substituted with one or more groups independently selected from Y¹;
each R³ is independently selected from the group consisting of H, (C₁-C₆)alkyl, unsubstituted (C₆-C₁₀)aryl, (C₆-C₁₀)aryl substituted with one or more Y¹ groups, —OR², —(C₁-C₆)alkylene-O—(C₁-C₆)alkyl, and —(C₁-C₆)alkylene-OH;
each R⁴ is independently selected from the group consisting of H, (C₁-C₆)alkyl, (C₆-C₁₀)aryl, —C(O)—O—(C₁-C₆)alkyl, —C(O)—(C₁-C₆)alkyl, —C(O)—(C₆-C₁₀)aryl, —C(O)—(C₂-C₁₀)heteroaryl, —S(O)₂(C₁-C₆)alkyl, —S(O)₂(C₆-C₁₀)aryl, —S(O)₂(C₂-C₁₀)heteroaryl, and —S(O)₂(C₃-C₁₀)heterocycloalkyl;
   wherein each of said (C₆-C₁₀)aryl, the aryl portion of said —C(O)—(C₆-C₁₀)aryl, the aryl portion of said —S(O)₂(C₆-C₁₀)aryl of R⁴, and the heteroaryl portion of said —C(O)—(C₂-C₁₀)heteroaryl, and —S(O)₂(C₂-C₁₀)heteroaryl, is unsubstituted or substituted with one or more groups independently selected from Y¹;
each R⁵ is independently selected from the group consisting of H, (C₁-C₆)alkyl, (C₆-C₁₀)aryl, —S(O)₂—(C₁-C₆)alkyl, —S(O)₂—(C₃-C₁₀)cycloalkyl, —S(O)₂—(C₆-C₁₀)aryl, —S(O)₂—(C₂-C₁₀)heteroaryl, —S(O)₂—(C₃-C₁₀)heterocycloalkyl, —C(O)—N(R²)₂, —C(O)—(C₁-C₆)alkyl, —C(O)— (C₃-C₁₀)cycloalkyl, —C(O)— (C₆-C₁₀)aryl, —C(O)— (C₆-C₁₀)heteroaryl, —C(O)— (C₃-C₁₀)heterocycloalkyl, and —(C₁-C₆)alkylene-OH,
   wherein each of said aryl, the aryl portions of said —S(O)₂—(C₆-C₁₀)aryl and —C(O)— (C₆-C₁₀)aryl, and the heteroaryl portions of said —S(O)₂—(C₂-C₁₀)heteroaryl and said —C(O)— (C₂-C₁₀)heteroaryl of R⁵ is unsubstituted or substituted with one or more Z groups;
each Y¹ is independently selected from the group consisting of halo, —CN, (C₁-C₆)alkyl, (C₁-C₆)haloalkyl, (C₃-C₁₀)cycloalkyl, (C₂-C₁₀)heterocycloalkyl, (C₂-C₁₀)heterocycloalkenyl, benzyl, (C₆-C₁₀)aryl, (C₂-C₁₀)heteroaryl, —O -(C₁-C₆)alkyl, —O—(C₁-C₆)haloalkyl, —O—(C₆-C₁₀)aryl, —O—(C₂-C₁₀)heteroaryl, —O—(C₃-C₁₀)cycloalkyl, —O—(C₂-C₁₀)heterocycloalkyl, —S—(C₆-C₁₀)aryl, —S—(C₁-C₆)alkyl, —S—(C₁-C₆)haloalkyl, —S—(C₂-C₁₀)heteroaryl, —S—(C₃-C₁₀)cycloalkyl, —S—(C₂-C₁₀)heterocycloalkyl, —S(O)₂—(C₁-C₆)alkyl, —S(O)₂—(C₃-C₁₀)cycloalkyl, —S(O)₂—(C₂-C₁₀)heterocycloalkyl, —S(O)₂—(C₆-C₁₀)aryl, —S(O)₂—(C₂-C₁₀)heteroaryl, —(C₁-C₆)alkylene-CN, —C(O)— (C₁-C₆)alkyl, —C(O)—(C₆-C₁₀)aryl, —C(O)— (C₁-C₆)haloalkyl, —C(O)— (C₂-C₁₀)heteroaryl, —C(O)—(C₃-C₁₀)cycloalkyl, —C(O)— (C₂-C₁₀)heterocycloalkyl, —C(O)O—(C₁-C₆)alkyl, —C(O)O—(C₆-C₁₀)aryl, —C(O)O—(C₁-C₆)haloalkyl, —C(O)O—(C₂-C₁₀)heteroaryl, —C(O)O—(C₃-C₁₀)cycloalkyl, —C(O)O—(C₂-C₁₀)heterocycloalkyl, —N(R²)C(O)—(C₆-C₁₀)alkyl, —N(R²)C(O)—N(R²)₂, —OH, —(C₁-C₆)alkylene-OH, —(C₁-C₆)alkylene-C(O)—O—(C₁-C₆)alkyl, —O—(C₁-C₆)alkylene-(C₆-C₁₀)aryl, and —NR²R⁶,
   wherein each of said benzyl, each aryl, each heteroaryl, each aryl portion of said —O—(C₆-C₁₀)aryl, each heteroaryl portion of said —O—(C₂-C₁₀)heteroaryl, each aryl portion of said —S—(C₆-C₁₀)aryl, each heteroaryl portion of said —S—(C₂-C₁₀)heteroaryl, each aryl portion of said —S(O)₂—(C₆-C₁₀)aryl, each heteroaryl portion of said —S(O)₂—(C₂-C₁₀)heteroaryl, each aryl portion of said —C(O)— (C₆-C₁₀)aryl, each heteroaryl portion of said —C(O)— (C₂-C₁₀)heteroaryl, each aryl portion of said —C(O)O—(C₆-C₁₀)aryl, and each heteroaryl portion of said —C(O)O—(C₂-C₁₀)heteroaryl of Y¹ is unsubstituted or substituted with one or more groups Z; or
two groups Y¹ form a —O—CH₂—O— group;
   each R⁶ is independently selected from the group consisting of H, (C₁-C₆)alkyl, (C₁-C₆)haloalkyl, (C₁-C₆)alkoxy, (C₃-C₁₀)cycloalkyl, (C₃-C₁₀)heterocycloalkyl, unsubstituted (C₆-C₁₀)aryl, (C₆-C₁₀)aryl substituted with one or more groups independently selected from Z, unsubstituted (C₂-C₁₀)heteroaryl, (C₂-C₁₀)heteroaryl substituted with one or more groups independently selected from Z, (C₃-C₁₀)cycloalkyl, —(C₁-C₆)alkylene-OH, —(C₁-C₆)alkylene-O—(C₁-C₆)alkyl, —(C₁-C₆)alkylene-O—(C₆-C₁₀)aryl, —(C₁-C₆)alkylene-OC(O)— (C₁-C₆)alkyl, —(C₁-C₆)alkylene-OC(O)— (C₆-C₁₀)aryl, —(C₁-C₆)alkylene-OC(O)—(C₂-C₁₀)heteroaryl, and (C₁-C₆)alkylene-NR⁴R², or
   two R⁶ groups, together with the nitrogen to which they are attached, form a (C₂-C₁₀)heteroaryl, (C₂-C₁₀)heterocycloalkyl, (C₂-C₁₀)heterocycloalkenyl, or a benzo-fused (C₂-C₁₀)heterocycloalkyl group; and
each Z is independently selected from the group consisting of (C₁-C₆)alkyl, halo, (C₁-C₆)haloalkyl, —OH, —O—(C₁-C₆)alkyl, and —CN;
   with the proviso that when A is —C(O)—, then each Y¹ is independently selected from the group consisting of (C₃-C₁₀)cycloalkyl, benzyl, (C₆-C₁₀)aryl, —O—(C₁-C₆)haloalkyl, —O—(C₆-C₁₀)aryl, —O—(C₃-C₁₀)cycloalkyl, —S—(C₆-C₁₀)aryl, —S—(C₁-C₆)haloalkyl, —S—(C₃-C₁₀)cycloalkyl, —S(O)₂—(C₁-C₆)alkyl, —S(O)₂—(C₃-C₁₀)cycloalkyl, —S(O)₂—(C₆-C₁₀)aryl, —(C₁-C₆)alkylene-CN, —C(O)— (C₆-C₁₀)aryl, —C(O)— (C₁-C₆)haloalkyl, —C(O)—(C₃-C₁₀)cycloalkyl, —C(O)O—(C₆-C₁₀)aryl, —C(O)O—(C₁-C₆)haloalkyl, —C(O)O—(C₂-C₁₀)heteroaryl, —C(O)O—(C₃-C₁₀)cycloalkyl, —C(O)O—(C₂-C₁₀)heterocycloalkyl, —(C₁-C₆)alkylene-C(O)—O—(C₁-C₆)alkyl, and —O—(C₁-C₆)alkylene-(C₆-C₁₀)aryl, wherein each benzyl and each (C₆-C₁₀)aryl portion of Y¹, and each aryl portion and each heteroaryl portion of said —O—(C₆-C₁₀)aryl, said —S—(C₆-C₁₀)aryl, said —S(O)₂—(C₆-C₁₀)aryl, said —C(O)—(C₆-C₁₀)aryl, said —C(O)O—(C₆-C₁₀)aryl, —C(O)O—(C₂-C₁₀)heteroaryl, —C(O)O—(C₂-C₁₀)heterocycloalkyl, and —O—(C₁-C₆)alkylene-(C₆-C₁₀)aryl of Y¹, are unsubstituted or substituted with one or more groups independently selected from Z; or two groups Y¹ form a —O—CH₂—O— group.

In another embodiment, in Formula (I), X is —C(O)N(R⁶)₂. In one such embodiment, at least one R⁶ is H. In another such embodiment, at least one R⁶ is alkyl. In another such embodiment, at least one R⁶ is -alkylene-OH. In another such embodiment, at least one R⁶ is -alkylene-O-alkyl. In another such embodiment two R⁶ groups, together with the nitrogen to which they are attached, form a heteroaryl, heterocycloalkyl, heterocycloalkenyl, or a benzo-fused heterocycloalkyl group.

In another embodiment, in Formula (I), X is —C(O)NH₂.

In another embodiment, in Formula (I), X is —C(O)N(alkyl)₂.

In another embodiment, in Formula (I), X is —C(O)NH(alkyl).

In another embodiment, in Formula (I), X is —C(O)NH(alkylene-OH).

In another embodiment, in Formula (I), X is —C(O)N(alkylene-OH)₂.

In another embodiment, in Formula (I), X is —C(O)NH(alkylene-Oalkyl).

In another embodiment, in Formula (I), X is —C(O)N(alkylene-Oalkyl)₂.

In another embodiment, in Formula (I), X is

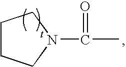

wherein t=0, 1, 2, or 3. In one such embodiment, t=1.

In another embodiment, in Formula (I), X is

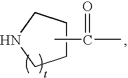

wherein t=0, 1, 2, or 3. In one such embodiment, t=1.

In another embodiment, in Formula (I), X is —C(O)-cycloalkyl. In one such embodiment, said cycloalkyl of X is unsubstituted. In another such embodiment, said cycloalkyl of X is substituted with one or more groups independently selected from Z. In one such embodiment, X is —C(O)-cyclopropyl. In another such embodiment, X is —C(O)-cyclobutyl. In another such embodiment, X is —C(O)-cyclopentyl. In another such embodiment, X is —C(O)-cyclohexyl.

In another embodiment, in Formula (I), X is aryl substituted with one or more groups independently selected from —C(O)N(R⁶)₂. In one such embodiment, said aryl of X is phenyl. In one such embodiment, said aryl of X is naphthyl. In another such embodiment, at least one R⁶ is H. In another such embodiment, at least one R⁶ is alkyl. In another such embodiment, at least one R⁶ is -alkylene-OH. In another such embodiment, at least one R⁶ is -alkylene-O-alkyl. In another such embodiment two R⁶ groups, together with the nitrogen to which they are attached, form a heteroaryl, heterocycloalkyl, heterocycloalkenyl, or a benzo-fused heterocycloalkyl group.

In another embodiment, in Formula (I), X is aryl substituted with at least one group —C(O)NH₂.

In another embodiment, in Formula (I), X is aryl substituted with at least one group independently selected from —C(O)N(alkyl)₂.

In another embodiment, in Formula (I), X is aryl substituted with at least one group independently selected from —C(O)NH(alkyl).

In another embodiment, in Formula (I), X is aryl substituted with at least one group independently selected from —C(O)NH(alkylene-OH).

In another embodiment, in Formula (I), X is aryl substituted with at least one group independently selected from —C(O)N(alkylene-OH)₂.

In another embodiment, in Formula (I), X is aryl substituted with at least one group independently selected from —C(O)NH(alkylene-Oalkyl).

In another embodiment, in Formula (I), X is aryl substituted with at least one group selected from —C(O)N(alkylene-Oalkyl)₂.

In another embodiment, in Formula (I), X is aryl substituted with at least one group independently selected from

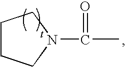

wherein t=0, 1, 2, or 3. In one such embodiment, t=1.

In another embodiment, in Formula (I), X is heteroaryl substituted with one or more groups independently selected from —C(O)N(R⁶)₂. In one such embodiment, at least one R⁶ is H. In another such embodiment, at least one R⁶ is alkyl. In another such embodiment, at least one R⁶ is -alkylene-OH. In another such embodiment, at least one R⁶ is -alkylene-O-alkyl. In another such embodiment two R⁶ groups, together with the nitrogen to which they are attached, form a heteroaryl, heterocycloalkyl, heterocycloalkenyl, or a benzo-fused heterocycloalkyl group.

In another embodiment, in Formula (I), X is heteroaryl substituted with at least one group —C(O)NH₂.

In another embodiment, in Formula (I), X is heteroaryl substituted with at least one group independently selected from —C(O)N(alkyl)₂.

In another embodiment, in Formula (I), X is heteroaryl substituted with at least one group independently selected from —C(O)NH(alkyl).

In another embodiment, in Formula (I), X is heteroaryl substituted with at least one group independently selected from —C(O)NH(alkylene-OH).

In another embodiment, in Formula (I), X is heteroaryl substituted with at least one group independently selected from —C(O)N(alkylene-OH)₂.

In another embodiment, in Formula (I), X is heteroaryl substituted with at least one group independently selected from —C(O)NH(alkylene-Oalkyl).

In another embodiment, in Formula (I), X is heteroaryl substituted with at least one group selected from —C(O)N(alkylene-Oalkyl)₂.

In another embodiment, in Formula (I), X is heteroaryl substituted with at least one group independently selected from

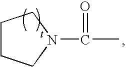

wherein t=0, 1, 2, or 3. In one such embodiment, t=1.

In another embodiment, in Formula (I), X is heteroaryl substituted with at least one group independently selected from

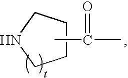

wherein t=0, 1, 2, or 3. In one such embodiment, t=1.

In another embodiment, in Formula (I), X is benzo-fused cycloalkyl-, wherein the cycloalkyl portion of said benzo-fused cycloalkyl- is substituted with at least one —OH groups, and wherein said aryl portion of said benzo-fused cycloalkyl- is unsubstituted. In one such embodiment, said cycloalkyl portion of said benzo-fused cycloalkyl- is substituted with two —OH groups.

In another embodiment, in Formula (I), X is benzo-fused cycloalkyl-, wherein the cycloalkyl portion of said benzo-fused cycloalkyl- is substituted with at least one —OH group, and wherein said aryl portion of said benzo-fused cycloalkyl- is substituted with one or more groups independently selected from halo and CN. In one such embodiment, said cycloalkyl portion of said benzo-fused cycloalkyl- is substituted with two —OH groups. In one such embodiment, Z is fluoro or chloro. In another such embodiment, Z is CN.

In another embodiment, in Formula (I), at least one Y¹ is alkyl. In one such embodiment, at least one Y¹ is (C₁-C₆) alkyl.

In another embodiment, in Formula (I), at least one Y¹ is halo. In one such embodiment, at least one Y¹ is chloro. In one such embodiment, at least one Y¹ is fluoro.

In another embodiment, in Formula (I), at least one Y¹ is —CN.

In another embodiment, in Formula (I), at least one Y¹ is —OH.

In another embodiment, in Formula (I), Ar¹ and Ar² are aryl.

In another embodiment, in Formula (I), Ar¹ is phenyl.

In another embodiment, in Formula (I), Ar² is phenyl.

In another embodiment, in Formula (I), both Ar² and Ar² are phenyl.

In another embodiment, in Formula (I), Ar² is phenyl substituted with two groups independently selected from Y¹.

In another embodiment, in Formula (I), Ar² is phenyl substituted with one Y¹ group in the 4-position and one Y¹ group in the 2-position, relative to the point of attachment to the piperazine ring, which two Y¹ groups may be the same or different, as represented by the moiety below:

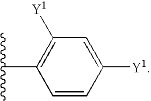

In another embodiment, in Formula (I), Ar¹ is phenyl substituted with one group Y¹ in the 4-position, relative to the point of attachment to the piperazine ring, as represented by the moiety below:

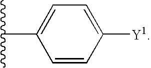

In another embodiment, in Formula (I), Ar¹ is aryl and Ar² is heteroaryl.

In another embodiment, in Formula (I), Ar¹ is phenyl and Ar² is pyridyl.

In another embodiment, in Formula (I), Ar¹ is heteroaryl and Ar² is aryl.

In another embodiment, in Formula (I), Ar¹ is pyridyl and Ar² is phenyl.

In another embodiment, in Formula (I), Ar¹ and Ar² are heteroaryl.

In another embodiment, in Formula (I), Ar¹ is pyridyl.

In another embodiment, in Formula (I), Ar² is pyridyl.

In another embodiment, in Formula (I), both Ar¹ and Ar² are pyridyl.

In another embodiment, in Formula (I), Ar² is pyridyl substituted with two groups independently selected from Y¹.

In another embodiment, in Formula (I), Ar² is pyridyl substituted with one Y¹ group in the 2-position and one Y¹ group in the 4-position, relative to the point of attachment to the piperazine ring, which Y¹ groups may be the same or different.

In another embodiment, in Formula (I), Ar² is:

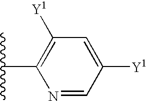

wherein each Y¹ is independently as defined herein.

In another embodiment, in Formula (I), Ar² is substituted with two groups, each independently selected from Y¹.

In another embodiment, in Formula (I), Ar² is substituted with three groups, each independently selected from Y¹.

In another embodiment, in Formula (I), Ar² is substituted with four groups, each independently selected from Y¹.

In another embodiment, in Formula (I), Ar² is substituted with five groups, each independently selected from Y¹.

In another embodiment, in Formula (I), m=0 and n=0.

In another embodiment, in Formula (I), m=0, n=1, and B is —(C(R³)₂)ᵣ—. In one such embodiment, r=1. In another such embodiment each R³ is independently selected from H and -alkylene-OH. In another such embodiment, each R³ is independently selected from H and —(CH₂)—OH. In another such embodiment, each R³ is independently selected from H and —(CH₂)₂—OH. In another such embodiment, each R³ is independently selected from H and —(CH₂)₃—OH.

In another embodiment, in Formula (I), m=0, n=1, and B is —(C(R³)₂)ᵣ—, wherein r=1, and each R³ is independently selected from H and -alkyl. In another such embodiment, each R³ is independently selected from H and methyl. In another such embodiment, each R³ is independently selected from H and ethyl.

In another embodiment, in Formula (I), m=1, n=0, and A is —(C(R²)₂)_{q}—. In one such embodiment, each R² is independently selected from H or alkyl. In another such embodiment, q is 1 and each R² is H. In another such embodiment, q is 2 and each R² is independently selected from H and alkyl.

In another embodiment, in Formula (I), m=1, n=0, and A is —C(O)—.

In another embodiment, in Formula (I), m=1, n=0, and A is —S(O)₂—.

In another embodiment, in Formula (I), m=1, n=1, and A is —(C(R²)₂)_{q}— and B is —(C(R³)₂)ᵣ—. In one such embodiment, q=1 and each R² is H. In one such embodiment, r=1. In another such embodiment, each R³ is independently selected from alkyl and —OR², wherein each R² is independently selected from H or alkyl. In another such embodiment, m=1, n=1, and A is —CH₂—, and B is —C(CH₃)(OH)—. In another such embodiment, m=1, n=1, and A is —CH₂—, and B is —CH(OH)—.

In another embodiment, in Formula (I), m=1, n=1, and A is —C(═N—OR²)—. In one such embodiment, R² is H.

In another embodiment, in Formula (I), m=1, n=1, A is —(C(R²)₂)_{q}— and B is —C(O)—. In one such embodiment, q is 1. In another such embodiment, q is 1 and R² is H.

In another embodiment, in Formula (I), m=1, n=1, A is —C(O)—, and B is —(C(R³)₂)ᵣ—. In one such embodiment, each R³ is independently selected from H, —OH, and alkyl. In one such embodiment, r is 1. In another such embodiment, r is 1 and each R³ is a group independently selected from H and alkyl. In another such embodiment, r=1 and B is selected from —C(OH)(CH₂CH₃)—. —C(OH)(CH₃)—, and —C(OH)H—.

In another embodiment, in Formula (I), m=1, n=1, A is —C(O)—, and B is —N(R⁶)—. In one such embodiment, R⁶ is H.

In another embodiment, in Formula (I), m=1, n=1, A is —(C(R²)₂)_{q}—, and B is —NR²—. In one such embodiment, q is 1 or 2. In another such embodiment, H, alkyl, halo, aryl, and aryl substituted with one or more halo.

In another embodiment, in Formula (I), p=0.

In another embodiment, in Formula (I), p=1, and R¹ is alkyl.

In another embodiment, in Formula (I), p=1, and R¹ is methyl.

In another embodiment, in Formula (I), p=2. In one such embodiment, two groups R¹ are taken together to form a carbonyl group.

In another embodiment, in Formula (I), the present invention relates to compounds, pharmaceutically acceptable salts, solvates, esters, or isomers of the following Formula (IA):

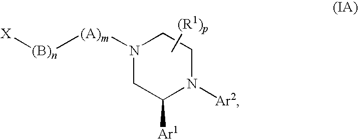

wherein the variables of the formula (e.g., X, B, A, R¹, Ar¹, Ar², n, m, and p) are as defined in Formula (I) above.

In another embodiment of the compounds of Formula (I) of the present invention relates to compounds, pharmaceutically acceptable salts, solvates, esters, or isomers of the following Formula (IB):

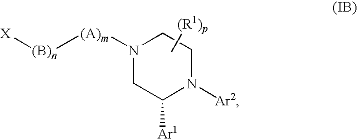

wherein the variables of the formula (e.g., X, B, A, R¹, Ar¹, Ar², n, m, and p) are as defined in Formula (I) above.

In another embodiment of the compounds of Formula (I) of the present invention relates to compounds, pharmaceutically acceptable salts, solvates, esters, or isomers of the following Formula (IC):

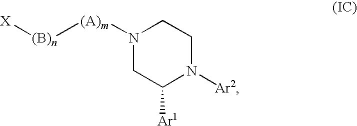

wherein the variables of the formula (e.g., X, B, A, R¹, Ar¹, Ar², n, m, and p) are as defined in Formula (I) above.

In embodiments where n=1 and m=1, then X is attached to B, B is attached to A, and A is attached to the nitrogen of the piperazine ring as shown in the following formula:

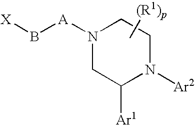

In embodiments where n=0 and m=1, then X is attached directly to A and A is attached to the nitrogen of the piperazine ring as shown in the following formula:

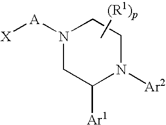

In embodiments where n=1 and m=0, then X is attached to B and B is attached directly to the nitrogen of the piperazine ring as shown in the following formula:

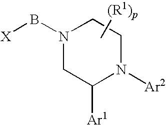

In embodiments where both n and m=0, then X is attached directly to the nitrogen of the piperazine ring as shown in the following formula:

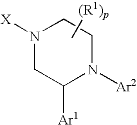

In another embodiment of the compounds of the present invention, or pharmaceutically acceptable salts, solvates, esters, or isomers thereof, is a compound of the Formula (I-D):

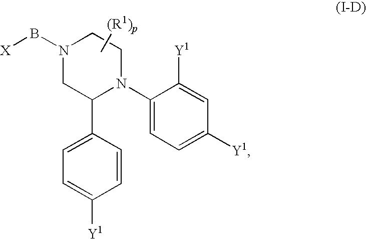

wherein:

B is —(C(R³)₂)ᵣ—, wherein r is 1 or 2;

each R³ is independently selected from H, alkyl, OH, unsubstituted phenyl, and phenyl substituted with one or more groups selected from alkyl, OH, CN, and haloalkyl;

each R¹ is independently selected from alkyl and —C(O)—;

p is 0, 1, or 2;

X is aryl substituted with one or more groups independently selected from —C(O)N(R⁶)₂; and Y¹ and R⁶ are as defined above.

In another embodiment, in Formula (I-D), X is phenyl substituted with one or more groups independently selected from —C(O)N(R⁶)₂. In one such embodiment, at least one R⁶ is H. In another such embodiment, at least one R⁶ is alkyl. In another such embodiment, at least one R⁶ is -alkylene-OH. In another such embodiment, at least one R⁶ is -alkylene-O-alkyl. In another such embodiment two R⁶ groups, together with the nitrogen to which they are attached, form a heteroaryl, heterocycloalkyl, heterocycloalkenyl, or a benzo-fused heterocycloalkyl group.

In another embodiment, in Formula (I-D), X is phenyl substituted with one group —C(O)N(R⁶)₂.

In another embodiment, in Formula (I-D), X is -Ph-C(O)NH₂.

In another embodiment, in Formula (I-D), X is -Ph-C(O)N(alkyl)₂.

In another embodiment, in Formula (I-D), X is -Ph-C(O)NH(alkyl).

In another embodiment, in Formula (I-D), X is -Ph-C(O)NH(alkylene-OH).

In another embodiment, in Formula (I-D), X is -Ph-C(O)N(alkylene-OH)₂.

In another embodiment, in Formula (I-D), X is -Ph-C(O)NH(alkylene-Oalkyl).

In another embodiment, in Formula (I-D), X is -Ph-C(O)N(alkylene-Oalkyl)₂.

In another embodiment, in Formula (I-D), X is

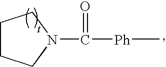

wherein t=0, 1, 2, or 3. In one such embodiment, t=1.

In another embodiment, in Formula (I-D), X is

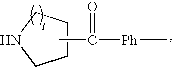

wherein t=0, 1, 2, or 3. In one such embodiment, t=1.

In another embodiment, in Formula (I-D), at least one Y¹ is alkyl. In one such embodiment, at least one Y¹ is (C₁-C₆) alkyl.

In another embodiment, in Formula (I-D), at least one Y¹ is halo. In one such embodiment, at least one Y¹ is chloro. In one such embodiment, at least one Y¹ is fluoro.

In another embodiment, in Formula (I-D), at least one Y¹ is —CN.

In another embodiment, in Formula (I-D), at least one Y¹ is —OH.

In another embodiment of the compounds of the present invention, or pharmaceutically acceptable salts, solvates, esters, or isomers thereof, is a compound of the Formula (I-E):

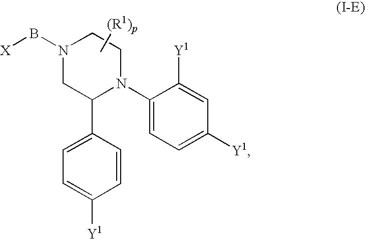

wherein:

B is —(C(R³)₂)ᵣ—, wherein r is 1 or 2;

each R³ is independently selected from H, alkyl, OH, unsubstituted phenyl, and phenyl substituted with one or more groups selected from alkyl, OH, CN, and haloalkyl;

each R¹ is independently selected from alkyl and —C(O)—;

p is 0, 1, or 2;

X is heteroaryl substituted with one or more groups independently selected from —C(O)N(R⁶)₂; and Y¹ and R⁶ are as defined above.

In another embodiment, in Formula (I-E), said heteroaryl of X is pyridinyl.

In another embodiment, in Formula (I-E), said heteroaryl of X is pyrimidinyl.

In another embodiment, in Formula (I-E), said heteroaryl of X is pyrrolyl.

In another embodiment, in Formula (I-E), said heteroaryl of X is imidazolyl.

In another embodiment, in Formula (I-E), at least one R⁶ is H.

In another embodiment, in Formula (I-E), at least one R⁶ is alkyl.

In another embodiment, in Formula (I-E), at least one R⁶ is -alkylene-OH.

In another embodiment, in Formula (I-E), at least one R⁶ is -alkylene-O-alkyl.

In another embodiment, in Formula (I-E), two R⁶ groups, together with the nitrogen to which they are attached, form a heteroaryl, heterocycloalkyl, heterocycloalkenyl, or a benzo-fused heterocycloalkyl group.

In another embodiment, in Formula (I-E), said at least one —C(O)N(R⁶)₂ of X is —C(O)NH₂.

In another embodiment, in Formula (I-E), said at least one —C(O)N(R⁶)₂ of X is —C(O)N(alkyl)₂.

In another embodiment, in Formula (I-E), said at least one —C(O)N(R⁶)₂ of X is —C(O)NH(alkyl).

In another embodiment, in Formula (I-E), said at least one —C(O)N(R⁶)₂ of X is —C(O)NH(alkylene-OH).

In another embodiment, in Formula (I-E), said at least one —C(O)N(R⁶)₂ of X is —C(O)N(alkylene-OH)₂.

In another embodiment, in Formula (I-E), said at least one —C(O)N(R⁶)₂ of X is —C(O)NH(alkylene-Oalkyl).

In another embodiment, in Formula (I-E), said at least one —C(O)N(R⁶)₂ of X is —C(O)N(alkylene-Oalkyl)₂.

In another embodiment, in Formula (I-E), said at least one —C(O)N(R⁶)₂ of X is

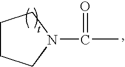

wherein t=0, 1, 2, or 3. In one such embodiment, t=1.

In another embodiment, in Formula (I-E), said at least one —C(O)N(R⁶)₂ of X is

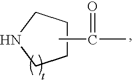

wherein t=0, 1, 2, or 3. In one such embodiment, t=1.

In another embodiment, in Formula (I-E), at least one Y¹ is alkyl. In one such embodiment, at least one Y¹ is (C₁-C₆) alkyl.

In another embodiment, in Formula (I-E), at least one Y¹ is halo. In one such embodiment, at least one Y¹ is chloro. In one such embodiment, at least one Y¹ is fluoro.

In another embodiment, in Formula (I-E), at least one Y¹ is —CN.

In another embodiment, in Formula (I-E), at least one Y¹ is —OH.

In another embodiment of the compounds of the present invention, or pharmaceutically acceptable salts, solvates, esters, or isomers thereof, is a compound of the Formula (I-F):

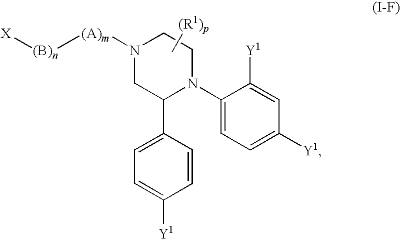

wherein:

m=n=1;

A is —(C(R²)₂)_{q}— wherein q is 1 or 2;

B is —N(R²)—;

each R² is independently selected from H, alkyl, cycloalkyl, unsubstituted aryl, aryl substituted with CN, halo, OH, alkyl, or haloalkyl;

each R¹ is independently selected from alkyl and —C(O)—;

p is 0, 1, or 2;

each Y¹ is independently selected from alkyl, halo, CN, and OH;

X is —C(O)N(R⁶)₂; and R⁶ are as defined above.

In another embodiment, in Formula (I-F), X is —C(O)N(R⁶)₂, wherein at least one R⁶ is H. In another such embodiment, at least one R⁶ is alkyl. In another such embodiment, at least one R⁶ is -alkylene-OH. In another such embodiment, at least one R⁶ is -alkylene-O-alkyl. In another such embodiment two R⁶ groups, together with the nitrogen to which they are attached, form a heteroaryl, heterocycloalkyl, heterocycloalkenyl, or a benzo-fused heterocycloalkyl group.

In another embodiment, in Formula (I-F), X is —C(O)NH₂.

In another embodiment, in Formula (I-F), X is —C(O)N(alkyl)₂.

In another embodiment, in Formula (I-F), X is —C(O)NH(alkyl).

In another embodiment, in Formula (I-F), X is —C(O)NH(alkylene-OH).

In another embodiment, in Formula (I-F), X is —C(O)N(alkylene-OH)₂.

In another embodiment, in Formula (I-F), X is —C(O)NH(alkylene-Oalkyl).

In another embodiment, in Formula (I-F), X is —C(O)N(alkylene-Oalkyl)₂.

In another embodiment, in Formula (I-F), X is

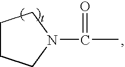

wherein t=0, 1, 2, or 3. In one such embodiment, t=1.

In another embodiment, in Formula (I-F), X is

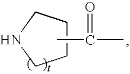

wherein t=0, 1, 2, or 3. In one such embodiment, t=1.

In another embodiment of the compounds of the present invention, or pharmaceutically acceptable salts, solvates, esters, or isomers thereof, is a compound of the Formula (I-G):

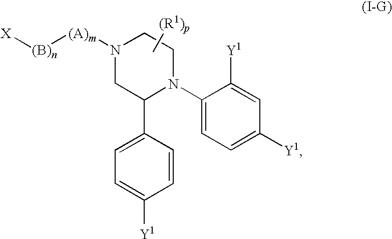

wherein:

m=n=1;

A is —(C(R²)₂)_{q}— wherein q is 1 or 2;

B is —N(R²)—;

each R² is independently selected from H, alkyl, cycloalkyl, unsubstituted aryl, aryl substituted with CN, halo, OH, alkyl, or haloalkyl;

each R¹ is independently selected from alkyl and —C(O)—;

p is 0, 1, or 2;

each Y¹ is independently selected from alkyl, halo, CN, and OH; and

X is —C(O)-cycloalkyl, wherein said cycloalkyl of X is unsubstituted or substituted with one or more groups independently selected from Z, wherein Z is as defined above.

In another embodiment, in Formula (I-G), X is —C(O)-cyclopropyl.

In another embodiment, in Formula (I-G), X is —C(O)-cyclobutyl.

In another embodiment, in Formula (I-G), X is —C(O)-cyclopentyl.

In another embodiment, in Formula (I-G), X is —C(O)-cyclohexyl.

In another embodiment of the compounds of the present invention, or pharmaceutically acceptable salts, solvates, esters, or isomers thereof, is a compound of the Formula (I-H):

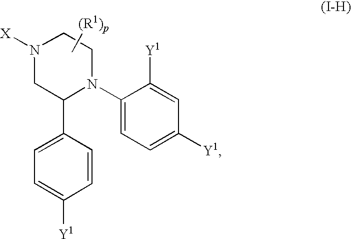

wherein:

each R¹ is independently selected from alkyl and —C(O)—;

p is 0, 1, or 2;

each Y¹ is independently selected from alkyl, halo, CN, and OH; and

X is benzo-fused cycloalkyl-, wherein the cycloalkyl portion of said benzo-fused cycloalkyl- is substituted with at least one —OH group, and wherein said aryl portion of said benzo-fused cycloalkyl- is unsubstituted.

In another embodiment, in Formula (I-H), said benzo-fused cycloalkyl of X is substituted with from one to three —OH groups.

In another embodiment, in Formula (I-H), X is an indanol.

In another embodiment, in Formula (I-H), X is an indandiol.

In another embodiment of the compounds of the present invention, or pharmaceutically acceptable salts, solvates, esters, or isomers thereof, is a compound of the Formula (I-I):

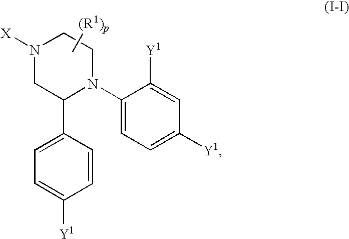

wherein:

each R¹ is independently selected from alkyl and —C(O)—;

p is 0, 1, or 2;

each Y¹ is independently selected from alkyl, halo, CN, and OH; and

X is benzo-fused cycloalkyl-, wherein the cycloalkyl portion of said benzo-fused cycloalkyl- is substituted with at least one —OH group, and wherein said aryl portion of said benzo-fused cycloalkyl- is substituted with halo or —CN.

In another embodiment, in Formula (I-I), said benzo-fused cycloalkyl of X is substituted with from one to three —OH groups.

In another embodiment, in Formula (I-I), X is an indanol, wherein the aryl portion of said indanol is substituted with from one to three groups independently selected from halo.

In another embodiment, in Formula (I-I), X is an indandiol, wherein the aryl portion of said indandiol is substituted with from one to three groups independently selected from halo.

In one embodiment, Ar¹ and Ar² are independently aryl or heteroaryl, wherein each of Ar¹ and Ar² is substituted with one or more groups independently selected from Y¹. Non-limiting examples of said aryl and heteroaryl of Ar¹ and/or Ar² include, for example, phenyl, naphthyl, pyridyl (e.g., 2-, 3-, and 4-pyridyl), pyrimidinyl, quinolyl, thienyl, imidazolyl, furanyl, etc. substituted with one or more (e.g., 1, 2, 3, or 4) Y¹ groups as defined herein.

In one embodiment, A is selected from —C(O)—, —S(O)₂—, —C(═N—OR²)—, and —(C(R²)₂)_{q}— wherein q is 1, 2, or 3. Non-limiting examples of A when A is —(C(R²)₂)_{q}— include, for example, —CH₂—, —CH₂CH₂—, —CH(CH₃)—, —C(CH₃)₂—, —CH₂CH₂CH₂—, —CH(CH₃)CH₂—, —CH₂CH(CH₃)—, —CH(CH₃)—(CH₂)₂—, —(CH₂)₂—CH(CH₃)—, —CH(phenyl)-CH₂—, —CH₂—CH(phenyl)-, —CH(phenyl)-, etc. Non-limiting examples of A when A is —C(═N—OR²)— include —C(═N—OH)—, —C(═N—OCH₃)—, —C(═N—OCH₂CH₃)—, —C(═N—OCH(CH₃)₂)—, —C(═N—OC(CH₃)₃)—, —C(═N—O-phenyl), etc.

In one embodiment, B is selected from —N(R²)—, —C(O)—, and —(C(R³)₂)ᵣ— wherein r is 1, 2, or 3. Non-limiting examples of B when B is —(C(R³)₂)ᵣ—, include, for example, —CH₂—, —CH₂CH₂—, —CH(CH₃)—, —C(CH₃)₂—, —CH(CH(CH₃)₂)—, —CH(CH₂CH(CH₃)₂)—, —CH₂CH₂CH₂—, —CH(CH₃)CH₂—, —CH₂CH(CH₃)—, —CH(CH₃)—(CH₂)₂—, —(CH₂)₂—CH(CH₃)—, —CH(phenyl)-CH₂—, —CH₂—CH(phenyl)-, —CH(phenyl)-, —CH(OH)—, —C(CH₃)(OH)—, —CH(OH)CH₂—, —CH₂CH(OH)—, —CH(OH)CH₂CH(CH₃)—, —CH(CH(OH)(CH₃))—, —CH(CH₃)CH₂CH(OH)—, —CH(CH₂OH)—, —CH(OCH₃)—, —CH(OCH₃)CH₂—, —CH₂CH(OCH₃)—, —CH(OCH₃)CH₂CH(CH₃)—, —CH(CH₃)CH₂CH(OCH₃)—, —CH(CH₂OCH₃)—, —CH(OCH₃)—, —CH(OCH₂CH₃)CH₂—, —CH₂CH(OCH₂CH₃)—, —CH(OCH₂CH₃)CH₂CH(CH₃)—, —CH(CH₃)CH₂CH(OCH₂CH₃)—, —CH(CH₂OCH₂CH₃)—, etc. Non-limiting examples of B when B is —N(R²)— include —NH—, —N(alkyl)-, —N(aryl)-, wherein the terms “alkyl” and “aryl” are as defined herein.

In one embodiment, X is —C(O)N(R⁶)₂. Non-limiting examples of R⁶ when X is —C(O)N(R⁶)₂ include the following. Non-limiting examples of R⁶ when R⁶ is alkyl include any of the examples for alkyl described herein, including methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁶ when R⁶ is halo alkyl include any of the examples for alkyl described herein, including —CF₃, —CHF₂, —CH₂F, —CH₂CF₃, —CF₂CF₃, —CH₂Br, —CH₂Cl, —CCl₃, etc. The “alkyl” portion of R⁶ when R⁶ is alkoxy includes any alkyl group described herein. Non-limiting examples include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁶ when R⁶ is aryl include any of the examples for aryl described herein, including phenyl, naphthyl, etc. When R⁶ is aryl substituted with one or more (e.g., 1, 2, 3, or 4 or more) Y¹ groups, each Y¹ may be independently selected from any of the non-limiting examples for Y¹ described above. When R⁶ is -alkylene-OH, -alkylene-O-alkyl, -alkylene-O-aryl, -alkylene-OC(O)-alkyl, -alkylene-OC(O)-aryl, -alkylene-OC(O)-heteroaryl, and alkylene-N(R₄)₂, non-limiting examples of alkylene and heteroaryl groups include any of those such groups described above. When two R⁶ groups, together with the nitrogen to which they are attached, form a heteroaryl, heterocycloalkyl, heterocycloalkenyl, or a benzo-fused heterocycloalkyl group, non-limiting examples of such heteroaryl, heterocycloalkyl, heterocycloalkenyl, and benzo-fused heterocycloalkyl groups include any of those such groups described above.

In one embodiment, X is —C(O)-cycloalkyl or —C(O)-heterocycloalkyl. Non-limiting examples of X when X is —C(O)-cycloalkyl include —C(O)-cyclopropyl, —C(O)-cyclobutyl, —C(O)-cyclopentyl, —C(O)-cyclohexyl, —C(O)-cycloheptyl, —C(O)-adamantyl, —C(O)-(bicyclo[2.1.1]hexanyl), —C(O)-(bicyclo[2.2.1]heptenyl), —C(O)-(bicyclo[3.1.1]heptenyl), —C(O)-(bicyclo[2.2.2]octenyl), C(O)-(bicyclo[3.2.1]octenyl), etc. Non-limiting examples of X when X is —C(O)-heterocycloalkyl include any heterocycloalkyl groups of —C(O)-heterocycloalkyl described herein.

In one embodiment, X is aryl substituted with one or more groups independently selected from —C(O)N(R⁶)₂. Non-limiting examples include -phenyl-C(O)N(R⁶)₂, -naphthyl—C(O)N(R⁶)₂, etc., wherein —C(O)N(R⁶)₂ is as described herein.

In one embodiment, X is heteroaryl substituted with one or more groups independently selected from —C(O)N(R⁶)₂. Non-limiting examples include heteroaryl include -pyridyl-C(O)N(R⁶)₂, -azaindolyl-C(O)N(R⁶)₂, -benzimidazolyl-C(O)N(R⁶)₂, -benzofuranyl-C(O)N(R⁶)₂, -furanyl-C(O)N(R⁶)₂, -indolyl-C(O)N(R⁶)₂, etc., wherein —C(O)N(R⁶)₂ is as described herein.

In one embodiment, X is benzo-fused cycloalkyl-, wherein the cycloalkyl portion of the benzo-fused cycloalkyl- is substituted with at least one —OH group, and wherein the aryl portion of said benzo-fused cycloalkyl- is unsubstituted or substituted with one or more groups independently selected from Z. Non-limiting examples of benzo-fused cycloalkyl include 1,2,3,4-tetrahydronaphthyl, indanyl, bicyclo[4.2.0]octa-1,3,5-trienyl, etc.

In one embodiment, each R¹ is independently selected from alkyl, haloalkyl, -alkylene-NR²R⁵, -alkylene-OR², alkylene-N₃, and alkylene-O—S(O)₂-alkyl. Non-limiting examples of R¹ when R¹ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R¹ when R¹ is haloalkyl include —CF₃, —CHF₂, —CH₂F, —CH₂CF₃, —CF₂CF₃, —CH₂Br, —CH₂Cl, —CCl₃, etc. When R¹ is alkylene-N₃ or alkylene-O—S(O)₂-alkyl, the alkylene portion thereof can include any of the alkylene groups described herein (e.g., —CH₂—, —CH₂CH₂—, —CH(CH₃)—, —CH₂CH₂CH₂—, —CH(CH₃)CH₂CH₂—, etc. Similarly, the “alkyl” portion of alkylene-O—S(O)₂-alkyl can include any alkyl group described herein (e.g., methyl, ethyl, propyl, butyl, pentyl, etc.) Non-limiting examples of R¹ when R¹ is -alkylene-NR²R⁵ include —CH₂—NR²R⁵, —CH(CH₃)—NR²R⁵, —CH₂CH₂—NR²R⁵, —CH₂CH₂CH₂—NR²R⁵₂, —CH(CH₃)CH₂CH₂—N NR²R⁵, etc., wherein each R² and each R⁵ is independently defined as described herein. For example, the “—NR²R⁵” portion of -alkylene-N NR²R⁵ of R¹ can be —NH₂, —N(CH₃)₂, —NH(CH₃), —NH(phenyl), —N(phenyl)₂, —NH—S(O)₂—CH₃, —NH—S(O)₂-cyclopropyl, —NH—C(O)—NH₂, —NH—C(O)—N(CH₃)₂, —NH—C(O)—CH₃, —NH—CH₂CH₂—OH, etc. Non-limiting examples of R¹ when R¹ is -alkylene-OR² include —CH₂—OR², —CH(CH₃)—OR², —CH₂CH₂—OR², —CH(OR²)CH₂CH(CH₃)₂, —CH(CH₃)CH₂CH₂—OR², wherein R² is defined as described herein. For example, the “—OR²” portion of said -alkylene-OR² of R¹ can be —OH, —OCH₃, —OCH₂CH₃, —OCH(CH₃)₂, —O-phenyl. Alternatively, two R¹ groups attached to the same ring carbon atom can form a carbonyl group, for example as shown below:

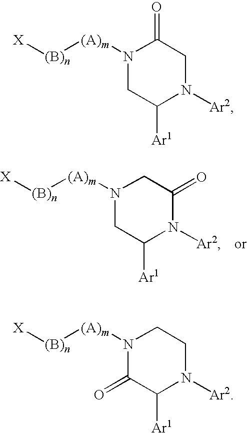

In one embodiment, each R² is independently selected from H, alkyl, aryl, heteroaryl, cycloalkyl, and heterocycloalkyl. Non-limiting examples of R² when R² is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R² when R² is aryl include phenyl, naphthyl, etc. Non-limiting examples of R² when R² is heteroaryl include heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc. Non-limiting examples of R² when R² is cycloalkyl include cycloalkyl include cyclopropyl, cyclobutyl, cyclopentyl, cyclohexyl, adamantyl, norbornyl, etc. Non-limiting examples of R² when R² is heterocycloalkyl include heterocycloalkyl include morpholinyl, piperazinyl, piperidinyl, pyrrolidinyl, tetrahydrofuranyl, tetrahydrothiophenyl, tetrahydropyranyl, azetidinyl, etc., wherein each said aryl, heteroaryl, cycloalkyl, and heterocycloalkyl may be unsubstituted or substituted with one or more groups independently selected from Y¹, as defined herein.

In one embodiment, each R³ is independently selected from H, alkyl, unsubstituted aryl, aryl substituted with one or more Y¹ groups, —OR², -alkylene-O-alkyl, and -alkylene-OH. Non-limiting examples of R³ when R³ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R³ when R³ is aryl include phenyl, naphthyl, etc., wherein said aryl may be unsubstituted or substituted with one or more groups selected from Y¹ groups as defined herein. Non-limiting examples of R³ when R³ is —OR² include —OH, —OCH₃, —OCH₂CH₃, —OCH(CH₃)₂, —O-phenyl, etc. Non-limiting examples of R³ when R³ is -alkylene-O-alkyl include —O—CH₂—O—CH₃, —O—CH₂CH₂—O—C(CH₃)₃, —O—CH(CH₃)—O—CH₃, —O—CH₂CH₂—O—CH₃, —O—CH₂CH₂—O—CH₂CH₃, —O—CH(OCH₃)CH₂CH(CH₃)₂, —O—CH(CH₃)CH₂CH₂—O—CH₃, —O—CH₂CH₂—O—CH₂CH₃, etc. Non-limiting examples of R³ when R³ is -alkylene-OH include —CH₂—OH, —CH₂CH₂—OH, —CH₂CH₂CH₂—OH, —CH(OH)CH₃, —CH₂CH(OH)CH₃, etc.

In one embodiment, each R⁴ is independently selected from H, alkyl, aryl, —C(O)—O-alkyl, —C(O)-alkyl, —C(O)-aryl, —C(O)-heteroaryl, —S(O)₂alkyl, —S(O)₂aryl, —S(O)₂heteroaryl, and —S(O)₂heterocycloalkyl. Non-limiting examples of R⁴ when R⁴ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁴ when R⁴ is aryl include phenyl, naphthyl, etc., wherein said aryl may be unsubstituted or substituted with one or more Y¹ groups as defined herein. Non-limiting examples of R⁴ when R⁴ is —C(O)—O-alkyl include —C(O)—O—CH₃, —C(O)—O—CH₂CH₃, —C(O)—O—CH₂CH₂CH₃, —C(O)—O—CH(CH₃)₂, —C(O)—O—CH₂CH₂CH₂CH₃, —C(O)—O—CH₂CH(CH₃)₂, —C(O)—O—CH(CH₃)CH₂CH₃, —C(O)—O—C(CH₃)₃, —C(O)—O—CH₂CH₂CH₂CH₂CH₃, —C(O)—O—CH₂CH(CH₃)CH₂CH₃, —C(O)—O—CH₂CH₂CH(CH₃)₂, —C(O)—O—CH₂CH₂CH₂CH₂CH₂CH₃, —C(O)—O—CH(CH₃)CH₂CH₂CH₂CH₃, —C(O)—O—CH₂CH(CH₃)CH₂CH₂CH₃, —C(O)—O—CH₂CH₂CH(CH₃)CH₂CH₃, —C(O)—O—CH₂CH₂CH₂CH(CH₃)₂, etc. Non-limiting examples of R⁴ when R⁴ is —C(O)-alkyl include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH₂CH(CH₃)₂, —C(O)—CH(CH₃)CH₂CH₃, —C(O)—C(CH₃)₃, —C(O)—CH₂CH₂CH₂CH₂CH₃, —C(O)—CH₂CH(CH₃)CH₂CH₃, —C(O)—CH₂CH₂CH(CH₃)₂, —C(O)—CH₂CH₂CH₂CH₂CH₂CH₃, —C(O)—CH(CH₃)CH₂CH₂CH₂CH₃, —C(O)—CH₂CH(CH₃)CH₂CH₂CH₃, —C(O)—CH₂CH₂CH(CH₃)CH₂CH₃, —C(O)—CH₂CH₂CH₂CH(CH₃)₂, etc. Non-limiting examples of R⁴ when R⁴ is —C(O)-aryl include —C(O)-phenyl, —C(O)-naphthyl, etc., optionally substituted with one or more groups selected from Y¹. Non-limiting examples of R⁴ when R⁴ is —S(O)₂aryl include —S(O)₂-phenyl, —S(O)₂-naphthyl, etc., optionally substituted with one or more groups selected from Y¹.

In one embodiment, each R⁵ is independently selected from H, alkyl, aryl, —S(O)₂-alkyl, —S(O)₂-cycloalkyl, —S(O)₂-aryl, —S(O)₂-heteroaryl, —S(O)₂-heterocycloalkyl, —C(O)—N(R²)₂, —C(O)-alkyl, —C(O)-cycloalkyl, —C(O)-aryl, —C(O)-heteroaryl, —C(O)-heterocycloalkyl, and -alkylene-OH. Non-limiting examples of R⁵ when R⁵ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of R⁵ when R⁵ is aryl include phenyl, naphthyl, etc., wherein said aryl may be unsubstituted or substituted with one or more Z groups as defined herein. Non-limiting examples of R⁵ when R⁵ is —S(O)₂-alkyl include —S(O)₂—CH₃, —S(O)₂—CH₂CH₃, —S(O)₂—CH₂CH₂CH₃, —S(O)₂—CH(CH₃)₂, —S(O)₂—CH₂CH₂CH₂CH₃, —S(O)₂—CH₂CH(CH₃)₂, —S(O)₂—CH(CH₃)CH₂CH₃, —S(O)₂—C(CH₃)₃, —S(O)₂—CH₂CH₂CH₂CH₂CH₃, —S(O)₂—CH₂CH(CH₃)CH₂CH₃, —S(O)₂—CH₂CH₂CH(CH₃)₂, —S(O)₂—CH₂CH₂CH₂CH₂CH₂CH₃, —S(O)₂—CH(CH₃)CH₂CH₂CH₂CH₃, —S(O)₂—CH₂CH(CH₃)CH₂CH₂CH₃, —S(O)₂—CH₂CH₂CH(CH₃)CH₂CH₃, —S(O)₂—CH₂CH₂CH₂CH(CH₃)₂, etc. Non-limiting examples of R⁵ when R⁵ is —S(O)₂-cycloalkyl include —S(O)₂-cyclopropyl, —S(O)₂-cyclobutyl, —S(O)₂-cyclopentyl, —S(O)₂-cyclohexyl, —S(O)₂-adamantyl, —S(O)₂-norbornyl, —S(O)₂-decalyl, etc. Non-limiting examples of R⁵ when R⁵ is —C(O)—N(R²)₂ include —C(O)—NH₂, —C(O)—NH(alkyl), —C(O)—N(alkyl)₂, —C(O)—NH(aryl), —C(O)—N(alkyl)(aryl), —C(O)—N(aryl)₂, wherein the terms “aryl” and “alkyl” are as defined above, and said “aryl” may be unsubstituted or substituted with one or more Y¹ groups as defined herein. Non-limiting examples of R⁵ when R⁵ is —C(O)-alkyl include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH₂CH(CH₃)₂, —C(O)—CH(CH₃)CH₂CH₃, —C(O)—C(CH₃)₃, —C(O)—CH₂CH₂CH₂CH₂CH₃, —C(O)—CH₂CH(CH₃)CH₂CH₃, —C(O)—CH₂CH₂CH(CH₃)₂, —C(O)—CH₂CH₂CH₂CH₂CH₂CH₃, —C(O)—CH(CH₃)CH₂CH₂CH₂CH₃, —C(O)—CH₂CH(CH₃)CH₂CH₂CH₃, —C(O)—CH₂CH₂CH(CH₃)CH₂CH₃, —C(O)—CH₂CH₂CH₂CH(CH₃)₂, etc. Non-limiting examples of R⁵ when R⁵ is -alkylene-OH include —CH₂—OH, —CH₂CH₂—OH, —CH₂CH₂CH₂—OH, —CH(OH)CH₃, —CH₂CH(OH)CH₃, etc. Non-limiting examples of R⁵ when R⁵ is —S(O)₂aryl include —S(O)₂-phenyl, —S(O)₂-naphthyl, etc., optionally substituted with one or more Y¹ groups.

In one embodiment, each Y¹ is independently selected from the group consisting of halo, —CN, alkyl, haloalkyl, cycloalkyl, heterocycloalkyl, heterocycloalkenyl, aryl, -alkylene-aryl, heteroaryl, —O-alkyl, —O-haloalkyl, —O-aryl, —O-heteroaryl, —O-cycloalkyl, —O-heterocycloalkyl, —S-aryl, —S-alkyl, —S-haloalkyl, —S-heteroaryl, —S-cycloalkyl, —S-heterocycloalkyl, —S(O)₂-alkyl, —S(O)₂-cycloalkyl, —S(O)₂-heterocycloalkyl, —S(O)₂-aryl, —S(O)₂-heteroaryl, -alkylene-CN, —C(O)-alkyl, —C(O)-aryl, —C(O)-haloalkyl, —C(O)-heteroaryl, —C(O)— cycloalkyl, —C(O)-heterocycloalkyl, —C(O)O-alkyl, —C(O)O-aryl, —C(O)O-haloalkyl, —C(O)O-heteroaryl, —C(O)O— cycloalkyl, —C(O)O-heterocycloalkyl, —N(R²)C(O)-alkyl, —N(R²)C(O)—N(R²)₂, —OH, -alkylene-OH, -alkylene-C(O)—O-alkyl, —O-alkylene-aryl, and —NR²R⁵, wherein each benzyl, each aryl, each heteroaryl, each aryl portion of said —O-aryl, each heteroaryl portion of said —O-heteroaryl, each aryl portion of said —S-aryl, each heteroaryl portion of said —S-heteroaryl, each aryl portion of said —S(O)₂-aryl, each heteroaryl portion of said —S(O)₂-heteroaryl, each aryl portion of said —C(O)-aryl, each heteroaryl portion of said —C(O)-heteroaryl, each aryl portion of said —C(O)O-aryl, and each heteroaryl portion of said —C(O)O-heteroaryl of Y¹ are unsubstituted or substituted with one or more groups Z; or two groups Y¹ form a —O—CH₂—O— group.

Non-limiting examples of Y¹ when Y¹ is alkyl include methyl, ethyl, n-propyl, iso-propyl, n-butyl, iso-butyl, sec-butyl, tert-butyl, n-pentyl, iso-pentyl, neo-pentyl, n-hexyl, iso-hexyl, etc. Non-limiting examples of Y¹ when Y¹ is cycloalkyl include cyclopropyl, cyclobutyl, cyclopentyl, cyclohexyl, adamantyl, norbornyl, etc. Non-limiting examples of Y¹ when Y¹ is heterocycloalkyl include morpholinyl, piperazinyl, piperidinyl, pyrrolidinyl, tetrahydrofuranyl, tetrahydrothiophenyl, tetrahydropyranyl, azetidinyl, etc. Non-limiting examples of Y¹ when Y¹ is heterocycloalkenyl include 2H-benzo[1,4]oxazinyl, 4H-chromenyl, 4H-chromenyl, 3H-indolyl, 1H-isoindolyl, 4H-benzo[1,4]oxazinyl, etc. Non-limiting examples of Y¹ when Y¹ is halo include chloro, bromo, and iodo. Non-limiting examples of Y¹ when Y¹ is haloalkyl include —CF₃, —CHF₂, —CH₂F, —CH₂CF₃, —CF₂CF₃, —CH₂Br, —CH₂Cl, —CCl₃, etc. Non-limiting examples of Y¹ when Y¹ is -alkylene-aryl include benzyl, -ethylene-phenyl, -propylene-phenyl, -methylene-naphthyl, and -ethylene-naphthyl, etc. Non-limiting examples of Y¹ when Y¹ is aryl include phenyl, naphthyl, etc. Non-limiting examples of Y¹ when Y¹ is heteroaryl include azaindolyl, benzimidazolyl, benzofuranyl, furanyl, 2-pyridinyl, 3-pyridinyl, 4-pyridinyl, furazanyl, indolyl, quinolyl, isoquinolyl, phthalazinyl, pyrazinyl, pyridazinyl, pyrimidyl, pyrrolyl, quinoxalinyl, thiophenyl, isoxazolyl, triazolyl, thiazolyl, indazolyl, thiadiazolyl, imidazolyl, benzo[b]thiophenyl, tetrazolyl, pyrazolyl, etc. Non-limiting examples of Y¹ when Y¹ is —O-aryl include —O-phenyl, —O-naphthyl, etc. Non-limiting examples of Y¹ when Y¹ is —S-aryl include —S-phenyl, —S-naphthyl, etc. Non-limiting examples of Y¹ when Y¹ is —S(O)₂-alkyl include —S(O)₂—CH₃, —S(O)₂—CH₂CH₃, —S(O)₂—CH₂CH₂CH₃, —S(O)₂—CH(CH₃)₂, —S(O)₂—CH₂CH₂CH₂CH₃, —S(O)₂—CH₂CH(CH₃)₂, —S(O)₂—CH(CH₃)CH₂CH₃, —S(O)₂—C(CH₃)₃, —S(O)₂—CH₂CH₂CH₂CH₂CH₃, —S(O)₂—CH₂CH(CH₃)CH₂CH₃, —S(O)₂—CH₂CH₂CH(CH₃)₂, —S(O)₂—CH₂CH₂CH₂CH₂CH₂CH₃, —S(O)₂—CH(CH₃)CH₂CH₂CH₂CH₃, —S(O)₂—CH₂CH(CH₃)CH₂CH₂CH₃, —S(O)₂—CH₂CH₂CH(CH₃)CH₂CH₃, —S(O)₂—CH₂CH₂CH₂CH(CH₃)₂, etc. Non-limiting examples of Y¹ when Y¹ is —S(O)₂-cycloalkyl include —S(O)₂-cyclopropyl, —S(O)₂-cyclobutyl, —S(O)₂-cyclopentyl, —S(O)₂-cyclohexyl, —S(O)₂-adamantyl, —S(O)₂-norbornyl, etc. Non-limiting examples of Y¹ when Y¹ is —S(O)₂-aryl include —S(O)₂-phenyl, —S(O)₂-naphthyl, etc. Non-limiting examples of Y¹ when Y¹ is -alkylene-CN include —O—CH₂—CN, —O—CH₂CH₂—CN, —CH₂CH₂CH₂CN, —O—CH(CH₃)—CN, —O—CH(CN)CH₂CH(CH₃)₂, —O—CH(CH₃)CH₂CH₂—CN, etc. Non-limiting examples of Y¹ when Y¹ is —C(O)-alkyl include —C(O)—CH₃, —C(O)—CH₂CH₃, —C(O)—CH₂CH₂CH₃, —C(O)—CH(CH₃)₂, —C(O)—CH₂CH₂CH₂CH₃, —C(O)—CH₂CH(CH₃)₂, —C(O)—CH(CH₃)CH₂CH₃, —C(O)—C(CH₃)₃, —C(O)—CH₂CH₂CH₂CH₂CH₃, —C(O)—CH₂CH(CH₃)CH₂CH₃, —C(O)—CH₂CH₂CH(CH₃)₂, —C(O)—CH₂CH₂CH₂CH₂CH₂CH₃, —C(O)—CH(CH₃)CH₂CH₂CH₂CH₃, —C(O)—CH₂CH(CH₃)CH₂CH₂CH₃, —C(O)—CH₂CH₂CH(CH₃)CH₂CH₃, —C(O)—CH₂CH₂CH₂CH(CH₃)₂, etc. Non-limiting examples of Y¹ when Y¹ is -alkylene-OH include —CH₂—OH, —CH₂CH₂—OH, —CH₂CH₂CH₂—OH, —CH(OH)CH₃, —CH₂CH(OH)CH₃, etc. Non-limiting examples of Y¹ when Y¹ is —C(O)-aryl include —C(O)-phenyl, —C(O)-naphthyl, etc. Non-limiting examples of Y¹ when Y¹ is —C(O)-haloalkyl include —C(O)—CF₃, —C(O)—CHF₂, —C(O)—CH₂F, —C(O)—CH₂CF₃, —C(O)—CF₂CF₃, —C(O)—CH₂Br, —C(O)—CH₂Cl, —C(O)—CCl₃, etc. Non-limiting examples of Y¹ when Y¹ is —C(O)O-alkyl include —C(O)—O—CH₃, —C(O)—O—CH₂CH₃, —C(O)—O—CH₂CH₂CH₃, —C(O)—O—CH(CH₃)₂, —C(O)—O—CH₂CH₂CH₂CH₃, —C(O)—O—CH₂CH(CH₃)₂, —C(O)—O—CH(CH₃)CH₂CH₃, —C(O)—O—C(CH₃)₃, —C(O)—O—CH₂CH₂CH₂CH₂CH₃, —C(O)—O—CH₂CH(CH₃)CH₂CH₃, —C(O)—O—CH₂CH₂CH(CH₃)₂, —C(O)—O—CH₂CH₂CH₂CH₂CH₂CH₃, —C(O)—O—CH(CH₃)CH₂CH₂CH₂CH₃, —C(O)—O—CH₂CH(CH₃)CH₂CH₂CH₃, —C(O)—O—CH₂CH₂CH(CH₃)CH₂CH₃, —C(O)—O—CH₂CH₂CH₂CH(CH₃)₂, etc. Non-limiting examples of Y¹ when Y¹ is —N(R²)C(O)-alkyl include —NH—C(O)-alkyl, —N(alkyl)-C(O)-alkyl, and —N(aryl)-C(O)-alkyl wherein the terms “alkyl” and “aryl” are as defined above. Non-limiting examples of Y¹ when Y¹ is —N(R²)C(O)—N(R²)₂ include —NHC(O)—NH₂, —NHC(O)—N(alkyl)₂, —NHC(O)—N(aryl)₂, —NHC(O)—NH-alkyl, —NHC(O)—NH-aryl, —N(alkyl)C(O)—NH-alkyl, —N(alkyl)C(O)—NH-aryl, —N(aryl)C(O)—NH-aryl, —N(aryl)C(O)—NH-aryl, etc. Non-limiting examples of Y¹ when Y¹ is —O-alkyl include —O—CH₃, —O—CH₂CH₃, —O—CH₂CH₂CH₃, —O—CH(CH₃)₂, —O—CH₂CH₂CH₂CH₃, —O—CH₂CH(CH₃)₂, —O—CH(CH₃)CH₂CH₃, —O—C(CH₃)₃, —O—CH₂CH₂CH₂CH₂CH₃, —O—CH₂CH(CH₃)CH₂CH₃, —O—CH₂CH₂CH(CH₃)₂, —O—CH₂CH₂CH₂CH₂CH₂CH₃, —O—CH(CH₃)CH₂CH₂CH₂CH₃, —O—CH₂CH(CH₃)CH₂CH₂CH₃, —O—CH₂CH₂CH(CH₃)CH₂CH₃, —O—CH₂CH₂CH₂CH(CH₃)₂, etc. Non-limiting examples of Y¹ when Y¹ is —O-haloalkyl include —O—CF₃, —O—CHF₂, —O—CH₂F, —O—CH₂CF₃, —O—CF₂CF₃, —O—CH₂Br, —O—CH₂Cl, —O—CCl₃, etc. Non-limiting examples of Y¹ when Y¹ is —O-alkylene-C(O)OH include —O—CH₂—C(O)OH, —O—CH₂CH₂—C(O)OH, —CH₂CH₂CH₂C(O)OH, —O—CH(CH₃)—C(O)OH, —O—CH(C(O)OH)CH₂CH(CH₃)₂, —O—CH(CH₃)CH₂CH₂—C(O)OH, etc. Non-limiting examples of Y¹ when Y¹ is —S-alkyl include —S—CH₃, —S—CH₂CH₃, —S—CH₂CH₂CH₃, —S—CH(CH₃)₂, —S—CH₂CH₂CH₂CH₃, —S—CH₂CH(CH₃)₂, —S—CH(CH₃)CH₂CH₃, —S—C(CH₃)₃, —S—CH₂CH₂CH₂CH₂CH₃, —S—CH₂CH(CH₃)CH₂CH₃, —S—CH₂CH₂CH(CH₃)₂, —S—CH₂CH₂CH₂CH₂CH₂CH₃, —S—CH(CH₃)CH₂CH₂CH₂CH₃, —S—CH₂CH(CH₃)CH₂CH₂CH₃, —S—CH₂CH₂CH(CH₃)CH₂CH₃, —S—CH₂CH₂CH₂CH(CH₃)₂, etc. Non-limiting examples of Y¹ when Y¹ is —S-haloalkyl include —S—CF₃, —S—CHF₂, —S—CH₂F, —S—CH₂CF₃, —S—CF₂CF₃, —S—CH₂Br, —S—CH₂Cl, —S—CCl₃, etc. Non-limiting examples of Y¹ when Y¹ is -alkylene-OH include —CH₂—OH, —CH₂CH₂—OH, —CH₂CH₂CH₂—OH, —CH(OH)CH₃, —CH₂CH(OH)CH₃, etc. Non-limiting examples of Y¹ when Y¹ is -alkylene-C(O)—O-alkyl include —O—CH₂—C(O)O—CH₃, —O—CH₂—C(O)O—CH₂CH₃, —O—CH₂CH₂—C(O)O—CH₂CH₃, —O—CH₂CH₂CH₂—C(O)O—CH₃, —O—CH₂CH₂—C(O)O—C(CH₃)₃, —O—CH(CH₃)—C(O)O—CH₃, —O—CH₂CH₂—C(O)O—CH₃, —O—CH(C(O)OCH₃)CH₂CH(CH₃)₂, —O—CH(CH₃)CH₂CH₂—C(O)O—CH₃, etc. Non-limiting examples of Y¹ when Y¹ is —O-alkylene-aryl include —O—CH₂-phenyl, —O—CH₂CH₂-phenyl, —O—CH(CH₃)-phenyl, —O—CH₂CH(CH₃)-phenyl, —OC(CH₃)₂-phenyl, —O—CH(CH₂CH₃)-phenyl, etc. Non-limiting examples of Y¹ when Y¹ is —N(R⁵)₂ include —NH₂, —N(CH₃)₂, —NH(CH₃), —NH(phenyl), —N(phenyl)₂, —NH—S(O)₂—CH₃, —NH—S(O)₂-cyclopropyl, —NH—C(O)—NH₂, —NH—C(O)—N(CH₃)₂, —NH—C(O)—CH₃, —NH—CH₂CH₂—OH, etc.

In some embodiments, the aryl or heteroaryl portions of any of the groups of Y¹ may be unsubstituted or substituted with one or more Z groups as defined herein.

In embodiments where Z is present, each Z is independently selected from the group consisting of alkyl, halo, haloalkyl, —OH, —O-alkyl, and —CN. The terms “alkyl”, “halo”, aloalkyl”, and “—O-alkyl” are as defined herein.

Also included within the scope of the invention are metabolites of compounds of Formula (I) or its various embodiments described herein, that is, compounds formed in vivo upon administration. Some examples of metablites include:

(i) where a compound of the invention contains a methyl group, an hydroxymethyl derivative thereof (e.g., —CH₃→OH or —C(R)₂H→—C(R)₂OH, wherein each R is, independently, any corresponding substituent in Formula (I));

(ii) where a compound of the invention contains an alkoxy group, an hydroxyl derivative thereof (—OR→—OH, wherein R is any corresponding substituent in Formula (I));

(iii) where a compound of the invention contains a tertiary amino agroup, a secondary amino derivative thereof (—N(R)₂→—NHR, wherein each R is, independently, any corresponding secondary or tertiary amino substitutent in Formula (I));

(iv) where a compound of the invention contains a secondary amino group, a primary derivative thereof (—NHR→—NH₂, wherein R is any corresponding secondary amino or primary amino substituent of Formula (I);

(v) where a compound of the invention contains a phenyl moiety, a phenol derivative thereof (-Ph→-PhOH);

(vi) where a compound of the invention contains an amide group, a carboxylic acid derivative thereof (—CONH₂→—COOH).

As used throughout the specification, the following terms, unless otherwise indicated, shall be understood to have the following meanings:

The term “Patient” includes humans and/or other animals. Animals include mammals and non-mammalian animals. Mammals include humans and other mammalian animals. In some embodiments, the patient is a human. In other embodiments, the patient is non-human. In some embodiments, non-human animals include companion animals. Examples of companion animals include house cats (feline), dogs (canine), rabbits, horses (equine), guinea pigs, rodents (e.g., rats, mice, gerbils, or hamsters), primates (e.g., monkeys), and avians (e.g., pigeons, doves, parrots, parakeets, macaws, or canaries). In some embodiments, the animals are felines (e.g., house cats). In some embodiments, the animals are canines. Canines include, for example, wild and zoo canines, such as wolves, coyotes, and foxes. Canines also include dogs, particularly domestic dogs, such as, for example, pure-bred and/or mongrel companion dogs, show dogs, working dogs, herding dogs, hunting dogs, guard dogs, police dogs, racing dogs, and/or laboratory dogs. In some embodiments, non-human animals include wild animals; livestock animals (e.g., animals raised for food and/or other products, such as, for example, meat, poultry, fish, milk, butter, eggs, fur, leather, feathers, and/or wool); beasts of burden; research animals; companion animals; and animals raised for/in zoos, wild habitats, and/or circuses. In other embodiments, non-human animals include primates, such as monkeys and great apes. In other embodiments, animals include bovine (e.g., cattle or dairy cows), porcine (e.g., hogs or pigs), ovine (e.g., goats or sheep), equine (e.g., horses), canine (e.g., dogs), feline (e.g., house cats), camels, deer, antelope, rabbits, guinea pigs, rodents (e.g., squirrels, rats, mice, gerbils, or hamsters), cetaceans (e.g., whales, dolphins, or porpoises), pinnipeds (e.g., seals or walruses). In other embodiments, animals include avians. Avians include birds associated with either commercial or noncommercial aviculture. These include, for example, Anatidae, such as swans, geese, and ducks; Columbidae, such as doves and pigeons (e.g., such as domestic pigeons); Phasianidae, such as partridges, grouse and turkeys; Thesienidae, such as domestic chickens; Psittacines, such as parakeets, macaws, and parrots (e.g., parakeets, macaws, and parrots raised for pets or collector markets; game birds; and ratites, such as ostriches. In other embodiments, animals include fish. Fish include, for example, the Teleosti grouping of fish (i.e., teleosts), such as, for example, the Salmoniformes order (which includes the Salmonidae family) and the Perciformes order (which includes the Centrarchidae family). Examples of fish include the Salmonidae family, the Serranidae family, the Sparidae family, the Cichlidae family, the Centrarchidae family, the three-Line Grunt (Parapristipoma trilineatum), and the Blue-Eyed Plecostomus (Plecostomus spp). Additional examples of fish include, for example, catfish, sea bass, tuna, halibut, arctic charr, sturgeon, turbot, flounder, sole, carp, tilapia, striped bass, eel, sea bream, yellowtail, amberjack, grouper, and milkfish. In other embodiments, animals include marsupials (e.g., kangaroos), reptiles (e.g., farmed turtles), amphibians (e.g., farmed frogs), crustaceans (e.g., lobsters, crabs, shrimp, or prawns), mollusks (e.g., octopus and shellfish), and other economically-important animals.

“Body Condition Score” refers to an assessment of an animal's weight for age and weight for height ratios, and its relative proportions of muscle and fat. The assessment is made by eye, on the basis of amount of tissue cover between the various points of reference. The grading may be expressed as a score ranging from 1 to 8. As used herein, Body Condition Scores of 1 to 8 are described as follows:

| Score | Description |
|---|---|
| 1 | Emaciated. Ribs, lumbar vertebrae, pelvic bones |
| | and all bony prominences evident from a distance. |
| | No discernable body fat. Obvious loss of muscle |
| | mass. |
| 2 | Very thin. Ribs, lumbar vertebrae and pelvic bond |
| | easily visible. No palpable fat. Some evidence of |
| | other bony prominence. Minimal loss of muscle |
| | mass. |
| 3 | Thin. Ribs easily palpated and may be visible with |
| | no palpable fat. Tops of lumbar vertebrae visible. |
| | Pelvic bones becoming prominent. Obvious waist |
| | and lack of abdominal tuck. |
| 4 | Underweight. Ribs easily palpable with minimal fat |
| | covering. Waist easily noted from above. Abdominal |
| | tuck evident. |
| 5 | Ideal. Ribs palpable without excess fat covering. |
| | Waist observed behind ribs when viewed from above. |
| | Abdomen tucked when viewed from the side. |
| 6 | Overweight. Ribs palpable with slight excess fat |
| | covering. Waist is discernable viewed from above, |
| | but is not prominent. Abdominal tuck apparent. |
| 7 | Heavy. Ribs palpable with difficulty, heavy fat cover. |
| | Noticeable fat deposits over lumbar area and base of |
| | tail. Waist absent or barely visible. Abdominal tuck |
| | may be present. |
| 8 | Obese. Ribs not palpable under very heavy fat |
| | cover, or palpable only with significant pressure. |
| | Heavy fat deposits over lumbar area and base of tail. |
| | Waist absent. NO abdominal tuck. Obvious |
| | abdominal distension may be present. |

“Alkyl” means an aliphatic hydrocarbon group which may be straight or branched and comprising about 1 to about 20 carbon atoms in the chain. In one embodiment alkyl groups contain about 1 to about 12 carbon atoms in the chain. In another embodiment alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. Non-limiting examples of suitable alkyl groups include methyl, ethyl, n-propyl, isopropyl, n-butyl, t-butyl, n-pentyl, heptyl, nonyl, or decyl.

“Alkylene” means a divalent group obtained by removal of a hydrogen atom from an alkyl group that is defined above. Non-limiting examples of alkylene include methylene, ethylene and propylene. In one embodiment, alkylene groups have about 1-18 carbon atoms in the chain, which may be straight or branched. In another embodiment, alkylene groups have about 1-12 carbon atoms in the chain, which may be straight or branched. In another embodiment, alkylene groups may be lower alkylenes. “Lower alkylene” means an alkylene having about 1 to 6 carbon atoms in the chain, which may be straight or branched.

“Alkenyl” means an aliphatic hydrocarbon group containing at least one carbon-carbon double bond and which may be straight or branched and comprising about 2 to about 15 carbon atoms in the chain. In one embodiment alkenyl groups have about 2 to about 12 carbon atoms in the chain. In another embodiment alkenyl groups have about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The term “substituted alkenyl” means that the alkenyl group may be substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkenylene” means a divalent group obtained by removal of a hydrogen atom from an alkenyl group that is defined above.

“Alkynyl” means an aliphatic hydrocarbon group containing at least one carbon-carbon triple bond and which may be straight or branched and comprising about 2 to about 15 carbon atoms in the chain. In one embodiment alkynyl groups have about 2 to about 12 carbon atoms in the chain. In another embodiment alkynyl groups have about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl. The term “substituted alkynyl” means that the alkynyl group may be substituted by one or more substituents which may be the same or different, each substituent being independently selected from the group consisting of alkyl, aryl and cycloalkyl.

“Aryl” (sometimes abbreviated “ar” or “Ar”) means an aromatic monocyclic or multicyclic ring system comprising about 6 to about 14 carbon atoms, or about 6 to about 10 carbon atoms. The aryl group can be optionally substituted with one or more “ring system substituents” which may be the same or different, and are as defined herein. Non-limiting examples of suitable aryl groups include phenyl, naphthyl, and biphenyl.

“Aryloxy” means a —O-aryl group, wherein aryl is defined as above. the aryloxy group is attached to the parent moiety through the ether oxygen.

“Arylene” means a divalent aryl group obtained by the removal of a hydrogen atom from an aryl group as defined above. Non-limiting examples of arylenes include, for example, 1,2-phenylene, 1,3-phenylene, or 1,4-phenylene.

“Heteroaryl” means an aromatic monocyclic or multicyclic ring system comprising about 5 to about 14 ring atoms, or about 5 to about 10 ring atoms, in which one or more of the ring atoms is an element other than carbon, for example nitrogen, oxygen or sulfur, alone or in combination. In one embodiment heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like.

“Cycloalkyl” means a non-aromatic mono- or multicyclic ring system comprising about 3 to about 13 carbon atoms, or about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalin, norbornyl, adamantyl and the like.

“Cycloalkylene” means a divalent cycloalkyl group obtained by the removal of a hydrogen atom from a cycloalkyl group as defined above. Non-limiting examples of cycloalkylenes include:

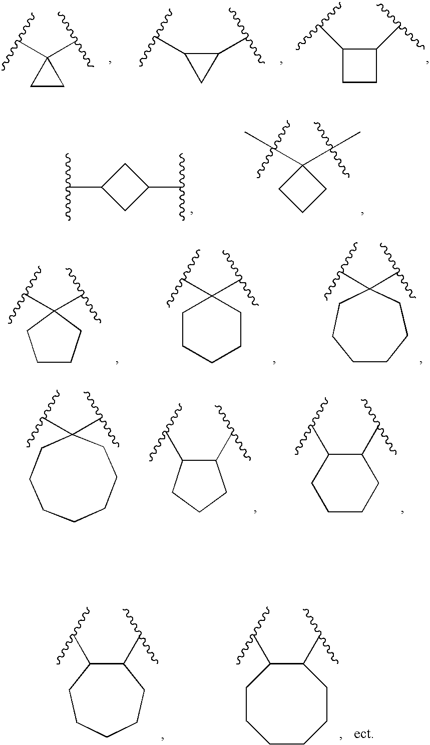

“Alkylene containing one or more cycloalkylene groups” means an alkylene group is bound to one or both of the open valancies of a cycloalkylene group. Similarly, “alkenylene (or alkynylene) containing one or more cycloalkylene groups” means an alkenylene (or alkynylene) group bound to one or both of the open valancies of a cycloalkylene group.

“Heterocycloalkyl” means a non-aromatic saturated monocyclic or multicyclic ring system comprising about 3 to about 10 ring atoms, or about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. In one embodiment heterocycloalkyls contain about 5 to about 6 ring atoms. The prefix aza, oxa or thia before the heterocycloalkyl root name means that at least a nitrogen, oxygen or sulfur atom respectively is present as a ring atom. The heterocycloalkyl can be optionally substituted by one or more “ring system substituents” which may be the same or different, and are as defined herein. The nitrogen or sulfur atom of the heterocycloalkyl can be optionally oxidized to the corresponding N-oxide, S-oxide or S,S-dioxide. Non-limiting examples of suitable monocyclic heterocycloalkyl rings include piperidyl, pyrrolidinyl, piperazinyl, morpholinyl, thiomorpholinyl, thiazolidinyl, 1,3-dioxolanyl, 1,4-dioxanyl, tetrahydro-pyranyl, tetrahydrofuranyl, tetrahydrothiophenyl, tetrahydrothiopyranyl, and the like.

“Heterocycloalkenyl” means a non-aromatic unsaturated monocyclic or multicyclic ring system comprising about 3 to about 10 ring atoms, or about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Heterocycloalkenyls have at least one double bond, wherein said double bond may be between two ring carbon atoms, between a ring carbon atom and a ring heteroatom (e.g., between a ring carbon atom and a ring nitrogen atom), or between two ring heteroatoms (e.g., between two ring nitrogen atoms). If more than one double bond is present in the ring, each double bond is independently defined as described herein. In another embodiment heterocycloalkenyls contain about 5 to about 6 ring atoms. The prefix aza, oxa or thia before the heterocycloalkenyl root name means that at least a nitrogen, oxygen or sulfur atom respectively is present as a ring atom. The heterocycloalkenyl can be optionally substituted by one or more “ring system substituents” which may be the same or different, and are as defined herein. The nitrogen or sulfur atom of the heterocycloalkenyl can be optionally oxidized to the corresponding N-oxide, S-oxide or S,S-dioxide. Non-limiting examples of suitable monocyclic heterocycloalkenyl rings include thiazolinyl, 2,3-dihydro-1H-pyrrolyl, 2,5-dihydro-1H-pyrrolyl, 3,4-dihydro-2H-pyrrolyl, 2,3-dihydro-furan, 2,5-dihydro-furan, etc.

“Benzo-fused heterocycloalkenyl” means a heterocycloalkenyl, as defined above, to which one or more phenyl rings has been fused, so that each phenyl ring shares two ring carbon atoms with the cycloalkyl ring. In one embodiment, the benzo-fused heterocycloalkenyl group is attached to the rest of the molecule through the heterocycloalkenyl group. In another embodiment, the benzo-fused heterocycloalkenyl group is attached to the rest of the molecule through the benzyl group. Non-limiting examples of benzo-fused cycloalkyls are 4H-chromene, chromene-4-one, 1H-isochromene, etc.

“Benzo-fused cycloalkyl” means a cycloalkyl, as defined above, to which one or more phenyl rings has been fused, so that each phenyl ring shares two ring carbon atoms with the cycloalkyl ring. In one embodiment, the benzo-fused cycloalkenyl group is attached to the rest of the molecule through the cycloalkenyl group. In another embodiment, the benzo-fused cycloalkenyl group is attached to the rest of the molecule through the benzyl group. Non-limiting examples of benzo-fused cycloalkyls are indanyl and tetradehydronaphthyl:

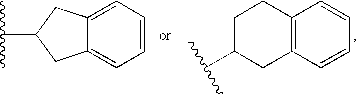

and non-limiting examples of a dibenzo-fused cycloalkyls are fluorenyl:

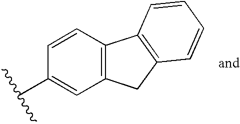

acenaphthenyl:

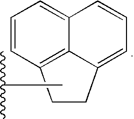

“Benzo-fused heterocycloalkyl” means a heterocycloalkyl, as defined above, to which one or more phenyl rings has been fused, so that each phenyl ring shares two ring carbon atoms with the heterocycloalkyl ring. In one embodiment, the benzo-fused heterocycloalkyl group is attached to the rest of the molecule through the heterocycloalkenyl group. In another embodiment, the benzo-fused heterocycloalkyl group is attached to the rest of the molecule through the benzyl group. A non-limiting example of a benzo-fused heterocycloalkyls is 2,3-dihydro-benzo[1,4]dioxinyl.

“Cycloalkenyl” means a non-aromatic mono or multicyclic ring system comprising about 3 to about 10 carbon atoms, or about 5 to about 10 carbon atoms, which contains at least one carbon-carbon double bond. In one embodiment cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cycloheptenyl, and the like. Non-limiting example of a suitable multicyclic cycloalkenyl is norbornylenyl.

“Halo” (or “halogeno” or “halogen”) means fluoro, chloro, bromo, or iodo groups. Preferred are fluoro, chloro or bromo, and more preferred are fluoro and chloro.

“Haloalkyl” means an alkyl as defined above wherein one or more hydrogen atoms on the alkyl are replaced by a halo group as defined above.

“Ring system substituent” means a substituent attached to an aromatic or non-aromatic ring system which, for example, replaces an available hydrogen on the ring system. Ring system substituents may be the same or different, and are defined as described herein.

“Alkoxy” means an —O-alkyl group in which the alkyl group is as previously described. Non-limiting examples of suitable alkoxy groups include methoxy, ethoxy, n-propoxy, isopropoxy, n-butoxy and heptoxy. The bond to the parent moiety is through the ether oxygen.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

When used herein, the term “independently”, in reference to the substitution of a parent moiety with one or more substituents, means that the parent moiety may be substituted with any of the listed substituents, either individually or in combination, and any number of chemically possible substituents may be used. As a non-limiting example, a phenyl independently substituted with one or more alkyl or halo substituents can include, chlorophenyl, dichlorophenyl, trichlorophenyl, tolyl, xylyl, 2-chloro-3-methylphenyl, 2,3-dichloro-4-methylphenyl, etc.

As used herein, the term “composition” is intended to encompass a product comprising the specified ingredients in the specified amounts, as well as any product which results, directly or indirectly, from combination of the specified ingredients in the specified amounts.

The wavy line [symbol] as a bond generally indicates a mixture of, or either of, the possible isomers, e.g., containing (R)- and (S)-stereochemistry. For example,

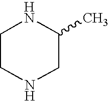

means containing both

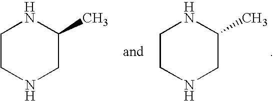

Moreover, when the stereochemistry of a chiral center (or stereogenic center) is not expressly indicated, a mixture of, or any of the individual possible isomers are contemplated. Thus, for example,

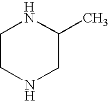

means containing

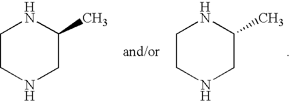

Lines drawn into the ring systems, such as, for example:

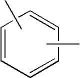

indicate that the indicated line (bond) may be attached to any of the substitutable ring carbon atoms. Hetero-atom containing ring systems, when present in a compound according to the invention, can be optionally substituted with a ring system substitutent at an available ring carbon atom, an available ring heteroatom, or both, where allowed by appropriate valency rules.

As well known in the art, a bond drawn from a particular atom wherein no moiety is depicted at the terminal end of the bond indicates a methyl group bound through that bond to the atom, unless stated otherwise. For example:

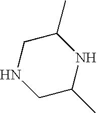

represents

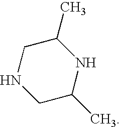

It should also be noted that any carbon or heteroatom with unsatisfied valences in the text, schemes, examples, structural formulae, and any Tables herein is assumed to have the hydrogen atom or atoms to satisfy the valences.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in any Formula (e.g., Formula I), its definition on each occurrence is independent of its definition at every other occurrence.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the present invention may also exist as, or optionally be converted to a solvate. The preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The compounds of Formula (I) form salts that are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a piperazine, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Intl Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The compounds of the present invention may contain asymmetric or chiral centers, and, therefore, exist in different stereoisomeric forms. It is intended that all stereoisomeric forms of the compounds of the invention as well as mixtures thereof, including racemic mixtures, form part of the present invention. In addition, the present invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, those of ordinary skill in the art will recognize any compounds of the present invention that may be atropisomers (e.g., substituted biaryls). Such atropisomers are considered as part of this invention. Enantiomers can also be separated by use of chiral HPLC column.

Compounds of Formula (I), and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula I (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula (I), and of the salts, solvates and prodrugs of the compounds of Formula (I), are intended to be included in the present invention.

In still another embodiment, the present invention provides a composition comprising at least one compound of Formula (I), or a pharmaceutically acceptable salt, solvate, isomer, or ester thereof, and a pharmaceutically acceptable carrier.

The term “pharmaceutical composition” is also intended to encompass both the bulk composition and individual dosage units comprised of more than one (e.g., two, three, four, or more) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

Unit dosage forms, without limitation, can include tablets, pills, capsules, sustained release pills, sustained release tablets, sustained release capsules, powders, granules, or in the form of solutions or mixtures (i.e., elixirs, tinctures, syrups, emulsions, suspensions). For example, one or more compounds of Formula (I), or salts or solvates thereof, may be combined, without limitation, with one or more pharmaceutically acceptable liquid carriers such as ethanol, glycerol, or water, and/or one or more solid binders such as, for example, starch, gelatin, natural sugars (e.g., glucose or β-lactose), and/or natural or synthetic gums (e.g., acacia, tragacanth, or sodium alginate), carboxymethylcellulose, polyethylene glycol, waxes and the like, and/or disintegrants, buffers, preservatives, anti-oxidants, lubricants, flavorings, thickeners, coloring agents, emulsifiers and the like. In addition, the unit dosage forms can include, without limitation, pharmaceutically acceptable lubricants (e.g., sodium oleate, sodium stearate, magnesium stearate, sodium benzoate, sodium acetate, and sodium chloride) and disintegrators (e.g., starch, methyl cellulose, agar, bentonite, and xanthan gum). The amount of excipient or additive can range from about 0.1 to about 90% by weight of the total weight of the treatment composition. One skilled in the art understands that the amount of carrier(s), excipients, and additives (if present) can vary.

In another embodiment, the present invention provides a method of treating, reducing, or ameliorating hepatic lipidosis and/or fatty liver disease (including but not limited to non-alcoholic fatty liver disease) in a patient in need thereof, comprising administering to said patient an effective amount of a composition comprising at least one compound of Formula (I), or a pharmaceutically acceptable salt, solvate, or ester thereof and a pharmaceutically acceptable carrier.

In another embodiment, the present invention provides a method of reducing body condition score (BCS) in a patient in need thereof, comprising administering to said patient an effective amount of a composition comprising at least one compound of Formula (I), or a pharmaceutically acceptable salt, solvate, or ester thereof (optionally together with at least one additional active agent) and one or more pharmaceutically acceptable carriers. In one embodiment, the patient is a non-human animal. In one embodiment, the patient is a companion animal. In one embodiment, BCS is reduced from obese to ideal. In another embodiment, BCS is reduced from obese to heavy, overweight, or ideal. In another embodiment, BCS is reduced from obese to heavy. In another embodiment, BCS is reduced from obese to overweight. In another embodiment, BCS is reduced from heavy to overweight or ideal. In another embodiment, BCS is reduced from heavy to ideal. In another embodiment, BCS is reduced from overweight to ideal.

In other embodiments, the present invention provides a method of reducing the abdominal girth in a patient in need thereof. The method comprises administering an effective amount of a composition comprising at least one compound of Formula (I), or a pharmaceutically acceptable salt, solvate, or ester thereof (optionally together with at least one additional active agent) and one or more pharmaceutically acceptable carriers. In some embodiments, the patient is a non-human animal. In some such embodiments, for example, the patient may be a companion mammal, such as a dog, cat, or horse. Girth measurements are taken at the widest point behind the last rib and in front of the pelvis.

In other embodiments, the present invention provides a method of repartitioning, wherein energy of an animal is partitioned away from fat deposition toward protein accretion. The method comprising administering to said patient an effective amount of a composition comprising at least one compound of Formula (I), or a pharmaceutically acceptable salt, solvate, or ester thereof (optionally together with at least one additional active agent) and one or more pharmaceutically acceptable carriers. In some embodiments, the patient is a non-human animal. In some such embodiments, for example, the patient may be a food animal, such as a bovine animal, swine animal, sheep, goat, or poultry animal (chicken, turkey, etc.). In other embodiments, the animal is an equine animal.

In other embodiments, the present invention provides a method of treating, reducing, or ameliorating a disease or condition selected from the group consisting of metabolic syndrome, obesity, waist circumference, abdominal girth, lipid profile, insulin sensitivity, neuroinflammatory disorders, cognitive disorders, psychosis, addictive behavior, gastrointestinal disorders, and cardiovascular conditions, in a patient in need thereof, comprising administering to said patient an effective amount of at least one compound of Formula (I), or a pharmaceutically acceptable salt, solvate, isomer, or ester thereof.

In another embodiment, the present invention provides a method of treating, reducing, or ameliorating a disease or condition selected from psychic disorders, anxiety, schizophrenia, depression, abuse of psychotropes, abuse and/or dependence of a substance, alcohol dependency, nicotine dependency, neuropathies, migraine, stress, epilepsy, dyskinesias, Parkinson's disease, amnesia, senile dementia, Alzheimer's disease, eating disorders, diabetes type II or non insulin dependent diabetes (NIDD), gastrointestinal diseases, vomiting, diarrhea, urinary disorders, infertility disorders, inflammations, infections, cancer, neuroinflammation, in particular in atherosclerosis, or the Guillain-Barr syndrome, viral encephalitis, cerebral vascular incidents and cranial trauma.

In yet another embodiment, the present invention provides a method of treating, reducing, or ameliorating obesity, in a patient in need thereof, comprising administering to said patient an effective amount of at least one compound of Formula (I), or a pharmaceutically acceptable salt, solvate, isomer, or ester thereof.

In yet other embodiments, the present invention provides a method of treating, reducing, or ameliorating metabolic syndrome, obesity, waist circumference, abdominal girth, lipid profile, insulin sensitivity, neuroinflammatory disorders, cognitive disorders, psychosis, addictive behavior, gastrointestinal disorders, and cardiovascular conditions, in a patient in need thereof, comprising administering to said patient an effective amount of a composition comprising at least one compound of Formula (I), or a pharmaceutically acceptable salt, solvate, isomer, or ester thereof and a pharmaceutically acceptable carrier.

In yet another embodiment, the present invention provides a method of treating, reducing, or ameliorating obesity, in a patient in need thereof, comprising administering to said patient an effective amount of a composition comprising at least one compound of Formula (I), or a pharmaceutically acceptable salt, solvate, isomer, or ester thereof and a pharmaceutically acceptable carrier.

The compounds of Formula (I) can be useful as CB₁ receptor antagonists for treating, reducing, or ameliorating metabolic syndrome, obesity, waist circumference, abdominal girth, lipid profile, insulin sensitivity, neuroinflammatory disorders, cognitive disorders, psychosis, addictive behavior (e.g., smoking cessation), gastrointestinal disorders, and cardiovascular conditions (e.g., elevated cholesterol and triglyceride levels). It is contemplated that the compounds of Formula (I) of the present invention, or pharmaceutically acceptable salts, solvates, or esters thereof, can be useful in treating one or more the conditions or diseases listed above. In particular, the compounds of Formula (I) of the present invention are useful in treating obesity.

“Effective amount” or “therapeutically effective amount” is meant to describe an amount of compound or a composition of the present invention effective in antagonizing a CB₁ receptor and thus producing the desired therapeutic effect in a suitable patient.

The selective CB₁ receptor antagonist compound of Formula (I), or a pharmaceutically acceptable salt, solvate, isomer, or ester thereof, can be administered in a therapeutically effective amount and manner to treat the specified condition. The daily dose of the selective CB₁ receptor antagonist of Formula (I) (or pharmaceutically acceptable salts, solvates, or esters thereof) administered to a mammalian patient or subject can range from about 1 mg/kg to about 50 mg/kg (where the units mg/kg refer to the amount of selective CB₁ receptor antagonist compound of Formula (I) per kg body weight of the patient), or about 1 mg/kg to about 25 mg/kg, or about 1 mg/kg to about 10 mg/kg.

Alternatively, the daily dose can range from about 1 mg to about 50 mg, or about 1 mg to about 25 mg, or about 5 mg to about 20 mg. In one embodiment, the daily dose can range from about 0.01 mg/kg to about 1 mg/kg. In another embodiment, the daily dose can range from about 1 mg/kg to about 10 mg/kg. In another embodiment, the daily dose can range from about 1 mg/kg to about 25 mg/kg. Although a single administration of the selective CB₁ receptor antagonist compound of Formula (I), or salts, solvates, or esters thereof, can be efficacious, multiple dosages can also be administered. The exact dose, however, can readily be determined by the attending clinician and will depend on such factors as the potency of the compound administered, the age, weight, condition and response of the patient.

The treatment compositions of the present invention can be administered in any conventional dosage form, preferably an oral dosage form such as a capsule, tablet, powder, cachet, suspension, or solution. The formulations and pharmaceutical compositions can be prepared using conventional pharmaceutically acceptable and conventional techniques.

In the veterinary context, in particular, the compounds of this invention can be administered to an animal patient in one or more of a variety of routes. For example, the compounds may be administered orally via, for example, a capsule, bolus, tablet (e.g., a chewable treat), powder, drench, elixir, cachet, solution, paste, suspension, or drink (e.g., in the drinking water or as a buccal or sublingual formulation). The compounds may alternatively (or additionally) be administered via a medicated feed (e.g., when administered to a non-human animal) by, for example, being dispersed in the feed or used as a top dressing or in the form of pellets or liquid which is added to the finished feed or fed separately. The compounds also may be administered (alternatively or additionally) parenterally via, for example, an implant or an intraruminal, intramuscular, intravascular, intratracheal, or subcutaneous injection. It is contemplated that other administration routes (e.g., topical, intranasal, rectal, etc.) may be used as well. Formulations for any such administration routes can be prepared using, for example, various conventional techniques known in the art. In some embodiments, from about 5 to about 70% by weight of the veterinary formulation (e.g., a powder or tablet) comprises active ingredient.

Suitable solid carriers are known in the art, and include, for example, magnesium carbonate, magnesium stearate, talc, sugar, and lactose. Tablets, powders, cachets, and capsules can be used as solid dosage forms suitable for oral administration.

To prepare suppositories, the active ingredient may be dispersed homogeneously into a melted wax that melts at low temperatures (e.g., a mixture of fatty acid glycerides or cocoa butter). Such dispersion may be achieved by, for example, stirring. The molten homogeneous mixture may be poured into convenient-sized molds, allowed to cool, and, thereby, solidify.

Liquid form preparations include solutions, suspensions, and emulsions. In some embodiments, for example, water or water-propylene glycol solutions are used for parenteral injection. Liquid form preparations also may include solutions for intranasal administration.

Aerosol preparations suitable for inhalation may include solutions and solids in powder form, which may be combined with a pharmaceutically acceptable carrier, such as an inert compressed gas.

Solid form preparations also include, for example, preparations that are intended to be converted, shortly before use, to liquid form preparations for either oral or parenteral administration. Such liquid forms include solutions, suspensions, and emulsions.

In some embodiments, the compounds of this invention are formulated for transdermal delivery. Transdermal compositions may be, for example, creams, lotions, aerosols, and/or emulsions, and can be included in a transdermal patch of the matrix or reservoir type as are conventional in the art for this purpose.

It is contemplated that the active can be incorporated into animal feed. A suitable amount of compound of the present invention can be placed into a commercially available feed product to achieve desired dosing levels. The amount of compound of the present invention incorporated into the feed will depend on the rate at which the animals are fed. Compounds or compositions of the present invention can be incorporated into feed mixtures before pelleting. Alternatively, the medicated feed is formed by coating feed pellets with a compound(s) or compositions of the present invention.

In some embodiments, the present invention provides a method of treating fish for an indication described herein. Such methods include administering an effective amount of an inventive compound (or compounds) of the invention (optionally together with one or more additional active agents as described herein) to a fish or a fish population. Administration generally is achieved by either feeding the fish an effective amount of the inventive compound or by immersing the fish in a solution that contains an effective amount of the inventive compound. It is to be further understood that the inventive compound can be administered by application of the inventive compound(s) to a pool or other water-holding area containing the animal, and allowing the fish to absorb the compound through its gills, or otherwise allowing the dosage of the inventive compound to be taken in. For individual treatment of specific animals, such as a particular fish (e.g., in a veterinary or aquarium setting), direct injection or injection of osmotic release devices comprising the inventive compound, alone or in combination with other agents, is an optional method of administering the inventive compound. Suitable routes of administration include, for example, intravenous, subcutaneous, intramuscular, spraying, dipping, or adding the compound directly into the water in a holding volume.

In other embodiments, the present invention provides a composition comprising: (a) at least one compound of Formula (I), or a pharmaceutically acceptable salt, solvate, isomer or ester thereof, and (b) at least one additional active ingredient. Thus, it is contemplated that any of the indications suitable for treatment by at least one compound of Formula (I) may be treated using at least one compound of Formula (I) together with at least one additional active ingredient. Such additional active ingredient(s) may be combined with one or more compounds of the invention to form a single composition for use or the active ingredients may be formulated for separate (simultaneous or sequential) administration. Such additional active ingredients are described herein or are know to those of ordinary skill in the art. Non-limiting examples include centrally acting agents and peripherally acting agents. Non-limiting examples of centrally acting agents include histamine-3 receptor antagonists such as those disclosed in U.S. Pat. No. 6,720,328 (incorporated herein by reference). Non-limiting examples of such histamine H-3 receptor antagonists include the compound having a structure (as well as salts, solvates, isomers, esters, prodrugs, etc. thereof):

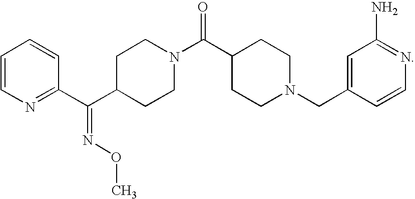

Other non-limiting examples of histamine-3 receptor antagonists include those disclosed in U.S. Pat. No. 7,105,505 (incorporated herein by reference). Non-limiting examples of such histamine H-3 receptor antagonists include the compound having a structure (as well as salts, solvates, isomers, esters, prodrugs, etc. thereof):

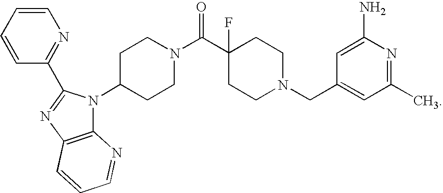

Additional non-limiting examples of centrally acting agents include neuropeptide Y5 (NPY5) antagonists such as those disclosed in U.S. Pat. No. 6,982,267 (incorporated herein by reference). Non-limiting examples of such histamine NPY5 receptor antagonists include the compound having a structure (and salts, solvates, isomers, esters, prodrugs, etc. thereof):

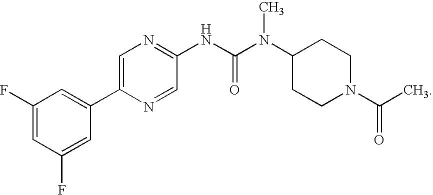

Non-limiting examples of peripherally acting agents include microsomal triglyceride transfer protein (MTP) inhibitors. Non-limiting examples of MTP inhibitors include dirlotapide (Slentrol™, Pfizer). Additional non-limiting examples of additional active ingredients are described herein.

In another embodiment, the present invention provides a composition comprising: (a) at least one compound of Formula (I), or a pharmaceutically acceptable salt, solvate, isomer or ester thereof, and (b) at least one cholesterol lowering compound.

Therapeutic combinations also are provided comprising: (a) a first amount of at least one selective CB₁ receptor antagonist, or a pharmaceutically acceptable salt, solvate, isomer or ester, thereof; and (b) a second amount of at least one cholesterol lowering compound, wherein the first amount and the second amount together comprise a therapeutically effective amount for the treatment or prevention of a vascular condition, diabetes, obesity, hyperlipidemia, metabolic syndrome, or lowering a concentration of a sterol in the plasma of a subject.

Pharmaceutical compositions for the treatment or prevention of a vascular condition, diabetes, obesity, hyperlipidemia, metabolic syndrome, or lowering a concentration of a sterol in the plasma of a subject comprising a therapeutically effective amount of the above compositions or therapeutic combinations and a pharmaceutically acceptable carrier also are provided.

In still yet another embodiment, the compositions and combinations of the present invention comprise at least one compound of Formula (I), or a pharmaceutically acceptable salt, solvate, isomer, or ester thereof, and one or more anti-diabetic drugs. Non-limiting examples of anti-diabetic drugs include sulffonyl ureas, meglitinides, biguanides, thiazolidinediones, alpha glucosidase inhibitors, incretin mietics, DPP-IV (dipeptidyl peptidase-4 or DPP-4) inhibitors, amylin analogues, insulin (including insulin by mouth), and herbal extracts.

Non-limiting examples of sulfonylureas include tolbutamide (Orinase®), acetohexamide (Dymelor®), tolazamide (Tolinase®), chlorpropamide (Diabinese®), glipizide (Glucotrol(RO), glyburide (Diabeta®, Micronase®, and Glynase®), glimepiride (Amaryl®), and gliclazide (Diamicron®).

Non-limiting examples of meglitinides include repaglinide (Prandin®), and mateglinide (Starlix®).

Non-limiting examples of biguanides include metformin (Glucophage®).

Non-limiting examples of thaizolidinediones, also known as glitazines, include rosiglitazone (Avandia®), pioglitazone (Actos®), and troglitazine (Rezulin®).

Non-limiting examples of gludosidase inhibitors include miglitol (Glyset®) and acarbose (Precose/Glucobay®).

Non-limiting examples of incretin mimetics include GLP agonists such as exenatide and exendin-4, marketed as Byetta® (Amylin Pharmaceuticals, Inc. and Eli Lilly and Company.)

Non-limiting examples of Amylin analogues include pramlintide acetate (Symlin® Amylin Pharmaceuticals, Inc.).

Non-limiting examples of DPP4 inhibitors and other anti-diabetic drugs include the following: sitagliptin (marketed as Januvia®, available from Merck, pyrazine-based DPP-IV derivatives such as those disclosed in WO-2004085661, bicyclictetrahydropyrazine DPP IV inhibitors such as those disclosed in WO-03004498, PHX1149 (available from Phenomix, Inc.), ABT-279 and ABT-341 (available from Abbott, see WO-2005023762 and WO-2004026822), ALS-2-0426 (available Alantos and Servier), AR12243 (available from Arisaph Pharmaceuticals Inc., U.S. Pat. No. 06,803,357 and U.S. Pat. No. 06,890,898), boronic acid DPP-IV inhibitors such as those described in US patent application Ser. No. 06/303,661, BI-A and BI-B (available from Boehringer Ingelheim), xanthine-based DPP-IV inhibitors such as those described in WO-2004046148, WO-2004041820, WO-2004018469, WO-2004018468 and WO-2004018467, saxagliptin (Bristol-Meyers Squibb and Astra Zenica), Biovitrim (developed by Santhera Pharmaceuticals (formerly Graffinity)), MP-513 (Mitsubishi Pharma), NVP-DPP-728 (qv) and structurally related 1-((S)-gamma-substituted prolyl)-(S)-2-cyanopyrrolidine compounds and analogs of NVP-DPP-728 (qv), DP-893 (Pfizer), vildagliptin (Novartis Institutes for BioMedical Research Inc), tetrahydroisoquinoline 3-carboxamide derivatives such as those disclosed in U.S. patent application Ser. No. 06/172,081, N-substituted 2-cyanopyrrolidines, including LAF-237, such as those disclosed in PCT Publication Nos. WO-00034241, WO-00152825, WO-02072146 and WO-03080070, WO-09920614, WO-00152825 and WO-02072146, SYR-322 (Takeda), denagliptin, SNT-189546, RO-0730699, BMS-2, Aurigene, ABT-341, Dong-A, GSK-2, HanAll, LC-15-0044, SYR-619, Bexel, alogliptin benzoate, and ALS-2-0426. Non-limiting examples of other anti-diabetic drugs include metformin, thiazolidinediones (TZD), and sodium glucose cotransporter-2 inhibitors such as dapagliflozin (Bristol Meyers Squibb) and sergliflozin (GlaxoSmithKline), and FBPase (fructose 1,6-bisphosphatase) inhibitors.

In still yet another embodiment, the compositions and combinations of the present invention comprise at least one compound of Formula (I), or a pharmaceutically acceptable salt, solvate, isomer or ester thereof, and at least one sterol absorption inhibitor or at least one 5α-stanol absorption inhibitor.

In still yet another embodiment of the present invention, there is provided a therapeutic combination comprising: (a) a first amount of at least one compound of Formula (I), or a pharmaceutically acceptable salt, solvate, isomer or ester thereof; and (b) a second amount of at least one cholesterol lowering compound; wherein the first amount and the second amount together comprise a therapeutically effective amount for the treatment or prevention of one or more of a vascular condition, diabetes, obesity, metabolic syndrome, or lowering a concentration of a sterol in the plasma of a subject.

In still yet another embodiment, the present invention provides for a pharmaceutical composition for the treatment or prevention of one or more of a vascular condition, diabetes, obesity, metabolic syndrome, or lowering a concentration of a sterol in the plasma of a subject, comprising a therapeutically effective amount of a composition or therapeutic combination comprising: (a) at least one compound of Formula (I), or a pharmaceutically acceptable salt, solvate, or isomer ester thereof; (b) a cholesterol lowering compound; and (c) a pharmaceutically acceptable carrier.

As used herein, “therapeutic combination” or “combination therapy” means the administration of two or more therapeutic agents, such as a compound according to Formula (I) of the present invention, and a cholesterol lowering compound such as one or more substituted azetidinone or one or more substituted β-lactam, to prevent or treat a condition, for example a vascular condition, such as hyperlipidaemia (for example atherosclerosis, hypercholesterolemia or sitosterolemia), vascular inflammation, metabolic syndrome, stroke, diabetes, obesity and/or reduce the level of sterol(s) (such as cholesterol) in the plasma or tissue. As used herein, “vascular” comprises cardiovascular, cerebrovascular and combinations thereof. The compositions, combinations and treatments of the present invention can be administered by any suitable means which produce contact of these compounds with the site of action in the body, for example in the plasma, liver, small intestine, or brain (e.g., hippocampus, cortex, cerebellum, and basal ganglia) of a patient. Such administration includes co-administration of these therapeutic agents in a substantially simultaneous manner, such as in a single tablet or capsule having a fixed ratio of active ingredients or in multiple, separate capsules for each therapeutic agent. Also, such administration includes the administration of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complimentary effects or complimentary modes of action.

As discussed above, the compositions, pharmaceutical compositions and therapeutic combinations of the present invention comprise: (a) one or more compounds according to Formula (I) of the present invention, or pharmaceutically acceptable salts, solvates, isomers or esters thereof; and (b) one or more cholesterol lowering agents. A non-limiting list of cholesterol lowering agents useful in the present invention include HMG CoA reductase inhibitor compounds such as lovastatin (for example MEVACOR® which is available from Merck & Co.), simvastatin (for example ZOCOR® which is available from Merck & Co.), pravastatin (for example PRAVACHOL® which is available from Bristol Meyers Squibb), atorvastatin, fluvastatin (for example LESCOL®), cerivastatin, CI-981, rivastatin (sodium 7-(4-fluorophenyl)-2,6-diisopropyl-5-methoxymethylpyridin-3-yl)-3,5-dihydroxy-6-heptanoate), rosuvastatin calcium (CRESTOR® from AstraZeneca Pharmaceuticals), Pravastatin (marketed as LIVALO®), cerivastatin, itavastatin (or pitavastatin, NK-104 of Negma Kowa of Japan); HMG CoA synthetase inhibitors, for example L-659,699 ((E,E)-1143′R-(hydroxy-methyl)-4′-oxo-2′R-oxetanyl]-3,5,7R-trimethyl-2,4-undecadienoic acid); squalene synthesis inhibitors, for example squalestatin 1; squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride); sterol (e.g., cholesterol) biosynthesis inhibitors such as DMP-565; nicotinic acid derivatives (e.g., compounds comprising a pyridine-3-carboxylate structure or a pyrazine-2-carboxylate structure, including acid forms, salts, esters, zwitterions and tautomers) such as niceritrol, nicofuranose and acipimox (5-methylpyrazine-2-carboxylic acid 4-oxide), and niacin extended-release tablets such as NIASPAN®; clofibrate; gemfibrazol; bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and mixtures thereof; inorganic cholesterol sequestrants such as bismuth salicylate plus montmorillonite clay, aluminum hydroxide and calcium carbonate antacids; ileal bile acid transport (“IBAT”) inhibitors (or apical sodium co-dependent bile acid transport (“ASBT”) inhibitors) such as benzothiepines, for example the therapeutic compounds comprising a 2,3,4,5-tetrahydro-1-benzothiepine 1,1-dioxide structure such as are disclosed in PCT Patent Application WO 00/38727 which is incorporated herein by reference; AcylCoA:Cholesterol O-acyltransferase (“ACAT”) Inhibitors such as avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea), and the compounds described in P. Chang et al., “Current, New and Future Treatments in Dyslipidaemia and Atherosclerosis”, Drugs 2000 July; 60(1); 55-93, which is incorporated by reference herein; Cholesteryl Ester Transfer Protein (“CETP”) Inhibitors such as those disclosed in PCT Patent Application No. WO 00/38721 and U.S. Pat. No. 6,147,090, which are incorporated herein by reference; probucol or derivatives thereof, such as AGI-1067 and other derivatives disclosed in U.S. Pat. Nos. 6,121,319 and 6,147,250, herein incorporated by reference; low-density lipoprotein (LDL) receptor activators such as HOE-402, an imidazolidinyl-pyrimidine derivative that directly stimulates LDL receptor activity, described in M. Huettinger et al., “Hypolipidemic activity of HOE-402 is Mediated by Stimulation of the LDL Receptor Pathway”, Arterioscler. Thromb. 1993; 13: 1005-12, herein incorporated by reference; fish oils containing Omega 3 fatty acids (3-PUFA); natural water soluble fibers, such as psyllium, guar, oat and pectin; plant stanols and/or fatty acid esters of plant stanols, such as sitostanol ester used in BENECOL® margarine; nicotinic acid receptor agonists (e.g., agonists of the HM74 and HM74A receptor which receptor is described in US 2004/0142377, US 2005/0004178, US 2005/0154029, U.S. Pat. No. 6,902,902, WO 2004/071378, WO 2004/071394, WO 01/77320, US 2003/0139343, WO 01/94385, WO 2004/083388, US 2004/254224, US 2004/0254224, US 2003/0109673 and WO 98/56820) for example those described in WO 2004/033431, WO 2005/011677, WO 2005/051937, US 2005/0187280, US 2005/0187263, WO 2005/077950, WO 2005/016867, WO 2005/016870, WO2005061495, WO2006005195, WO2007059203, US2007105961, CA2574987, and AU2007200621; and the substituted azetidinone or substituted p-lactam sterol absorption inhibitors discussed in detail below.

As used herein, “sterol absorption inhibitor” means a compound capable of inhibiting the absorption of one or more sterols, including but not limited to cholesterol, phytosterols (such as sitosterol, campesterol, stigmasterol and avenosterol), 5α-stanols (such as cholestanol, 5α-campestanol, 5α-sitostanol), and/or mixtures thereof, when administered in a therapeutically effective (sterol and/or 5α-stanol absorption inhibiting) amount to a patient such as a mammal or human. Non-limiting examples of stanol absorption inhibitors include those compounds that inhibit cholesterol absorption in the small intestine. Such compounds are well known in the art and are described, for example, in US RE 37,721; U.S. Pat. No. 5,631,356; U.S. Pat. No. 5,767,115; U.S. Pat. No. 5,846,966; U.S. Pat. No. 5,698,548; U.S. Pat. No. 5,633,246; U.S. Pat. No. 5,656,624; U.S. Pat. No. 5,624,920; U.S. Pat. No. 5,688,787; U.S. Pat. No. 5,756,470; US Publication No. 2002/0137689; WO 02/066464; WO 95/08522 and WO96/19450. Non-limiting examples of cholesterol absorption inhibitors also include non-small molecule agents, microorganisms such as Bifidobacterium animalis subsp. animalis YIT 10394, Bifidobacterium animalis subsp. lactis JCM 1253, Bifidobacterium animalis subsp. lactis JCM 7117 and Bifidobacterium pseudolongum subsp. Globosum, which are described, e.g., in WO2007029773. Each of the aforementioned publications is incorporated by reference. Substituted Azetidinones of Formula (II)

In one embodiment, substituted azetidinones useful in the compositions, therapeutic combinations and methods of the present invention are represented by Formula (II) below:

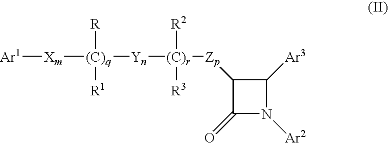

or pharmaceutically acceptable salts, solvates, or esters of the compounds of Formula (II), wherein, in Formula (II) above:

Ar¹ and Ar² are independently selected from the group consisting of aryl and R⁴-substituted aryl;

Ar³ is aryl or R⁵-substituted aryl;

X, Y and Z are independently selected from the group consisting of —CH₂—, —CH(lower alkyl)- and —C(lower alkyl)₂-;

R and R² are independently selected from the group consisting of —OR⁶, —OC(O)R⁶, —OC(O)OR⁹ and —OC(O)NR⁶R⁷;

R¹ and R³ are independently selected from the group consisting of hydrogen, lower alkyl and aryl;

q is 0 or 1; r is 0 or 1; m, n and p are independently selected from 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶SO₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀CONR⁶R⁷, -(lower alkylene)COOR⁶, —CH═CH—C(O)OR⁶, —CF₃, —CN, —NO₂ and halogen;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —SO₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl; and

R⁹ is lower alkyl, aryl or aryl-substituted lower alkyl.

Preferably, R⁴ is 1-3 independently selected substituents, and R⁵ is preferably 1-3 independently selected substituents.

Certain compounds useful in the therapeutic compositions or combinations of the invention may have at least one asymmetrical carbon atom and therefore all isomers, including enantiomers, diastereomers, stereoisomers, rotamers, tautomers and racemates of the compounds of Formula II-XIII (where they exist) are contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the Formulae II-XIII. Isomers may also include geometric isomers, e.g., when a double bond is present.

Those skilled in the art will appreciate that for some of the compounds of the Formulae II-XIII, one isomer may show greater pharmacological activity than other isomers.

Preferred compounds of Formula (II) are those in which Ar¹ is phenyl or R⁴-substituted phenyl, more preferably (4-R⁴)-substituted phenyl. Ar² is preferably phenyl or R⁴-substituted phenyl, more preferably (4-R⁴)-substituted phenyl. Ar³ is preferably R⁵-substituted phenyl, more preferably (4-R⁵)-substituted phenyl. When Ar¹ is (4-R⁴)-substituted phenyl, R⁴ is preferably a halogen. When Ar² and Ar³ are R⁴- and R⁵-substituted phenyl, respectively, R⁴ is preferably halogen or —OR⁶ and R⁵ is preferably —OR⁶, wherein R⁶ is lower alkyl or hydrogen. Especially preferred are compounds wherein each of Ar¹ and Ar² is 4-fluorophenyl and Ar³ is 4-hydroxyphenyl or 4-methoxyphenyl.

X, Y and Z are each preferably —CH₂—. R¹ and R³ are each preferably hydrogen. R and R² are preferably —OR⁶ wherein R⁶ is hydrogen, or a group readily metabolizable to a hydroxyl (such as —OC(O)R⁶, —OC(O)OR⁹ and —OC(O)NR⁶R⁷, defined above).

The sum of m, n, p, q and r is preferably 2, 3 or 4, more preferably 3. Preferred are compounds OF Formula (II) wherein m, n and r are each zero, q is 1 and p is 2.

Also preferred are compounds of Formula (II) in which p, q and n are each zero, r is 1 and m is 2 or 3. More preferred are compounds wherein m, n and r are each zero, q is 1, p is 2, Z is —CH₂— and R is —OR⁶, especially when R⁶ is hydrogen.

Also more preferred are compounds of Formula (II) wherein p, q and n are each zero, r is 1, m is 2, X is —CH₂— and R² is —OR⁶, especially when R⁶ is hydrogen.

Another group of preferred compounds of Formula (II) is that in which Ar¹ is phenyl or R⁴-substituted phenyl, Ar² is phenyl or R⁴-substituted phenyl and Ar³ is R⁵-substituted phenyl. Also preferred are compounds in which Ar¹ is phenyl or R⁴-substituted phenyl, Ar² is phenyl or R⁴-substituted phenyl, Ar³ is R⁵-substituted phenyl, and the sum of m, n, p, q and r is 2, 3 or 4, more preferably 3. More preferred are compounds wherein Ar¹ is phenyl or R⁴-substituted phenyl, Ar² is phenyl or R⁴-substituted phenyl, Ar³ is R⁵-substituted phenyl, and wherein m, n and r are each zero, q is 1 and p is 2, or wherein p, q and n are each zero, r is 1 and m is 2 or 3.

### Substituted Azetidinones of Formula (III)

In a preferred embodiment, a substituted azetidinone of Formula (II) useful in the compositions, therapeutic combinations and methods of the present invention is represented by Formula (III) (ezetimibe) below:

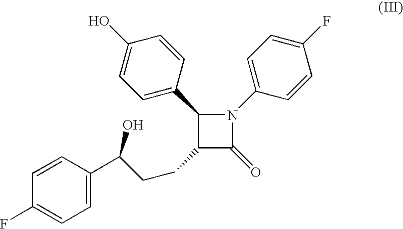

or pharmaceutically acceptable salts, solvates, or esters of the compound of Formula (III). The compound of Formula (III) can be in anhydrous or hydrated form. A product containing ezetimibe compound is commercially available as ZETIA® ezetimibe formulation from MSP Pharmaceuticals.

Compounds of Formula (II) can be prepared by a variety of methods well known to those skilled in the art, for example such as are disclosed in U.S. Pat. Nos. 5,631,365, 5,767,115, 5,846,966, 6,207,822, 6,627,757, 6,093,812, 5,306,817, 5,561,227, 5,688,785, and 5,688,787, each of which is incorporated herein by reference.

### Substituted Azetidinones of Formula (IV)

Alternative substituted azetidinones useful in the compositions, therapeutic combinations and methods of the present invention are represented by Formula (IV) below:

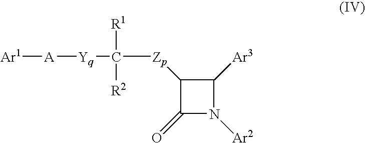

or a pharmaceutically acceptable salt thereof or a solvate thereof, or an ester thereof, wherein, in Formula (IV) above:

Ar¹ is R³-substituted aryl;

Ar² is R⁴-substituted aryl;

Ar³ is R⁵-substituted aryl;

Y and Z are independently selected from the group consisting of —CH₂—, —CH(lower alkyl)- and —C(lower alkyl)₂-;

A is selected from —O—, —S—, —S(O)— or —S(O)₂—;

R¹ is selected from the group consisting of —OR⁶, —OC(O)R⁶, —OC(O)OR⁹ and —OC(O)NR⁶R⁷;

R² is selected from the group consisting of hydrogen, lower alkyl and aryl; or R¹ and R² together are ═O;

q is 1, 2 or 3;

p is 0, 1, 2, 3 or 4;

R⁵ is 1-3 substituents independently selected from the group consisting of —OR⁶, —OC(O)R⁶, —OC(O)OR⁹, —O(CH₂)₁₋₅OR⁹, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂-lower alkyl, —NR⁶S(O)₂-aryl, —C(O)NR⁶R⁷, —COR^{E}, —SO₂NR⁶R⁷, S(O)₀₋₂-alkyl, S(O)₀₋₂-aryl, —O(CH₂)₁₋₁₀—C(O)OR⁶, —O(CH₂)₁₋₁₀C(O)NR⁶R⁷, o-halogeno, m-halogeno, o-lower alkyl, m-lower alkyl, -(lower alkylene)-C(O)OR⁶, and —CH═CH—C(O)OR⁶;

R³ and R⁴ are independently 1-3 substituents independently selected from the group consisting of R⁵, hydrogen, p-lower alkyl, aryl, —NO₂, —CF₃ and p-halogeno;

R⁶, R⁷ and R⁸ are independently selected from the group consisting of hydrogen, lower alkyl, aryl and aryl-substituted lower alkyl; and R⁹ is lower alkyl, aryl or aryl-substituted lower alkyl.

Methods for making compounds of Formula (IV) are well known to those skilled in the art. Non-limiting examples of suitable methods are disclosed in U.S. Pat. No. 5,688,990, which is incorporated herein by reference.

### Substituted Azetidinones of Formula (V)

In another embodiment, substituted azetidinones useful in the compositions, therapeutic combinations and methods of the present invention are represented by Formula (V):

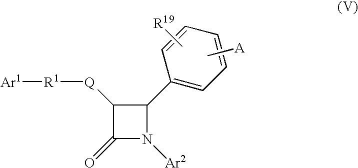

or a pharmaceutically acceptable salt thereof or a solvate thereof, or an ester thereof, wherein, in Formula (V) above:

A is selected from the group consisting of R²-substituted heterocycloalkyl, R²-substituted heteroaryl, R²-substituted benzo-fused heterocycloalkyl, and R²-substituted benzo-fused heteroaryl;

Ar¹ is aryl or R³-substituted aryl;

Ar² is aryl or R⁴-substituted aryl;

Q is a bond or, with the 3-position ring carbon of the azetidinone, forms the spiro group

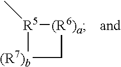

R¹ is selected from the group consisting of:
—(CH₂)_{q}—, wherein q is 2-6, provided that when Q forms a spiro ring, q can also be zero or 1;
—(CH₂)ₑ-G-(CH₂)ᵣ—, wherein G is —O—, —C(O)—, phenylene, —NR⁸— or —S(O)₀₋₂—, e is 0-5 and r is 0-5, provided that the sum of e and r is 1-6;
—(C₂-C₆ alkenylene)-; and
—(CH₂)_{f}—V—(CH₂)_{g}—, wherein V is C₃-C₆ cycloalkylene, f is 1-5 and g is 0-5, provided that the sum of f and g is 1-6;

R⁵ is selected from:

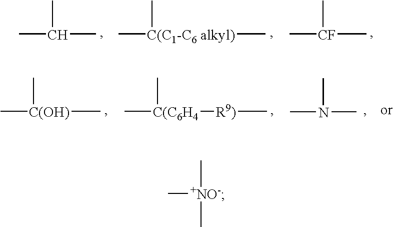

R⁶ and R⁷ are independently selected from the group consisting of —CH₂—, —CH(C₁-C₆ alkyl)-, —C(di-(C₁-C₆) alkyl), —CH═CH— and —C(C₁-C₆ alkyl)=CH—; or R⁵ together with an adjacent R⁶, or R⁵ together with an adjacent R⁷, form a —CH═CH— or a —CH═C(C₁-C₆ alkyl)- group;

a and b are independently 0, 1, 2 or 3, provided both are not zero; provided that when R⁶ is —CH═CH— or —C(C₁-C₆ alkyl)=CH—, a is 1; provided that when R⁷ is —CH═CH— or —C(C₁-C₆ alkyl)=CH—, b is 1; provided that when a is 2 or 3, the R⁶'s can be the same or different; and provided that when b is 2 or 3, the R⁷'s can be the same or different;

and when Q is a bond, R¹ also can be selected from:

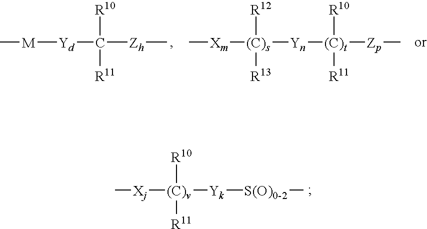

where M is —O—, —S—, —S(O)— or —S(O)₂—;

X, Y and Z are independently selected from the group consisting of —CH₂—, —CH(C₁-C₆ alkyl)- and —C(di-(C₁-C₆) alkyl);

R¹⁰ and R¹² are independently selected from the group consisting of —OR¹⁴, —OC(O)R¹⁴, —OC(O)OR¹⁶ and —OC(O)NR¹⁴R¹⁵;

R¹¹ and R¹³ are independently selected from the group consisting of hydrogen, (C₁-C₆)alkyl and aryl; or R¹⁰ and R¹¹ together are ═O, or R¹² and R¹³ together are ═O;

d is 1, 2 or 3;

h is 0, 1, 2, 3 or 4;

s is 0 or 1; t is 0 or 1; m, n and p are independently 0-4; provided that at least one of s and t is 1, and the sum of m, n, p, s and t is 1-6; provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

R² is 1-3 substituents on the ring carbon atoms selected from the group consisting of hydrogen, (C₁-C₁₀)alkyl, (C₂-C₁₀)alkenyl, (C₂-C₁₀)alkynyl, (C₃-C₆)cycloalkyl, (C₃-C₆)cycloalkenyl, R¹⁷-substituted aryl, R¹⁷-substituted benzyl, R¹⁷-substituted benzyloxy, R¹⁷-substituted aryloxy, halogeno, —NR¹⁴R¹⁵, NR¹⁴R¹⁵(C₁-C₆ alkylene)-, NR¹⁴R¹⁵C(O)(C₁-C₆ alkylene)-, —NHC(O)R¹⁶, OH, C₁-C₆ alkoxy, —OC(O)R¹⁶, —C(O)R¹⁴, hydroxy(C₁-C₆)alkyl, (C₁-C₆)alkoxy(C₁-C₆)alkyl, NO₂, —S(O)₀₋₂R¹⁶, —S(O)₂NR¹⁴R¹⁵ and —(C₁-C₆ alkylene)C(O)OR¹⁴; when R² is a substituent on a heterocycloalkyl ring, R² is as defined, or R² is ═O or

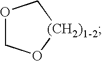

and, where R² is a substituent on a substitutable ring nitrogen, R² is hydrogen, (C₁-C₆)alkyl, aryl, (C₁-C₆)alkoxy, aryloxy, (C₁-C₆)alkylcarbonyl, arylcarbonyl, hydroxy, —(CH₂)₁₋₆CONR¹⁸R¹⁸,

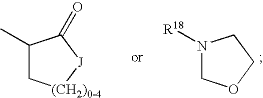

wherein J is —O—, —NH—, —NR¹⁸— or —CH₂—;

R³ and R⁴ are independently selected from the group consisting of 1-3 substituents independently selected from the group consisting of (C₁-C₆)alkyl, —OR¹⁴, —OC(O)R¹⁴, —OC(O)OR¹⁶, —O(CH₂)₁₋₅OR¹⁴, —OC(O)NR¹⁴R¹⁵, —NR¹⁴R¹⁵, —NR¹⁴C(O)R¹⁵, —NR¹⁴C(O)OR¹⁶, —NR¹⁴C(O)NR¹⁵R¹⁹, —NR¹⁴S(O)₂R¹⁶, —C(O)OR¹⁴, —C(O)NR¹⁴R¹⁵, —C(O)R¹⁴, —S(O)₂NR¹⁴R¹⁵, S(O)₀₋₂R¹⁶, —O(CH₂)₁₋₁₀—C(O)OR¹⁴, —O(CH₂)₁₋₁₀C(O)NR¹⁴R¹⁵, —(C₁-C₆ alkylene)-C(O)OR¹⁴, —CH═CH—C(O)OR¹⁴, —CF₃, —CN, —NO₂ and halogen;

R⁸ is hydrogen, (C₁-C₆)alkyl, aryl (C₁-C₆)alkyl, —C(O)R¹⁴ or —C(O)OR¹⁴;

R⁹ and R¹⁷ are independently 1-3 groups independently selected from the group consisting of hydrogen, (C₁-C₆)alkyl, (C₁-C₆)alkoxy, —C(O)OH, NO₂, —NR¹⁴R¹⁵, OH and halogeno;

R¹⁴ and R¹⁵ are independently selected from the group consisting of hydrogen, (C₁-C₆)alkyl, aryl and aryl-substituted (C₁-C₆)alkyl;

R¹⁶ is (C₁-C₆)alkyl, aryl or R¹⁷-substituted aryl;

R¹⁸ is hydrogen or (C₁-C₆)alkyl; and

R¹⁹ is hydrogen, hydroxy or (C₁-C₆)alkoxy.

Methods for making compounds of Formula (V) are well known to those skilled in the art. Non-limiting examples of suitable methods are disclosed in U.S. Pat. No. 5,656,624, which is incorporated herein by reference.

### Substituted Azetidinones of Formula (VI)

In another embodiment, substituted azetidinones useful in the compositions, therapeutic combinations and methods of the present invention are represented by Formula (VI):

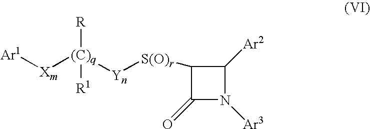

or a pharmaceutically acceptable salt thereof or a solvate thereof, or an ester thereof, wherein, in Formula (VI) above:

Ar¹ is aryl, R¹⁰-substituted aryl or heteroaryl;

Ar² is aryl or R⁴-substituted aryl;

Ar³ is aryl or R⁵-substituted aryl;

X and Y are independently selected from the group consisting of —CH₂—, —CH(lower alkyl)- and —C(lower alkyl)₂-;

R is —OR⁶, —OC(O)R⁶, —OC(O)OR⁹ or —OC(O)NR⁶R⁷; R¹ is hydrogen, lower alkyl or aryl; or R and R¹ together are ═O;

q is 0 or 1;

r is 0, 1 or 2;

m and n are independently 0, 1, 2, 3, 4 or 5; provided that the sum of m, n and q is 1, 2, 3, 4 or 5;

R⁴ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O)NR⁶R⁷, -(lower alkylene)C(O)OR⁶ and —CH═CH—C(O)OR⁶;

R⁵ is 1-5 substituents independently selected from the group consisting of —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, S(O)₀₋₂R⁹, —O(CH₂)₁₋₁₀—C(O)OR⁶, —O(CH₂)₁₋₁₀C(O)NR⁶R⁷, —CF₃, —CN, —NO₂, halogen, -(lower alkylene)C(O)OR⁶ and —CH═CH—C(O)OR⁶;

R⁶, R⁷ and R⁸ are independently selected from the group consisting of hydrogen, lower alkyl, aryl and aryl-substituted lower alkyl;

R⁹ is lower alkyl, aryl or aryl-substituted lower alkyl; and

R¹⁰ is 1-5 substituents independently selected from the group consisting of lower alkyl, —OR⁶, —OC(O)R⁶, —OC(O)OR⁹, —O(CH₂)₁₋₅OR⁶, —OC(O)NR⁶R⁷, —NR⁶R⁷, —NR⁶C(O)R⁷, —NR⁶C(O)OR⁹, —NR⁶C(O)NR⁷R⁸, —NR⁶S(O)₂R⁹, —C(O)OR⁶, —C(O)NR⁶R⁷, —C(O)R⁶, —S(O)₂NR⁶R⁷, —S(O)₀₋₂R⁹, —O(CH₂)₁₋₁₀—C(O)OR⁶, —O(CH₂)₁₋₁₀C(O)NR⁶R⁷, —CF₃, —CN, —NO₂ and halogen.

Methods for making compounds of Formula (VI) are well known to those skilled in the art. Non-limiting examples of suitable methods are disclosed in U.S. Pat. No. 5,624,920, which is incorporated herein by reference.

### Substituted Azetidinones of Formula (VII)

In another embodiment, substituted azetidinones useful in the compositions, therapeutic combinations and methods of the present invention are represented by Formula (VII):

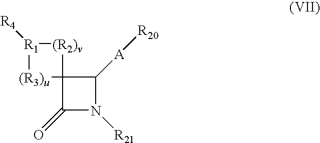

or a pharmaceutically acceptable salt thereof or a solvate thereof, or an ester thereof, wherein:

R¹ is:

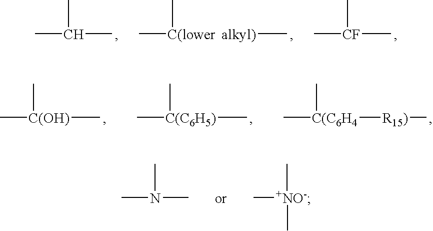

R² and R³ are independently selected from the group consisting of: —CH₂—, —CH(lower alkyl)-, —C(lower alkyl)₂-, —CH═CH— and —C(lower alkyl)=CH—; or R¹ together with an adjacent R², or R¹ together with an adjacent R³, form a —CH═CH— or a —CH═C(lower alkyl)- group;

u and v are independently 0, 1, 2 or 3, provided both are not zero; provided that when R² is —CH═CH— or —C(lower alkyl)=CH—, v is 1; provided that when R³ is —CH═CH— or —C(lower alkyl)=CH—, u is 1; provided that when v is 2 or 3, each R² can be the same or different; and provided that when u is 2 or 3, each R³ can be the same or different;

R⁴ is selected from B—(CH₂)ₘC(O)—, wherein m is 0, 1, 2, 3, 4 or 5; B—(CH₂)_{q}—, wherein q is 0, 1, 2, 3, 4, 5 or 6; B—(CH₂)ₑ—Z—(CH₂)ᵣ—, wherein Z is —O—, —C(O)—, phenylene, —N(R⁸)— or —S(O)₀₋₂—, e is 0, 1, 2, 3, 4 or 5 and r is 0, 1, 2, 3, 4 or 5, provided that the sum of e and r is 0, 1, 2, 3, 4, 5 or 6; B—(C₂-C₆ alkenylene)-; B—(C₄-C₆ alkadienylene)-; B—(CH₂)ᵣZ-(C₂-C₆ alkenylene)-, wherein Z is as defined above, and wherein t is 0, 1, 2 or 3, provided that the sum of t and the number of carbon atoms in the alkenylene chain is 2, 3, 4, 5 or 6; B—(CH₂)_{f}—V—(CH₂)_{g}—, wherein V is C₃-C₆ cycloalkylene, f is 1, 2, 3, 4 or 5 and g is 0, 1, 2, 3, 4 or 5, provided that the sum of f and g is 1, 2, 3, 4, 5 or 6; B—(CH₂)ₜ—V—(C₂-C₆ alkenylene)- or B—(C₂-C₆ alkenylene)-V—(CH₂)ₜ—, wherein V and t are as defined above, provided that the sum of t and the number of carbon atoms in the alkenylene chain is 2, 3, 4, 5 or 6; B—(CH₂)ₐ—Z—(CH₂)_{b}—V—(CH₂)_{d}—, wherein Z and V are as defined above and a, b and d are independently 0, 1, 2, 3, 4, 5 or 6, provided that the sum of a, b and d is 0, 1, 2, 3, 4, 5 or 6; or T-(CH₂)ₛ—, wherein T is a C₃-C₆ cycloalkyl and s is 0, 1, 2, 3, 4, 5 or 6; or

R¹ and R⁴ together form the group

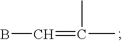

B is selected from indanyl, indenyl, naphthyl, tetrahydronaphthyl, heteroaryl or W-substituted heteroaryl, wherein heteroaryl is selected from the group consisting of pyrrolyl, pyridinyl, pyrimidinyl, pyrazinyl, triazinyl, imidazolyl, thiazolyl, pyrazolyl, thienyl, oxazolyl and furanyl, and for nitrogen-containing heteroaryls, the N-oxides thereof, or

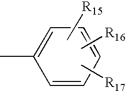

W is 1 to 3 substituents independently selected from the group consisting of lower alkyl, hydroxy lower alkyl, lower alkoxy, alkoxyalkyl, alkoxyalkoxy, alkoxycarbonylalkoxy, (lower alkoxyimino)-lower alkyl, lower alkanedioyl, lower alkyl lower alkanedioyl, allyloxy, —CF₃, —OCF₃, benzyl, R⁷-benzyl, benzyloxy, R⁷-benzyloxy, phenoxy, R⁷-phenoxy, dioxolanyl, NO₂, —N(R⁸)(R⁹), N(R⁸)(R⁹)-lower alkylene-, N(R⁸)(R⁹)-lower alkylenyloxy-, OH, halogeno, —CN, —N₃, —NHC(O)OR¹⁰, —NHC(O)R¹⁰, R¹¹(O)₂SNH—, (R¹¹(O)₂S)₂N—, —S(O)₂NH₂, —S(O)₀₋₂R⁸, tert-butyldimethyl-silyloxymethyl, —C(O)R¹², —C(O)OR¹⁹, —C(O)N(R⁸)(R⁹), —CH═CHC(O)R¹², -lower alkylene-C(O)R¹², R¹⁰C(O)(lower alkylenyloxy)-, N(R⁸)(R⁹)C(O)(lower alkylenyloxy)- and

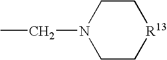

for substitution on ring carbon atoms, and the substituents on the substituted heteroaryl ring nitrogen atoms, when present, are selected from the group consisting of lower alkyl, lower alkoxy, —C(O)OR¹⁰, —C(O)R¹⁰, OH, N(R⁸)(R⁹)-lower alkylene-, N(R⁸)(R⁹)-lower alkylenyloxy-, —S(O)₂NH₂ and 2-(trimethylsilyl)-ethoxymethyl;

R⁷ is 1-3 groups independently selected from the group consisting of lower alkyl, lower alkoxy, —C(O)OH, NO₂, —N(R⁸)(R⁹), OH, and halogeno;

R⁸ and R⁹ are independently selected from H or lower alkyl;

R¹⁰ is selected from lower alkyl, phenyl, R⁷-phenyl, benzyl or R⁷-benzyl;

R¹¹ is selected from OH, lower alkyl, phenyl, benzyl, R⁷-phenyl or R⁷-benzyl;

R¹² is selected from H, OH, alkoxy, phenoxy, benzyloxy,

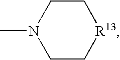

—N(R⁸)(R⁹), lower alkyl, phenyl or R⁷-phenyl;

R¹³ is selected from —O—, —CH₂—, —NH—, —N(lower alkyl)- or —NC(O)R¹⁹;

R¹⁵, R¹⁶ and R¹⁷ are independently selected from the group consisting of H and the groups defined for W; or R¹⁵ is hydrogen and R¹⁶ and R¹⁷, together with adjacent carbon atoms to which they are attached, form a dioxolanyl ring;

R¹⁹ is H, lower alkyl, phenyl or phenyl lower alkyl; and

R²⁰ and R²¹ are independently selected from the group consisting of phenyl, W-substituted phenyl, naphthyl, W-substituted naphthyl, indanyl, indenyl, tetrahydronaphthyl, benzodioxolyl, heteroaryl, W-substituted heteroaryl, benzo-fused heteroaryl, W-substituted benzo-fused heteroaryl and cyclopropyl, wherein heteroaryl is as defined above.

Methods for making compounds of Formula (VII) are well known to those skilled in the art. Non-limiting examples of suitable methods are disclosed in U.S. Pat. No. 5,698,548, which is incorporated herein by reference.

### Substituted Azetidinones of Formula (VIII)

In another embodiment, substituted azetidinones useful in the compositions, therapeutic combinations and methods of the present invention are represented by Formulas (VIIIA) and (VIIIB):

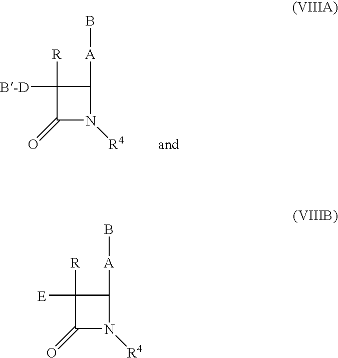

or a pharmaceutically acceptable salt, solvate, or ester thereof,
wherein:

A is —CH═CH—, —C≡C— or —(CH₂)ₚ— wherein p is 0, 1 or 2;

B is

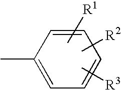

B′ is

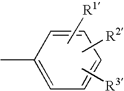

D is —(CH₂)ₘC(O)— or —(CH₂)_{q}— wherein m is 1, 2, 3 or 4 and q is 2, 3 or 4;

E is C₁₀ to C₂₀ alkyl or —C(O)—(C₉ to C₁₉)-alkyl, wherein the alkyl is straight or branched, saturated or containing one or more double bonds;

R is hydrogen, C₁-C₁₅ alkyl, straight or branched, saturated or containing one or more double bonds, or B—(CH₂)ᵣ—, wherein r is 0, 1, 2, or 3;

R¹, R², R³, R^{1,}, R^{2,}, and R^{3,} are independently selected from the group consisting of hydrogen, lower alkyl, lower alkoxy, carboxy, NO₂, NH₂, OH, halogeno, lower alkylamino, dilower alkylamino, —NHC(O)OR⁵, R⁶(O)₂SNH— and —S(O)₂NH₂,

R⁴ is

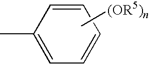

wherein n is 0, 1, 2 or 3;

R⁵ is lower alkyl; and

R⁶ is OH, lower alkyl, phenyl, benzyl or substituted phenyl wherein the substituents are 1-3 groups independently selected from the group consisting of lower alkyl, lower alkoxy, carboxy, NO₂, NH₂, OH, halogeno, lower alkylamino and dilower alkylamino; or a pharmaceutically acceptable salt, solvate, or ester thereof.

### Sterol Absorption Inhibitors of Formula (IX)

In another embodiment, sterol absorption inhibitors useful in the compositions and methods of the present invention are represented by Formula (IX):

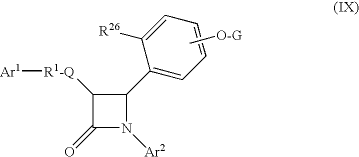

or a pharmaceutically acceptable salt, solvate, or ester thereof, wherein, in Formula (IX) above,

R²⁶ is H or OG¹;

G and G¹ are independently selected from the group consisting of H,

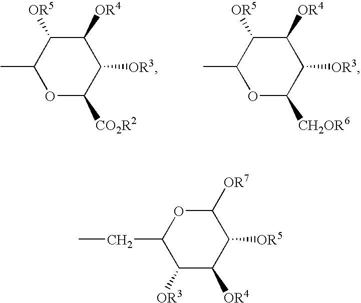

and

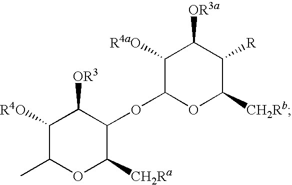

provided that when R²⁶ is H or OH, G is not H;

R, R^{a} and R^{b} are independently selected from the group consisting of H, —OH, halogeno, —NH₂, azido, (C₁-C₆)alkoxy(C₁-C₆)-alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are independently selected from the group consisting of H, (C₁-C₆)alkyl, aryl and aryl(C₁-C₆)alkyl;

R³, R⁴, R⁵, R⁷, R^{3a} and R^{4a} are independently selected from the group consisting of H, (C₁-C₆)alkyl, aryl(C₁-C₆)alkyl, —C(O)(C₁-C₆)alkyl and —C(O)aryl;

R³⁰ is selected from the group consisting of R³²-substituted T, R³²-substituted-T-(C₁-C₆)alkyl, R³²-substituted-(C₂-C₄)alkenyl,

R³²-substituted-(C₁-C₆)alkyl, R³²-substituted-(C₃-C₇)cycloalkyl and
R³²-substituted-(C₃-C₇)cycloalkyl(C₁-C₆)alkyl;

R³¹ is selected from the group consisting of H and (C₁-C₄)alkyl;

T is selected from the group consisting of phenyl, furyl, thienyl, pyrrolyl, oxazolyl, isoxazolyl, thiazolyl, iosthiazolyl, benzothiazolyl, thiadiazolyl, pyrazolyl, imidazolyl and pyridyl;

R³² is independently selected from 1-3 substituents independently selected from the group consisting of halogeno, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N((C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

Ar¹ is aryl or R¹⁰-substituted aryl;

Ar² is aryl or R¹¹-substituted aryl;

Q is a bond or, with the 3-position ring carbon of the azetidinone, forms the spiro group

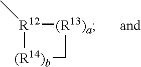

R¹ is selected from the group consisting of
—(CH₂)_{q}—, wherein q is 2-6, provided that when Q forms a spiro ring, q can also be zero or 1;
—(CH₂)ₑ-E-(CH₂)ᵣ, wherein E is —O—, —C(O)—, phenylene, —NR²²— or —S(O)₀₋₂—, e is 0-5 and r is 0-5, provided that the sum of e and r is 1-6;
—(C₂-C₆)alkenylene-; and
—(CH₂)_{f}—V—(CH₂)₉—, wherein V is C₃-C₆ cycloalkylene, f is 1-5 and g is 0-5, provided that the sum of f and g is 1-6;

R¹² is:

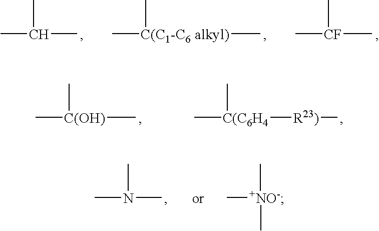

R¹³ and R¹⁴ are independently selected from the group consisting of —CH₂—, —CH((C₁-C₆) alkyl)-, —C((C₁-C₆) alkyl)₂, —CH═CH— and —C((C₁-C₆) alkyl)=CH—; or

R¹² together with an adjacent R¹³, or R¹² together with an adjacent R¹⁴, form a —CH═CH— or a —CH═C(C₁-C₆ alkyl)- group;

a and b are independently 0, 1, 2 or 3, provided both are not zero;

provided that when R¹³ is —CH═CH— or —C(C₁-C₆ alkyl)=CH—, a is 1;

provided that when R¹⁴ is —CH═CH— or —C(C₁-C₆ alkyl)=CH—, b is 1;

provided that when a is 2 or 3, each R¹³ can be the same or different; and

provided that when b is 2 or 3, each R¹⁴ can be the same or different; and when Q is a bond, R¹ also can be:

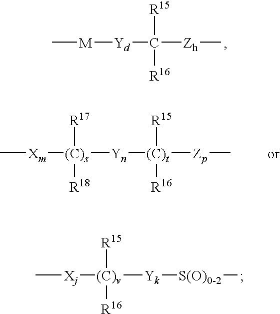

M is —O—, —S—, —S(O)— or —S(O)₂—;

X, Y and Z are independently selected from the group consisting of —CH₂—, —CH(C₁-C₆)alkyl- and —C((C₁-C₆)alkyl)₂;

R¹⁰ and R¹¹ are independently selected from the group consisting of 1-3 substituents independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR₁₉C(O)R²⁰, —NR¹⁹C(O)OR²¹, —NR¹⁹C(O)NR²⁰R²⁵, —NR¹⁹S(O)₂R²¹, —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halogen;

R¹⁵ and R¹⁷ are independently selected from the group consisting of —OR¹⁹, —OC(O)R¹⁹, —OC(O)OR²¹ and —OC(O)NR¹⁹R²⁰;

R¹⁶ and R¹⁸ are independently selected from the group consisting of H, (C₁-C₆)alkyl and aryl; or R¹⁵ and R¹⁶ together are ═O, or R¹⁷ and R¹⁸ together are ═O;

d is 1, 2 or 3;

h is 0, 1, 2, 3 or 4;

s is 0 or 1; t is 0 or 1; m, n and p are independently 0-4;

provided that at least one of s and t is 1, and the sum of m, n, p, s and t is 1-6;

provided that when p is 0 and t is 1, the sum of m, s and n is 1-5; and provided that when p is 0 and s is 1, the sum of m, t and n is 1-5;

v is 0 or 1;

j and k are independently 1-5, provided that the sum of j, k and v is 1-5;

and when Q is a bond and R¹ is

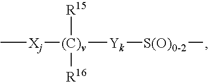

Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are independently selected from the group consisting of H, (C₁-C₆)alkyl, aryl and aryl-substituted (C₁-C₆)alkyl;

R²¹ is (C₁-C₆)alkyl, aryl or R²⁴-substituted aryl;

R²² is H, (C₁-C₆)alkyl, aryl (C₁-C₆)alkyl, —C(O)R¹⁹ or —C(O)OR¹⁹;

R²³ and R²⁴ are independently 1-3 groups independently selected from the group consisting of H, (C₁-C₆)alkyl, (C₁-C₆)alkoxy, —C(O)OH, NO₂, —NR¹⁹R²⁰, —OH and halogeno; and

R²⁵ is H, —OH or (C₁-C₆)alkoxy.

Methods for making compounds of Formula (IX) are well known to those skilled in the art. Non-limiting examples of suitable methods are disclosed in U.S. Pat. No. 5,756,470, which is incorporated herein by reference.

### Substituted Azetidinones of Formula (X)

In another embodiment, substituted azetidinones useful in the compositions and methods of the present invention are represented by Formula (X) below:

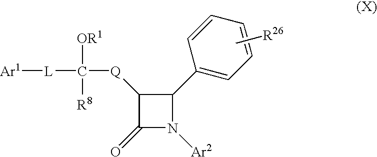

or a pharmaceutically acceptable salt, solvate, or ester thereof, wherein in Formula (X):

R¹ is selected from the group consisting of H, G, G¹, G², —SO₃H and —PO₃H;

G is selected from the group consisting of: H,

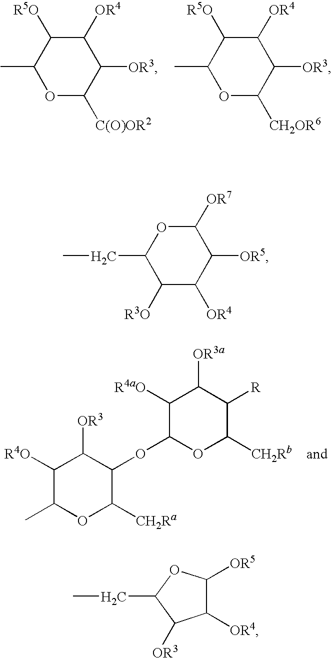

(sugar derivatives)

wherein R, R^{a} and R^{b} are each independently selected from the group consisting of H, —OH, halo, —NH₂, azido, (C₁-C₆)alkoxy(C₁-C₆)alkoxy or —W—R³⁰;

W is independently selected from the group consisting of —NH—C(O)—, —O—C(O)—, —O—C(O)—N(R³¹)—, —NH—C(O)—N(R³¹)— and —O—C(S)—N(R³¹)—;

R² and R⁶ are each independently selected from the group consisting of H, (C₁-C₆)alkyl, acetyl, aryl and aryl(C₁-C₆)alkyl;

R³, R⁴, R⁵, R⁷, R^{3a} and R^{4a} are each independently selected from the group consisting of H, (C₁-C₆)alkyl, acetyl, aryl(C₁-C₆)alkyl, —C(O)(C₁-C₆)alkyl and —C(O)aryl;

R³⁰ is independently selected from the group consisting of R³²-substituted T, R³²-substituted-T-(C₁-C₆)alkyl, R³²-substituted-(C₂-C₄)alkenyl, R³²-substituted-(C₁-C₆)alkyl, R³²-substituted-(C₃-C₇)cycloalkyl and R³²-substituted-(C₃-C₇)cycloalkyl(C₁-C₆)alkyl;

R³¹ is independently selected from the group consisting of H and (C₁-C₄)alkyl;

T is independently selected from the group consisting of phenyl, furyl, thienyl, pyrrolyl, oxazolyl, isoxazolyl, thiazolyl, isothiazolyl, benzothiazolyl, thiadiazolyl, pyrazolyl, imidazolyl and pyridyl;

R³² is independently selected from 1-3 substituents which are each independently selected from the group consisting of H, halo, (C₁-C₄)alkyl, —OH, phenoxy, —CF₃, —NO₂, (C₁-C₄)alkoxy, methylenedioxy, oxo, (C₁-C₄)alkylsulfanyl, (C₁-C₄)alkylsulfinyl, (C₁-C₄)alkylsulfonyl, —N(CH₃)₂, —C(O)—NH(C₁-C₄)alkyl, —C(O)—N(C₁-C₄)alkyl)₂, —C(O)—(C₁-C₄)alkyl, —C(O)—(C₁-C₄)alkoxy and pyrrolidinylcarbonyl; or

R³² is a covalent bond and R³¹, the nitrogen to which it is attached and R³² form a pyrrolidinyl, piperidinyl, N-methyl-piperazinyl, indolinyl or morpholinyl group, or a (C₁-C₄)alkoxycarbonyl-substituted pyrrolidinyl, piperidinyl, N-methylpiperazinyl, indolinyl or morpholinyl group;

G¹ is represented by the structure:

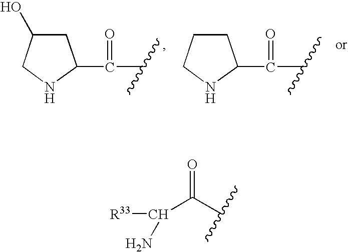

wherein R³³ is independently selected from the group consisting of unsubstituted alkyl, R³⁴-substituted alkyl, (R³⁵)(R³⁶)alkyl-,

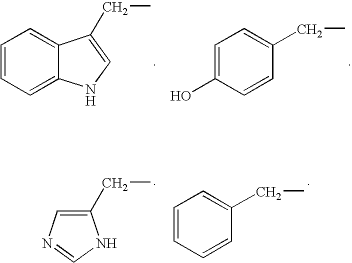

R³⁴ is one to three substituents, each R³⁴ being independently selected from the group consisting of HO(O)C—, HO—, HS—, (CH₃)S—, H₂N—, (NH₂)(NH)C(NH)—, (NH₂)C(O)— and HO(O)CCH(NH₃⁺)CH₂SS—;

R³⁵ is independently selected from the group consisting of H and NH₂—;

R³⁶ is independently selected from the group consisting of H, unsubstituted alkyl, R³⁴-substituted alkyl, unsubstituted cycloalkyl and R³⁴-substituted cycloalkyl;

G² is represented by the structure:

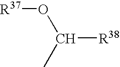

wherein R³⁷ and R³⁸ are each independently selected from the group consisting of (C₁-C₆)alkyl and aryl;

R²⁶ is one to five substituents, each R²⁶ being independently selected from the group consisting of:
a) H;
b) —OH;
c)—OCH₃;
d) fluorine;
e) chlorine;
f) —O-G;
g) —O-G¹;
h) —O-G²;
i) —SO₃H; and
j) —PO₃H;
   provided that when R¹ is H, R²⁶ is not H, —OH, —OCH₃ or —O-G;

Ar¹ is aryl, R¹⁰-substituted aryl, heteroaryl or R¹⁰-substituted heteroaryl;

Ar² is aryl, R¹¹-substituted aryl, heteroaryl or R¹¹-substituted heteroaryl;

L is selected from the group consisting of:
a) a covalent bond;
b) —(CH₂)_{q}—, wherein q is 1-6;
c) —(CH₂)ₑ-E-(CH₂)ᵣ—, wherein E is —O—, —C(O)—, phenylene, —NR²²— or —S(O)₀₋₂—, e is 0-5 and r is 0-5, provided that the sum of e and r is 1-6;
d) —(C₂-C₆)alkenylene-;
e) —(CH₂)_{f}—V—(CH₂)_{g}—, wherein V is C₃-C₆cycloalkylene, f is 1-5 and g is 0-5, provided that the sum of f and g is 1-6; and
f)

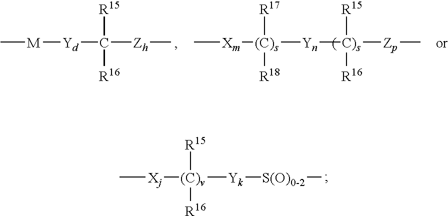

wherein M is —O—, —S—, —S(O)— or —S(O)₂—;

X, Y and Z are each independently selected from the group consisting of —CH₂—, —CH(C₁-C₆)alkyl- and —C((C₁-C₆)alkyl)₂-;

R⁸ is selected from the group consisting of H and alkyl;

R¹⁰ and R¹¹ are each independently selected from the group consisting of 1-3 substituents which are each independently selected from the group consisting of (C₁-C₆)alkyl, —OR¹⁹, —OC(O)R¹⁹, —OC(O)OR²¹, —O(CH₂)₁₋₅OR¹⁹, —OC(O)NR¹⁹R²⁰, —NR¹⁹R²⁰, —NR¹⁹C(O)R²⁰, —NR¹⁹C(O)OR²¹, —NR¹⁹C(O)NR²⁰R²⁵, —NR¹⁹S(O)₂R²¹, —C(O)OR¹⁹, —C(O)NR¹⁹R²⁰, —C(O)R¹⁹, —S(O)₂NR¹⁹R²⁰, S(O)₀₋₂R²¹, —O(CH₂)₁₋₁₀—C(O)OR¹⁹, —O(CH₂)₁₋₁₀C(O)NR¹⁹R²⁰, —(C₁-C₆ alkylene)-C(O)OR¹⁹, —CH═CH—C(O)OR¹⁹, —CF₃, —CN, —NO₂ and halo;

R¹⁵ and R¹⁷ are each independently selected from the group consisting of —OR¹⁹, —OC(O)R¹⁹, —OC(O)OR²¹, —OC(O)NR¹⁹R²⁰;

R¹⁶ and R¹⁸ are each independently selected from the group consisting of H, (C₁-C₆)alkyl and aryl; or

R¹⁵ and R¹⁶ together are ═O, or R¹⁷ and R¹⁸ together are ═O;

d is 1, 2 or 3;

h is 0, 1, 2, 3 or 4;

s is 0 or 1;

t is 0 or 1;

m, n and p are each independently selected from 0-4;

provided that at least one of s and t is 1, and the sum of m, n, p, s and t is 1-6; provided that when p is 0 and t is 1, the sum of m, n and p is 1-5; and provided that when p is 0 and s is 1, the sum of m, t and n is 1-5;

v is 0 or 1;

j and k are each independently 1-5, provided that the sum of j, k and v is 1-5;

Q is a bond, —(CH₂)_{q}—, wherein q is 1-6, or, with the 3-position ring carbon of the azetidinone, forms the spiro group

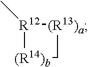

wherein R¹² is

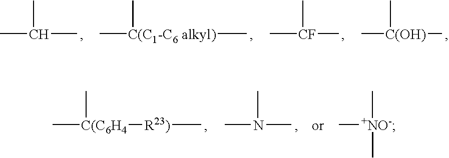

R¹³ and R¹⁴ are each independently selected from the group consisting of —CH₂—, —CH(C₁-C₆ alkyl)-, —C((C₁-C₆) alkyl)₂, —CH═CH— and —C(C₁-C₆ alkyl)=CH—; or R¹² together with an adjacent R¹³, or R¹² together with an adjacent R¹⁴, form a —CH═CH— or a —CH═C(C₁-C₆ alkyl)- group;

a and b are each independently 0, 1, 2 or 3, provided both are not zero; provided that when R¹³ is —CH═CH— or —C(C₁-C₆ alkyl)=CH—, a is 1; provided that when R¹⁴ is —CH═CH— or —C(C₁-C₆ alkyl)=CH—, b is 1; provided that when a is 2 or 3, each R¹³ can be the same or different; and provided that when b is 2 or 3, each R¹⁴ can be the same or different;

and when Q is a bond and L is

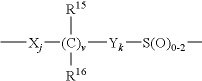

then Ar¹ can also be pyridyl, isoxazolyl, furanyl, pyrrolyl, thienyl, imidazolyl, pyrazolyl, thiazolyl, pyrazinyl, pyrimidinyl or pyridazinyl;

R¹⁹ and R²⁰ are each independently selected from the group consisting of H, (C₁-C₆)alkyl, aryl and aryl-substituted (C₁-C₆)alkyl;

R²¹ is (C₁-C₆)alkyl, aryl or R^{≧}-substituted aryl;

R²² is H, (C₁-C₆)alkyl, aryl (C₁-C₆)alkyl, —C(O)R¹⁹ or —C(O)OR¹⁹;

R²³ and R²⁴ are each independently selected from the group consisting of 1-3 substituents which are each independently selected from the group consisting of H, (C₁-C₆)alkyl, (C₁-C₆)alkoxy, —C(O)OH, NO₂, —NR¹⁹R²⁰, —OH and halo; and

R²⁵ is H, —OH or (C₁-C₆)alkoxy.

Examples of compounds of Formula (X) which are useful in the methods and combinations of the present invention and methods for making such compounds are disclosed in U.S. patent application Ser. No. 10/166,942, filed Jun. 11, 2002, incorporated herein by reference.

### Substituted Azetidinones of Formulae (XI)-(XIII)

An example of a useful substituted azetidinone is one represented by the Formula (XI):

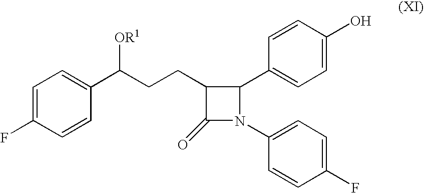

wherein R¹ is defined as above.

A more preferred compound is one represented by Formula (XII):

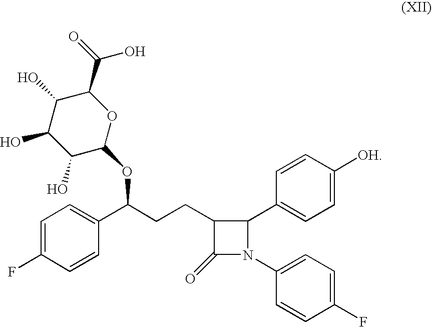

Another useful compound is represented by Formula (XIII):

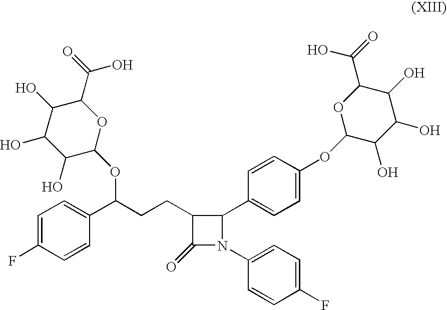

Other useful substituted azetidinone compounds include N-sulfonyl-2-azetidinones such as are disclosed in U.S. Pat. No. 4,983,597, ethyl 4-(2-oxoazetidin-4-yl)phenoxy-alkanoates such as are disclosed in Ram et al., Indian J. Chem. Sect. B. 29B, 12 (1990), p. 1134-7, diphenyl azetidinones and derivatives disclosed in U.S. Patent Publication Nos. 2002/0039774, 2002/0128252, 2002/0128253 and 2002/0137689, 2004/063929, WO 2002/066464, U.S. Pat. Nos. 6,498,156 and 6,703,386, each of which is incorporated by reference herein.

Other sterol absorption inhibitors useful in the compositions, therapeutic combinations and methods of the present invention are described in WO 2004/005247, WO 2004/000803, WO 2004/000804, WO 2004/000805, WO 0250027, U.S. published application 2002/0137689, and the compounds described in L. Kværnø et al., Angew. Chem. Int. Ed., 2004, vol. 43, pp. 4653-4656, all of which are incorporated herein by reference. An illustrative compound of Kv [symbol] rnø et al. is:

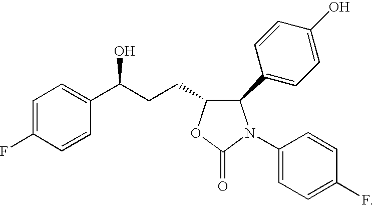

The compounds of Formulae II-XIII can be prepared by known methods, including the methods discussed above and, for example, in WO 93/02048, U.S. Pat. Nos. 5,306,817 and 5,561,227, herein incorporated by reference, which describe the preparation of compounds wherein —R¹-Q- is alkylene, alkenylene or alkylene interrupted by a hetero atom, phenylene or cycloalkylene; WO 94/17038 and U.S. Pat. No. 5,698,548, herein incorporated by reference, describe the preparation of compounds wherein Q is a spirocyclic group; WO 95/08532, U.S. Pat. No. 5,631,365, U.S. Pat. No. 5,767,115, U.S. Pat. No. 5,846,966, and U.S. R.E. 37,721, herein incorporated by reference, describe the preparation of compounds wherein —R¹-Q- is a hydroxy-substituted alkylene group; PCT/US95/03196, herein incorporated by reference, describes compounds wherein —R¹-Q- is a hydroxy-substituted alkylene attached to the Ar¹ moiety through an —O- or S(O)₀₋₂-group; and U.S. Ser. No. 08/463,619, filed Jun. 5, 1995, herein incorporated by reference, describes the preparation of compounds wherein —R¹-Q- a hydroxy-substituted alkylene group attached to the azetidinone ring by a —S(O)₀₋₂-group. Each of the above patents or publications are herein incorporated by reference in their entirety.

The daily dose of the sterol absorption inhibitor(s) administered to the subject can range from about 0.1 to about 1000 mg per day, preferably about 0.25 to about 50 mg/day, and more preferably about 10 mg per day, given in a single dose or 2-4 divided doses. The exact dose, however, is determined by the attending clinician and is dependent on the potency of the compound administered, the age, weight, condition and response of the patient.

For administration of pharmaceutically acceptable salts of the above compounds, the weights indicated above refer to the weight of the acid equivalent or the base equivalent of the therapeutic compound derived from the salt.

In another embodiment of the present invention, the compositions or therapeutic combinations described above comprise one or more selective CB₁ receptor antagonist compounds of Formula (I) in combination with one or more cholesterol biosynthesis inhibitors and/or lipid-lowering compounds discussed below.

Generally, a total daily dosage of cholesterol biosynthesis inhibitor(s) can range from about 0.1 to about 160 mg per day, and preferably about 0.2 to about 80 mg/day in single or 2-3 divided doses.

In another alternative embodiment, the compositions, therapeutic combinations or methods of the present invention can comprise at least one compound of Formula (I), or pharmaceutically acceptable salts, solvates, or esters thereof, and one or more bile acid sequestrants (insoluble anion exchange resins), co-administered with or in combination with the compound of Formula (I), or a pharmaceutically acceptable salt, solvate, isomer, or ester thereof, and a substituted azetidinone or a substituted β-lactam discussed above.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors that bind LDL from plasma to further reduce cholesterol levels in the blood.

Generally, a total daily dosage of bile acid sequestrant(s) can range from about 1 to about 50 grams per day, and preferably about 2 to about 16 grams per day in single or 2-4 divided doses.

In an alternative embodiment, the compositions or treatments of the present invention can comprise at least one compound of Formula (I), or pharmaceutically acceptable salts, solvates, or esters thereof, and one or more IBAT inhibitors. The IBAT inhibitors can inhibit bile acid transport to reduce LDL cholesterol levels. Generally, a total daily dosage of IBAT inhibitor(s) can range from about 0.01 to about 1000 mg/day, and preferably about 0.1 to about 50 mg/day in single or 2-4 divided doses.

In another alternative embodiment, the compositions or treatments of the present invention can comprise at least one compound of Formula (I), or pharmaceutically acceptable salts, solvates, or esters thereof, and nicotinic acid (niacin) and/or derivatives thereof. Nicotinic acid and its derivatives inhibit hepatic production of VLDL and its metabolite LDL and increases HDL and apo A-1 levels. An example of a suitable nicotinic acid product is NIASPAN® (niacin extended-release tablets), which are available from Kos.

Generally, a total daily dosage of nicotinic acid or a derivative thereof can range from about 500 to about 10,000 mg/day, preferably about 1000 to about 8000 mg/day, and more preferably about 3000 to about 6000 mg/day in single or divided doses.

In another alternative embodiment, the compositions or treatments of the present invention can comprise at least one compound of Formula (I), or pharmaceutically acceptable salts, solvates, or estes thereof, and one or more AcylCoA:Cholesterol O-acyltransferase (“ACAT”) Inhibitors, which can reduce LDL and VLDL levels. ACAT is an enzyme responsible for esterifying excess intracellular cholesterol and may reduce the synthesis of VLDL, which is a product of cholesterol esterification, and overproduction of apo B-100-containing lipoproteins. Generally, a total daily dosage of ACAT inhibitor(s) can range from about 0.1 to about 1000 mg/day in single or 2-4 divided doses.

In another alternative embodiment, the compositions or treatments of the present invention can comprise at least one compound of Formula (I), or pharmaceutically acceptable salts, solvates, or esters thereof, and one or more Cholesteryl Ester Transfer Protein (“CETP”) Inhibitors, such as torcetrapib. CETP is responsible for the exchange or transfer of cholesteryl ester carrying HDL and triglycerides in VLDL. Pancreatic cholesteryl ester hydrolase (pCEH) inhibitors such as WAY-121898 also can be co-administered with or in combination.

Generally, a total daily dosage of CETP inhibitor(s) can range from about 0.01 to about 1000 mg/day, and preferably about 0.5 to about 20 mg/kg body weight/day in single or divided doses.

In another alternative embodiment, the compositions or treatments of the present invention can comprise at least one compound of Formula (I), or pharmaceutically acceptable salts, solvates, or esters thereof, and probucol or derivatives thereof, which can reduce LDL levels.

Generally, a total daily dosage of probucol or derivatives thereof can range from about 10 to about 2000 mg/day, and preferably about 500 to about 1500 mg/day in single or 2-4 divided doses.

In another alternative embodiment, the compositions or treatments of the present invention can comprise at least one compound of Formula (I), or pharmaceutically acceptable salts, solvates, or esters thereof, and low-density lipoprotein (LDL) receptor activators.

Generally, a total daily dosage of LDL receptor activator(s) can range from about 1 to about 1000 mg/day in single or 2-4 divided doses.

In another alternative embodiment, the compositions or treatments of the present invention can comprise at least one compound of Formula (I), or pharmaceutically acceptable salts, solvates, or esters thereof, and fish oil. Generally, a total daily dosage of fish oil or Omega 3 fatty acids can range from about 1 to about 30 grams per day in single or 2-4 divided doses.

In another alternative embodiment, the compositions or treatments of the present invention can further comprise at least one compound of Formula (I), or pharmaceutically acceptable salts, solvates, or esters thereof, and natural water soluble fibers, such as psyllium, guar, oat and pectin, which can reduce cholesterol levels. Generally, a total daily dosage of natural water soluble fibers can range from about 0.1 to about 10 grams per day in single or 2-4 divided doses.

In another alternative embodiment, the compositions or treatments of the present invention can comprise at least one compound of Formula (I), or pharmaceutically acceptable salts, solvates, or esters thereof, and plant sterols, plant stanols and/or fatty acid esters of plant stanols, such as sitostanol ester used in BENECOL® margarine, which can reduce cholesterol levels. Generally, a total daily dosage of plant sterols, plant stanols and/or fatty acid esters of plant stanols can range from about 0.5 to about 20 grams per day in single or 2-4 divided doses.

In another alternative embodiment, the compositions or treatments of the present invention can comprise at least one compound of Formula (I), or pharmaceutically acceptable salts, solvates, or esters thereof, and antioxidants, such as probucol, tocopherol, ascorbic acid, β-carotene and selenium, or vitamins such as vitamin B₆ or vitamin B₁₂. Generally, a total daily dosage of antioxidants or vitamins can range from about 0.05 to about 10 grams per day in single or 2-4 divided doses.

In another alternative embodiment, the compositions or treatments of the present invention can comprise at least one compound of Formula (I), or pharmaceutically acceptable salts, solvates, or esters thereof, and monocyte and macrophage inhibitors such as polyunsaturated fatty acids (PUFA), thyroid hormones including throxine analogues such as CGS-26214 (a thyroxine compound with a fluorinated ring), gene therapy and use of recombinant proteins such as recombinant apo E. Generally, a total daily dosage of these agents can range from about 0.01 to about 1000 mg/day in single or 2-4 divided doses.

Also useful with the present invention are compositions or therapeutic combinations that further comprise hormone replacement agents and compositions. Useful hormone agents and compositions for hormone replacement therapy of the present invention include androgens, estrogens, progestins, their pharmaceutically acceptable salts and derivatives thereof. Combinations of these agents and compositions are also useful.

The dosage of androgen and estrogen combinations vary, desirably from about 1 mg to about 4 mg androgen and from about 1 mg to about 3 mg estrogen. Examples include, but are not limited to, androgen and estrogen combinations such as the combination of esterified estrogens (sodium estrone sulfate and sodium equilin sulfate) and methyltestosterone (17-hydroxy-17-methyl-, (17B)-androst-4-en-3-one) available from Solvay Pharmaceuticals, Inc., Marietta, Ga., under the tradename Estratest.

Estrogens and estrogen combinations may vary in dosage from about 0.01 mg up to 8 mg, desirably from about 0.3 mg to about 3.0 mg. Examples of useful estrogens and estrogen combinations include:

(a) the blend of nine (9) synthetic estrogenic substances including sodium estrone sulfate, sodium equilin sulfate, sodium 17 α-dihydroequilin sulfate, sodium 17 α-estradiol sulfate, sodium 17 β-dihydroequilin sulfate, sodium 17 α-dihydroequilenin sulfate, sodium 17 β-dihydroequilenin sulfate, sodium equilenin sulfate and sodium 17 β-estradiol sulfate; available from Duramed Pharmaceuticals, Inc., Cincinnati, Ohio, under the tradename Cenestin;

(b) ethinyl estradiol (19-nor-17 α-pregna-1,3,5(10)-trien-20-yne-3,17-diol; available by Schering Plough Corporation, Kenilworth, N.J., under the tradename Estinyl;

(c) esterified estrogen combinations such as sodium estrone sulfate and sodium equilin sulfate; available from Solvay under the tradename Estratab and from Monarch Pharmaceuticals, Bristol, Tenn., under the tradename Menest;

(d) estropipate (piperazine estra-1,3,5(10)-trien-17-one, 3-(sulfooxy)-estrone sulfate); available from Pharmacia & Upjohn, Peapack, N.J., under the tradename Ogen and from Women First Health Care, Inc., San Diego, Calif., under the tradename Ortho-Est; and

(e) conjugated estrogens (17 α-dihydroequilin, 17 α-estradiol, and 17 β-dihydroequilin); available from Wyeth-Ayerst Pharmaceuticals, Philadelphia, Pa., under the tradename Premarin.

Progestins and estrogens may also be administered with a variety of dosages, generally from about 0.05 to about 2.0 mg progestin and about 0.001 mg to about 2 mg estrogen, desirably from about 0.1 mg to about 1 mg progestin and about 0.01 mg to about 0.5 mg estrogen. Examples of progestin and estrogen combinations that may vary in dosage and regimen include:

(a) the combination of estradiol (estra-1,3,5 (10)-triene-3, 17 β-diol hemihydrate) and norethindrone (17 β-acetoxy-19-nor-17 α-pregn-4-en-20-yn-3-one); which is available from Pharmacia & Upjohn, Peapack, N.J., under the tradename Activella;

(b) the combination of levonorgestrel (d(−)-13 β-ethyl-17 α-ethinyl-17 α-hydroxygon-4-en-3-one) and ethinyl estradial; available from Wyeth-Ayerst under the tradename Alesse, from Watson Laboratories, Inc., Corona, Calif., under the tradenames Levora and Trivora, Monarch Pharmaceuticals, under the tradename Nordette, and from Wyeth-Ayerst under the tradename Triphasil;

(c) the combination of ethynodiol diacetate (19-nor-17 α-pregn-4-en-20-yne-3 β, 17-diol diacetate) and ethinyl estradiol; available from G. D. Searle & Co., Chicago, Ill., under the tradename Demulen and from Watson under the tradename Zovia;

(d) the combination of desogestrel (13-ethyl-11-methylene-18,19-dinor-17 α-pregn-4-en-20-yn-17-ol) and ethinyl estradiol; available from Organon under the tradenames Desogen and Mircette, and from Ortho-McNeil Pharmaceutical, Raritan, N.J., under the tradename Ortho-Cept;

(e) the combination of norethindrone and ethinyl estradiol; available from Parke-Davis, Morris Plains, N.J., under the tradenames Estrostep and FemHRT, from Watson under the tradenames Microgestin, Necon, and Tri-Norinyl, from Ortho-McNeil under the tradenames Modicon and Ortho-Novum, and from Warner Chilcott Laboratories, Rockaway, N.J., under the tradename Ovcon;

(f) the combination of norgestrel ((±)-13-ethyl-17-hydroxy-18,19-dinor-17 α-preg-4-en-20-yn-3-one) and ethinyl estradiol; available from Wyeth-Ayerst under the tradenames Ovral and Lo/Ovral, and from Watson under the tradenames Ogestrel and Low-Ogestrel;

(g) the combination of norethindrone, ethinyl estradiol, and mestranol (3-methoxy-19-nor-17 α-pregna-1,3,5(10)-trien-20-yn-17-ol); available from Watson under the tradenames Brevicon and Norinyl;

(h) the combination of 17 β-estradiol (estra-1,3,5(10)-triene-3,17 β-diol) and micronized norgestimate (17 α-17-(Acetyloxyl)-13-ethyl-18,19-dinorpregn-4-en-20-yn-3-one3-oxime); available from Ortho-McNeil under the tradename Ortho-Prefest;

(i) the combination of norgestimate (18,19-dinor-17-pregn-4-en-20-yn-3-one, 17-(acetyloxy)-13-ethyl-,oxime, (17(α)-(+)-) and ethinyl estradiol; available from Ortho-McNeil under the tradenames Ortho Cyclen and Ortho Tri-Cyclen; and

(j) the combination of conjugated estrogens (sodium estrone sulfate and sodium equilin sulfate) and medroxyprogesterone acetate (20-dione, 17-(acetyloxy)-6-methyl-, (6(α))- pregn-4-ene-3); available from Wyeth-Ayerst under the tradenames Premphase and Prempro.

In general, a dosage of progestins may vary from about 0.05 mg to about 10 mg or up to about 200 mg if microsized progesterone is administered. Examples of progestins include norethindrone; available from ESI Lederle, Inc., Philadelphia, Pa., under the tradename Aygestin, from Ortho-McNeil under the tradename Micronor, and from Watson under the tradename Nor-QD; norgestrel; available from Wyeth-Ayerst under the tradename Ovrette; micronized progesterone (pregn-4-ene-3,20-dione); available from Solvay under the tradename Prometrium; and medroxyprogesterone acetate; available from Pharmacia & Upjohn under the tradename Provera.

In another alternative embodiment, the compositions, therapeutic combinations or methods of the present invention can comprise at least one compound of Formula (I), or pharmaceutically acceptable salts, solvates, isomers or esters thereof, and one or more obesity control medications. Useful obesity control medications include, but are not limited to, drugs that reduce energy intake or suppress appetite, drugs that increase energy expenditure and nutrient-partitioning agents. Suitable obesity control medications include, but are not limited to, noradrenergic agents (such as diethylpropion, mazindol, phenylpropanolamine, phentermine, phendimetrazine, phendamine tartrate, methamphetamine, phendimetrazine and tartrate); serotonergic agents (such as sibutramine, fenfluramine, dexfenfluramine, fluoxetine, fluvoxamine and paroxtine); thermogenic agents (such as ephedrine, caffeine, theophylline, and selective β3-adrenergic agonists); alpha-blocking agents; kainite or AMPA receptor antagonists; leptin-lipolysis stimulated receptors; phosphodiesterase enzyme inhibitors (such as milrinoone, theophylline, vinpocetine, EHNA (erythro-9-(2-hydroxy-3-monyl)adenine), sildenafil citrate, marketed as VIAGRA®, and tadalafil, marketed as Clalis®); compounds having nucleotide sequences of the mahogany gene; fibroblast growth factor-10 polypeptides; monoamine oxidase inhibitors (such as befloxatone, moclobemide, brofaromine, phenoxathine, esuprone, befol, toloxatone, pirlindol, amiflamine, sercloremine, bazinaprine, lazabemide, milacemide and caroxazone); compounds for increasing lipid metabolism (such as evodiamine compounds); and lipase inhibitors (such as orlistat). Generally, a total dosage of the above-described obesity control medications can range from 1 to 3,000 mg/day, desirably from about 1 to 1,000 mg/day and more desirably from about 1 to 200 mg/day in single or 2-4 divided doses.

The compositions, therapeutic combinations or methods of the present invention can comprise at least one compound of Formula (I), or pharmaceutically acceptable salts, solvates, isomers or esters thereof, and one or more blood modifiers which are chemically different from the substituted azetidinone and substituted β-lactam compounds (such as compounds II-XIII above) and the lipid modulating agents discussed above, for example, they contain one or more different atoms, have a different arrangement of atoms or a different number of one or more atoms than the sterol absorption inhibitor(s) or lipid modulating agents discussed above. Useful blood modifiers include but are not limited to anti-coagulants (argatroban, bivalirudin, dalteparin sodium, desirudin, dicumarol, lyapolate sodium, nafamostat mesylate, phenprocoumon, tinzaparin sodium, warfarin sodium); antithrombotic (Abcoximab, aspirin, anagrelide hydrochloride, Beraprost, bivalirudin, cilostazol, Carbasalate calcium, Cloricromen, Clopidogrel, dalteparin sodium, danaparoid sodium, dazoxiben hydrochloride, Ditazole, Ditazole, Dipyridamole, Eptifibatide, efegatran sulfate, enoxaparin sodium, fluretofen, ifetroban, ifetroban sodium, Indobufen, Iloprost, lamifiban, lotrafiban hydrochloride, napsagatran, orbofiban acetate, Picotamide, Prasugrel, Prostacyclin, Treprostinil, Ticlopidine, Treprostinil, Triflusal, roxifiban acetate, sibrafiban, tinzaparin sodium, trifenagrel, abciximab, vitamin K antagonists, zolimomab aritox, enzymes such as Alteplase, Ancrod, Anistreplase, Brinase, Drotrecogin alfa, Fibrinolysin, Protein C, Reteplase, Saruplase, Steptokinase, Tenecteplase, and Urokinase), other antithrobotic agents such as Aragatroban, Bivalirudin, Dabigatran, Desirudin, Jirduin, Lepirudin, Melagatran, and Ximelagatran); fibrinogen receptor antagonists (roxifiban acetate, fradafiban, orbofiban, lotrafiban hydrochloride, tirofiban, xemilofiban, monoclonal antibody 7E3, sibrafiban); platelet inhibitors (cilostazol, clopidogrel bisulfate (marketed as Plavix®), epoprostenol, epoprostenol sodium, ticlopidine hydrochloride, aspirin, ibuprofen, naproxen, sulindac, idomethacin, mefenamate, droxicam, diclofenac, sulfinpyrazone, piroxicam, dipyridamole); platelet aggregation inhibitors (acadesine, beraprost, beraprost sodium, ciprostene calcium, itazigrel, lifarizine, lotrafiban hydrochloride, orbofiban acetate, oxagrelate, fradafiban, orbofiban, tirofiban, xemilofiban); hemorrheologic agents (pentoxifylline); lipoprotein associated coagulation inhibitors; Factor VIIa inhibitors (4H-31-benzoxazin-4-ones, 4H-3,1-benzoxazin-4-thiones, quinazolin-4-ones, quinazolin-4-thiones, benzothiazin-4-ones, imidazolyl-boronic acid-derived peptide analogues TFPI-derived peptides, naphthalene-2-sulfonic acid {1-[3-(aminoiminomethyl)-benzyl]-2-oxo-pyrrolidin-3-(S)-yl} amide trifluoroacetate, dibenzofuran-2-sulfonic acid {1-[3-(aminomethyl)-benzyl]-5-oxo-pyrrolidin-3-yl}-amide, tolulene-4-sulfonic acid {1-[3-(aminoiminomethyl)-benzyl]-2-oxo-pyrrolidin-3-(S)-yl}-amide trifluoroacetate, 3,4-dihydro-1H-isoquinoline-2-sulfonic acid {1-[3-(aminoiminomethyl)-benzyl]-2-oxo-pyrrolin-3-(S)-yl}-amide trifluoroacetate); Factor Xa inhibitors (disubstituted pyrazolines, disubstituted triazolines, substituted n-[(aminoiminomethyl)phenyl] propylamides, substituted n-[(aminomethyl)phenyl] propylamides, tissue factor pathway inhibitor (TFPI), low molecular weight heparins (such as dalteparin sodium, marketed as FRAGMIN®), heparinoids, benzimidazolines, benzoxazolinones, benzopiperazinones, indanones, dibasic (amidinoaryl) propanoic acid derivatives, amidinophenyl-pyrrolidines, amidinophenyl-pyrrolines, amidinophenyl-isoxazolidines, amidinoindoles, amidinoazoles, bis-arlysulfonylaminobenzamide derivatives, peptidic Factor Xa inhibitors).

The compositions, therapeutic combinations or methods of the present invention can comprise at least one compound of Formula (I), or pharmaceutically acceptable salts, solvates, isomers or esters thereof, and one or more cardiovascular agents which are chemically different from the substituted azetidinone and substituted β-lactam compounds (such as compounds II-XIII above) and the lipid modulating agents discussed above, for example, they contain one or more different atoms, have a different arrangement of atoms or a different number of one or more atoms than the sterol absorption inhibitor(s) or PPAR receptor activators discussed above. Useful cardiovascular agents include but are not limited to calcium channel blockers (clentiazem maleate, amlodipine besylate (marketed as NORVASC® and LOTREL®), isradipine, nimodipine, felodipine (marketed as PLENDIL®), nilvadipine, nifedipine, teludipine hydrochloride, diltiazem hydrochloride (marketed as CARDIZEM®), belfosdil, verapamil hydrochloride (marketed as CALAN®), fostedil), nifedipine (marketed as ADALAT®), nicardipine (marketed as CARDENE®), nisoldipine (marketed as SULAR®), bepridil (marketed as VASCOR®); adrenergic blockers (fenspiride hydrochloride, labetalol hydrochloride, proroxan, alfuzosin hydrochloride, acebutolol, acebutolol hydrochloride, alprenolol hydrochloride, atenolol, bunolol hydrochloride, carteolol hydrochloride, celiprolol hydrochloride, cetamolol hydrochloride, cicloprolol hydrochloride, dexpropranolol hydrochloride, diacetolol hydrochloride, dilevalol hydrochloride, esmolol hydrochloride, exaprolol hydrochloride, flestolol sulfate, labetalol hydrochloride, levobetaxolol hydrochloride, levobunolol hydrochloride, metalol hydrochloride, metoprolol, metoprolol tartrate, nadolol, pamatolol sulfate, penbutolol sulfate, practolol, propranolol hydrochloride, sotalol hydrochloride, timolol, timolol maleate, tiprenolol hydrochloride, tolamolol, bisoprolol, bisoprolol fumarate, nebivolol); adrenergic stimulants; angiotensin converting enzyme (ACE) inhibitors (benazepril hydrochloride (marketed as LOTENSIN®), benazeprilat, captopril (marketed as CAPTOEN®), delapril hydrochloride, fosinopril sodium, libenzapril, moexipril hydrochloride (marketed as UNIVASC®), pentopril, perindopril, quinapril hydrochloride (marketed as ACCUPRIL®), quinaprilat, ramipril (marketed as RAMACE® and ALTACE®) (or ACE/NEP inhibitors such as ramipril, marketed as DELIX®/TRITACE®), spirapril hydrochloride, peridopril, (marketed as ACEON®), spiraprilat, trandolapil (marketed as MAVIK®), teprotide, enalapril maleate (marketed as VASOTEC®), lisinopril (marketed as ZESTRIL®), zofenopril calcium, perindopril erbumine); antihypertensive agents (althiazide, benzthiazide, captopril, carvedilol, chlorothiazide sodium, clonidine hydrochloride, cyclothiazide, delapril hydrochloride, dilevalol hydrochloride, doxazosin mesylate, fosinopril sodium (marketed as MONOPRIL®), guanfacine hydrochloride, lomerizine, methyldopa, metoprolol succinate, moexipril hydrochloride, monatepil maleate, pelanserin hydrochloride, phenoxybenzamine hydrochloride, prazosin hydrochloride, primidolol, quinapril hydrochloride, quinaprilat, ramipril, terazosin hydrochloride, candesartan, candesartan cilexetil, telmisartan, amlodipine besylate, amlodipine maleate, bevantolol hydrochloride); angiotensin II receptor antagonists (candesartan, irbesartan, losartan potassium, candesartan cilexetil, telmisartan); anti-anginal agents (amlodipine besylate, amlodipine maleate, betaxolol hydrochloride, bevantolol hydrochloride, butoprozine hydrochloride, carvedilol, cinepazet maleate, metoprolol succinate, molsidomine, monatepil maleate, primidolol, ranolazine hydrochloride, tosifen, verapamil hydrochloride); coronary vasodilators (fostedil, azaclorzine hydrochloride, chromonar hydrochloride, clonitrate, diltiazem hydrochloride, dipyridamole, droprenilamine, erythrityl tetranitrate, isosorbide dinitrate, isosorbide mononitrate, lidoflazine, mioflazine hydrochloride, mixidine, molsidomine, nicorandil, nifedipine, nisoldipine, nitroglycerine, oxprenolol hydrochloride, pentrinitrol, perhexyline maleate, prenylamine, propatyl nitrate, terodiline hydrochloride, tolamolol, verapamil); diuretics (the combination product of hydrochlorothiazide and spironolactone and the combination product of hydrochlorothiazide and triamterene).

The compositions, therapeutic combinations or methods of the present invention can comprise at least one compound of Formula (I), or pharmaceutically acceptable salts, solvates, isomers or esters thereof, and one or more antidiabetic medications for reducing blood glucose levels in a patient. Useful antidiabetic medications include, but are not limited to, drugs that reduce energy intake or suppress appetite, drugs that increase energy expenditure and nutrient-partitioning agents. Suitable antidiabetic medications include, but are not limited to, sulfonylurea (such as acetohexamide, chlorpropamide, gliamilide, gliclazide, glimepiride, glipizide, glyburide, glibenclamide, tolazamide, and tolbutamide), meglitinide (such as repaglinide and nateglinide), biguanide (such as metformin and buformin), alpha-glucosidase inhibitor (such as acarbose, miglitol, camiglibose, and voglibose), certain peptides (such as amlintide, pramlintide, exendin, and GLP-1 agonistic peptides), and orally administrable insulin or insulin composition for intestinal delivery thereof. Generally, a total dosage of the above-described antidiabetic medications can range from 0.1 to 1,000 mg/day in single or 2-4 divided doses.

Mixtures of two, three, four or more of any of the pharmacological or therapeutic agents described above can be used in the compositions and therapeutic combinations of the present invention.

Since the present invention relates to treating conditions as discussed above, by treatment with a combination of active ingredients wherein the active ingredients may be administered separately, the invention also relates to combining separate pharmaceutical compositions in kit form. That is, a kit is contemplated wherein two separate units are combined: a pharmaceutical composition comprising at least one selective CB₁ receptor antagonist of Formula (I), or a pharmaceutically acceptable salt, solvate, isomer, or ester thereof, and a separate pharmaceutical composition comprising at least one cholesterol lowering compound as described above. The kit will preferably include directions for the administration of the separate components. The kit form is particularly advantageous when the separate components must be administered in different dosage forms (e.g., oral and parenteral) or are administered at different dosage intervals.

In yet another embodiment, the present invention provides a method of treating, reducing, or ameliorating a disease or condition selected from the group consisting of metabolic syndrome, obesity, waist circumference, abdominal girth, lipid profile, insulin sensitivity, neuroinflammatory disorders, cognitive disorders, psychosis, addictive behavior, gastrointestinal disorders, vascular conditions, hyperlipidaemia, atherosclerosis, hypercholesterolemia, sitosterolemia, vascular inflammation, stroke, diabetes, and cardiovascular conditions, and/or reduce the level of sterol(s) in a patient in need thereof, comprising administering to said patient an effective amount of at least one compound of Formula (I), or a pharmaceutically acceptable salt, solvate, isomer, or ester thereof, and one or more cholesterol lowering compound.

The treatment compositions and therapeutic combinations comprising at least one compound of Formula (I) and at least one cholesterol lowering agent can inhibit the intestinal absorption of cholesterol in mammals can be useful in the treatment and/or prevention of conditions, for example vascular conditions, such as atherosclerosis, hypercholesterolemia and sitosterolemia, stroke, obesity and lowering of plasma levels of cholesterol in mammals, in particular in mammals.

In another embodiment of the present invention, the compositions and therapeutic combinations of the present invention can inhibit sterol or 5α-stanol absorption or reduce plasma concentration of at least one sterol selected from the group consisting of phytosterols (such as sitosterol, campesterol, stigmasterol and avenosterol) and/or 5α-stanol (such as cholestanol, 5α-campestanol, 5α-sitostanol), cholesterol and mixtures thereof. The plasma concentration can be reduced by administering to a mammal in need of such treatment an effective amount of at least one treatment composition or therapeutic combination comprising at least one selective CB₁ receptor antagonist and at least one cholesterol lowering compound, for example a sterol absorption inhibitor described above. The reduction in plasma concentration of sterols or 5α-stanols can range from about 1 to about 70 percent, and preferably about 10 to about 50 percent. Methods of measuring serum total blood cholesterol and total LDL cholesterol are well known to those skilled in the art and for example include those disclosed in PCT WO 99/38498 at page 11, incorporated by reference herein. Methods of determining levels of other sterols in serum are disclosed in H. Gylling et al., “Serum Sterols During Stanol Ester Feeding in a Mildly Hypercholesterolemic Population”, J. Lipid Res. 40: 593-600 (1999), incorporated by reference herein.

The treatments of the present invention can also reduce the size or presence of plaque deposits in vascular vessels. The plaque volume can be measured using (IVUS), in which a tiny ultrasound probe is inserted into an artery to directly image and measure the size of atherosclerotic plaques, in a manner well known to those skilled in the art.

### Synthesis

The following may be referred to herein by the abbreviations indicated: tetrahydropyran (THP), tetrahydrofuran (THF), methanesulfonyl chloride (MsCl), 1-chloroethyl chloroformate (ACECI), ethyl acetate (EtOAc), 1,1′-bis(diphenylphosphino)ferrocene (dppf), ethanol (EtOH), N-(3-dimethylaminopropyl)-N′-ethylcarbodiimide hydrochloride (EDC), 1-[3-(dimethylamino)propyl]-3-ethylcarbodiimide methiodide (EDCI), 1-hydroxybenzotriazole (HOBT), N,N-dimethylformamide (DMF), acetonitirile (MeCN), propionitrile (EtCN), N-methylmorpholine-N-oxide (NMO), 3-chloroperoxybenzoic acid (MCPBA), methanol (MeOH), room temperature (RT), liquid chromatography mass spectrometry (LCMS), high performance liquid chromatography (HPLC), and thin-layer chromatography (TLC).

Piperazines g are prepared according the steps outlined in Scheme A. A benzyl protected ethanolamine a can be heated with an epoxide b to furnish a mixture of the amino-alcohols c and d. The alcohols c and d can be converted into the diamine e via sequential treatment with MsCl followed by Are NH₂. The diamine e can be converted into the piperazine f via deprotection of the THP group in e followed by activation of the alcohol. The benzyl group in f can be removed via treatment with ACECI followed by basic hydrolysis which provides piperazines g.

All LCMS (MH⁺) values reported herein are the observed values.

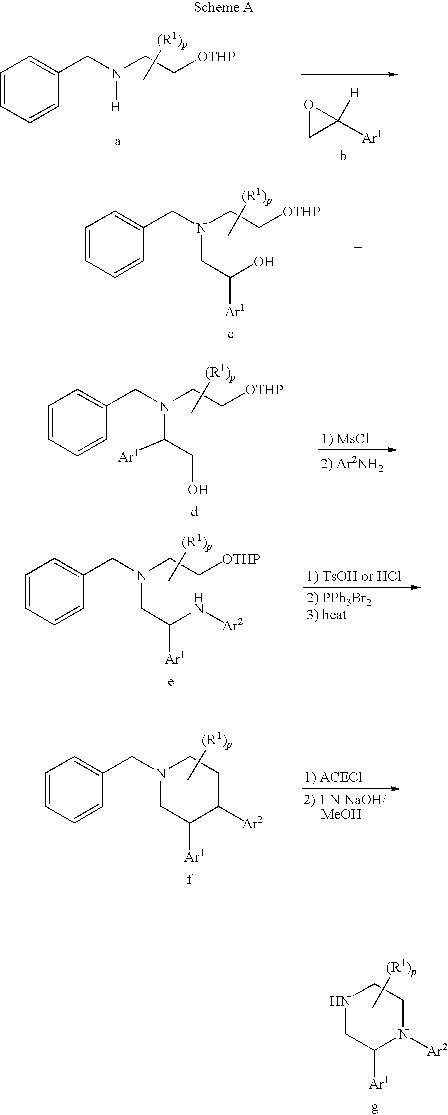

Also, chiral epoxides, such as h and i, can be utilized as that described in Scheme A to provide enantiopure piperazines j and k (Scheme B). The chiral epoxides can be prepared either via asymmetric di-hydroxylation of a styrene (e.g. Sharpless AD mix a or p) or chiral reduction of a bromo-ketone (e.g. CBS reduction). These methods allow the preparation of either enantiomer of the epoxide, h or i.

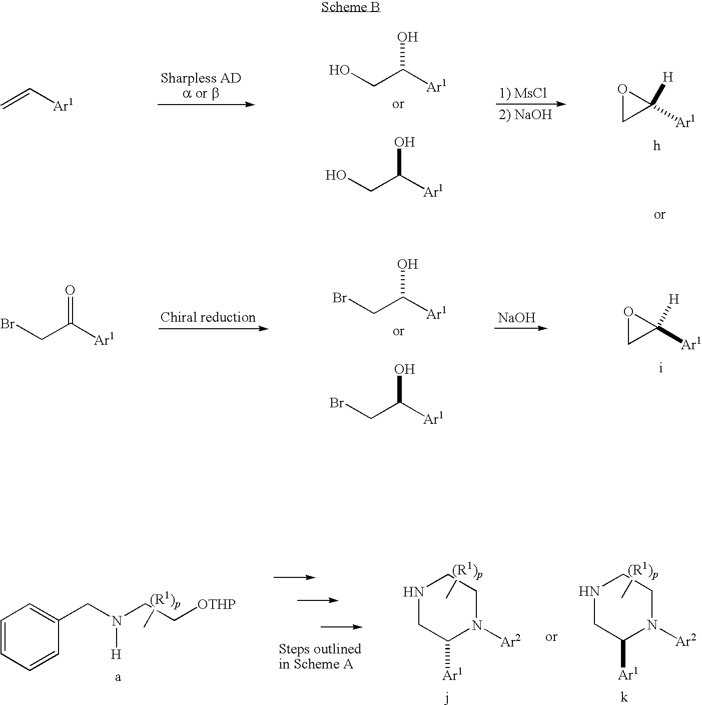

Further functionalization of piperazine g into compounds is illustrated in Scheme C. piperazine g can be transformed into the alkylated derivatives such as l and m via reductive alkylation (Na(AcO)₃BH/XC(O)R²) and/or direct alkylation (base/X(R²)₂₀ Ms) conditions. Also, the piperazine g can be converted into an amide or sulfonamide using standard techniques (e.g. n and o). Hydroxy-ethyl analogs p can be made via reaction of a hydroxy-mesylate or epoxide with piperazine g.

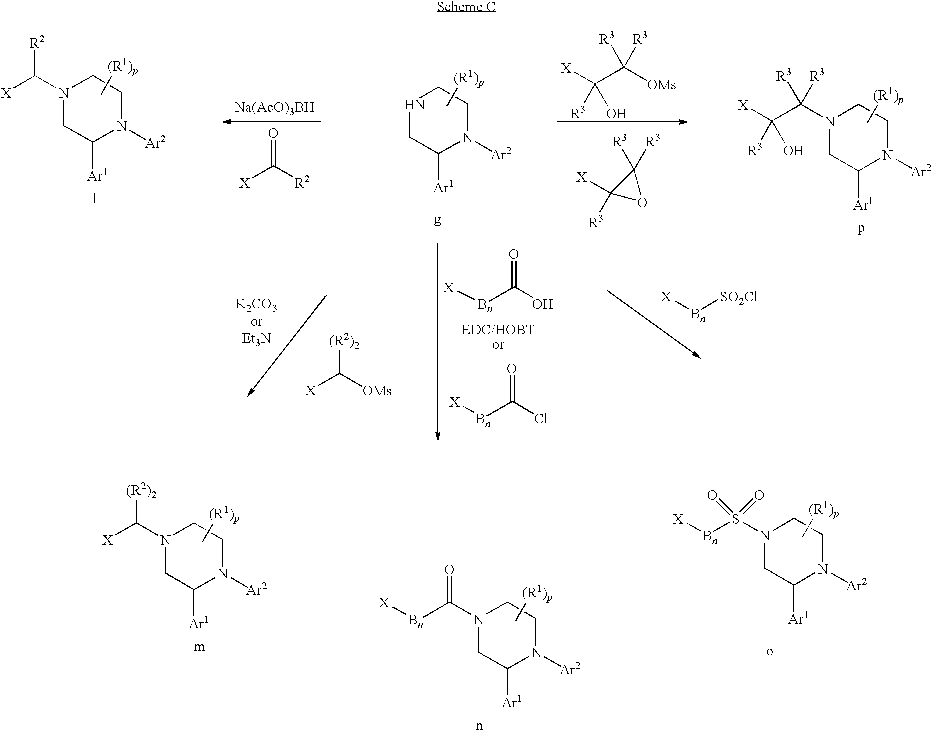

Also, the chiral piperazine j can be functionalized according to the transformations outlined in Scheme C to furnish the corresponding chiral derivatives (Scheme D).

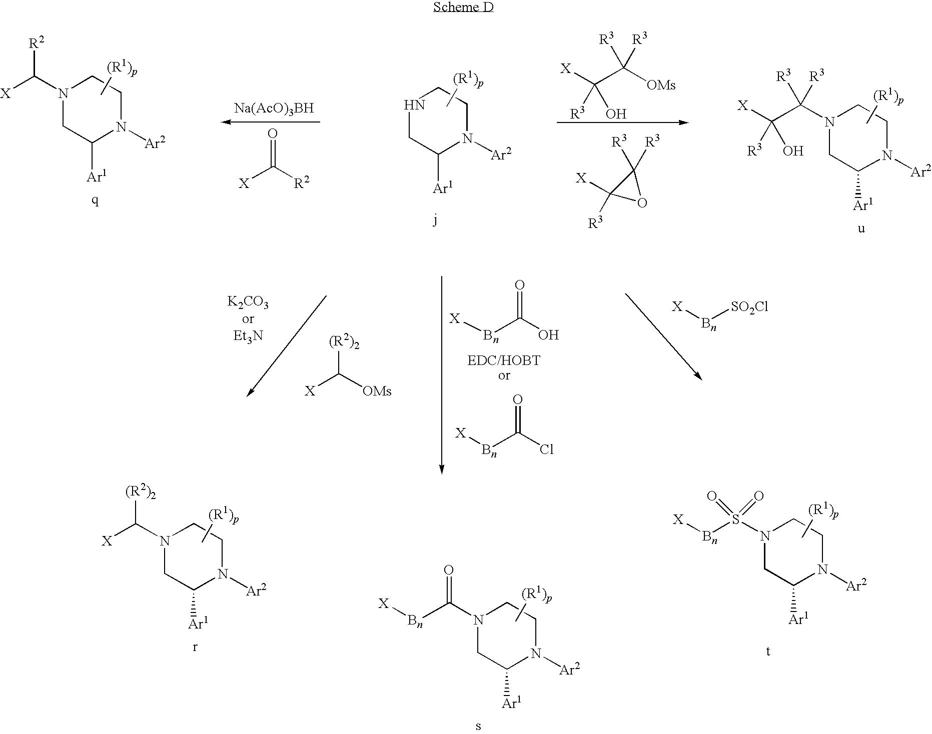

Also, the chiral piperazine k can be functionalized according to the transformations outlined in Scheme C to furnish the corresponding chiral derivatives (Scheme E).

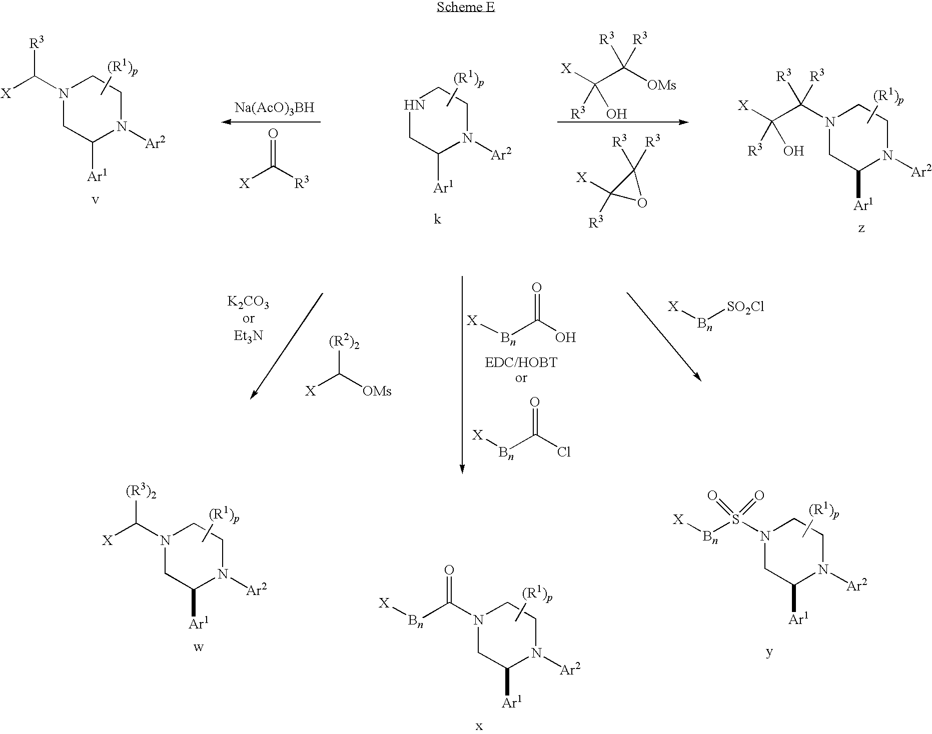

Certain reagents for functionalization of the piperazine core can be prepared in chiral form. These reagents can be prepared by known procedures in the art, and non-limiting examples are illustrated below.

A ketone can be transformed into either enantiomer of the corresponding alcohol by several methods (1.reduction 2. enzymatic resolution or chiral reduction). Activation of the alcohol (MsCl/Et₃N) provides the either enantiomer of the mesylate which can be coupled to either enantiomer of the piperazine (j or k) which provides access to four possible diastereomers in pure form (e.g. aa, ab, ac, or ad; Scheme F).

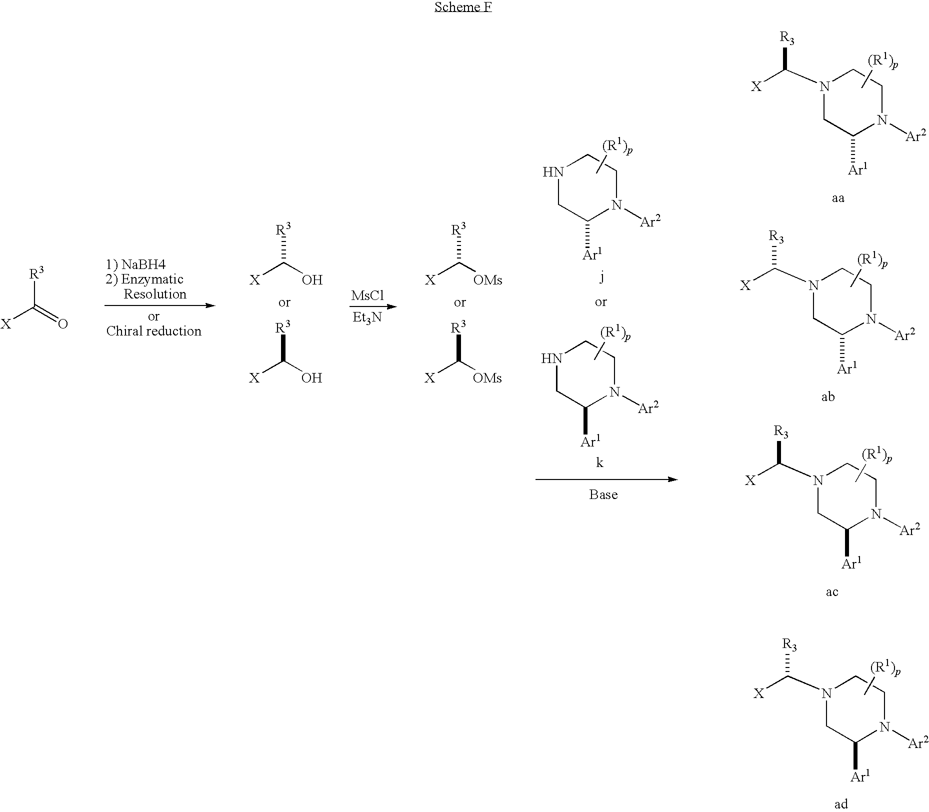

Using procedures known in the art, substituted alkenes can be prepared from olefination of ketones (Wittig) and/or transition metal mediated methods (Pd(0)/metal-alkenyl derivative). These can be transformed into chiral diols via asymmetric methods (e.g. Sharpless AD mix a or 13). The formed chiral diol can be transformed into the corresponding mesylate and/or epoxide. These can be reacted with the chiral piperazines, j and k, to provide four possible diastereomers in pure form (e.g. ae,af,ag, and ah; Scheme G).

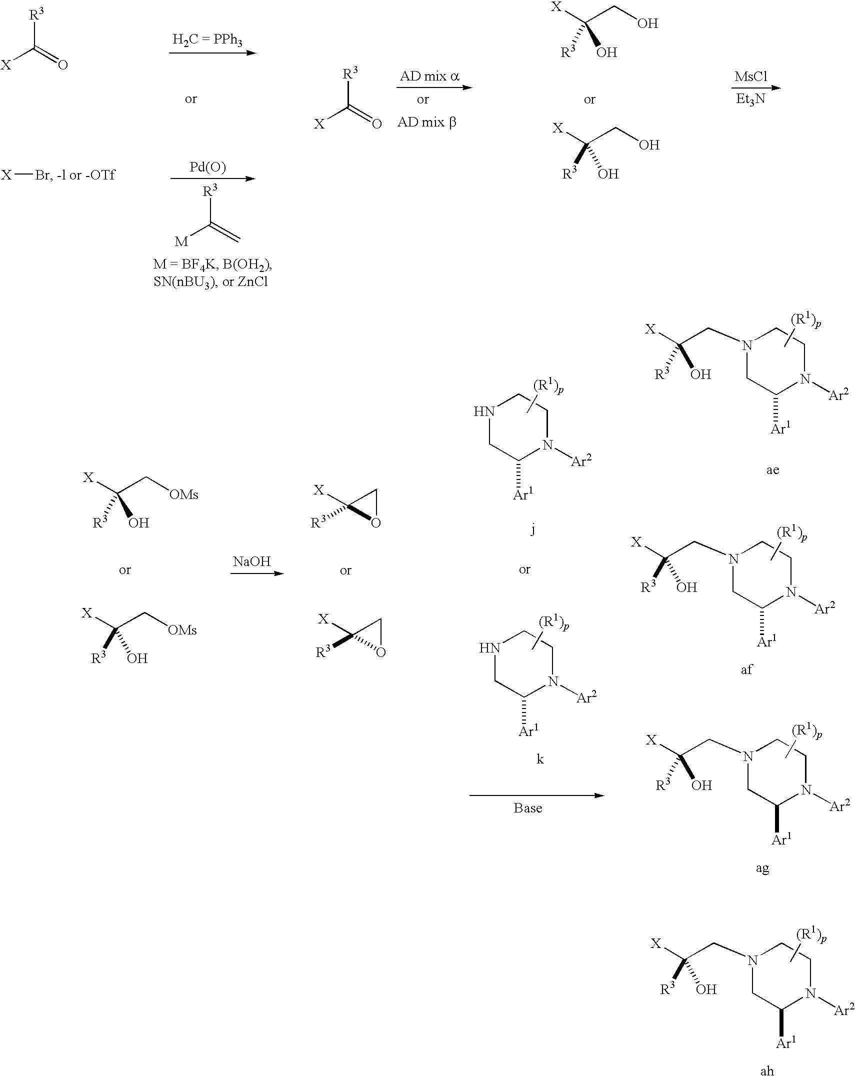

Also, the chiral piperazine cores, j and k, can be reacted with chiral epoxides to produce chiral piperazine-alcohol derivatives ai, aj, ak, and al (Scheme H). The requisite chiral epoxides can be prepared by procedures known in the art (e.g. chiral reduction of a bromo-ketone and/or asymmetric epoxidation of an alkene).

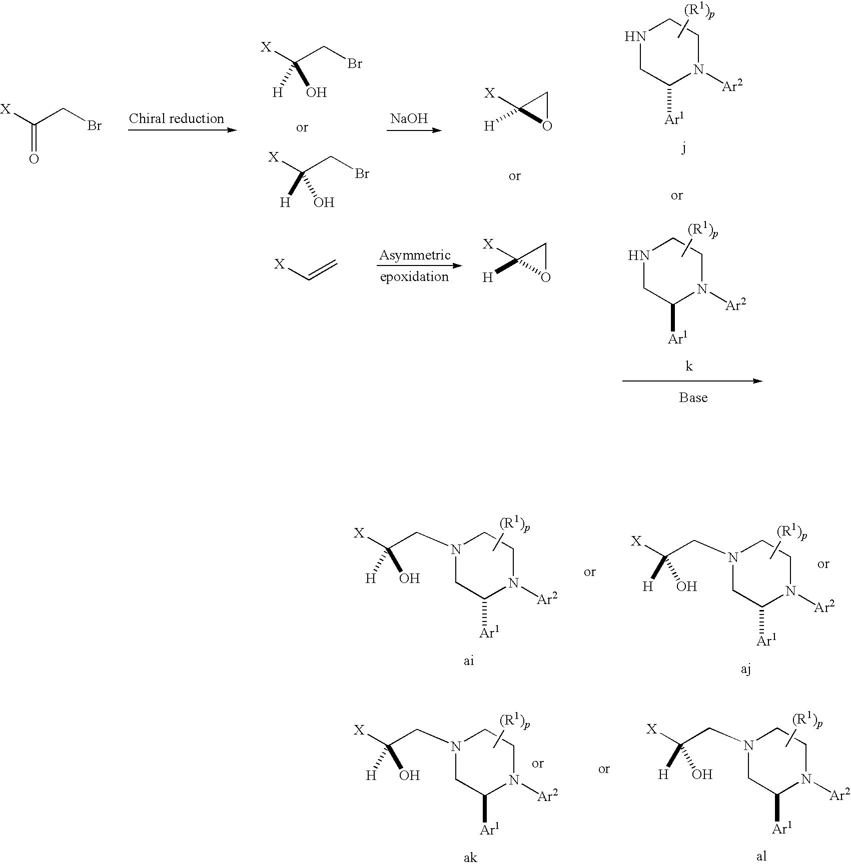

The following examples were prepared according to procedures known in the art.

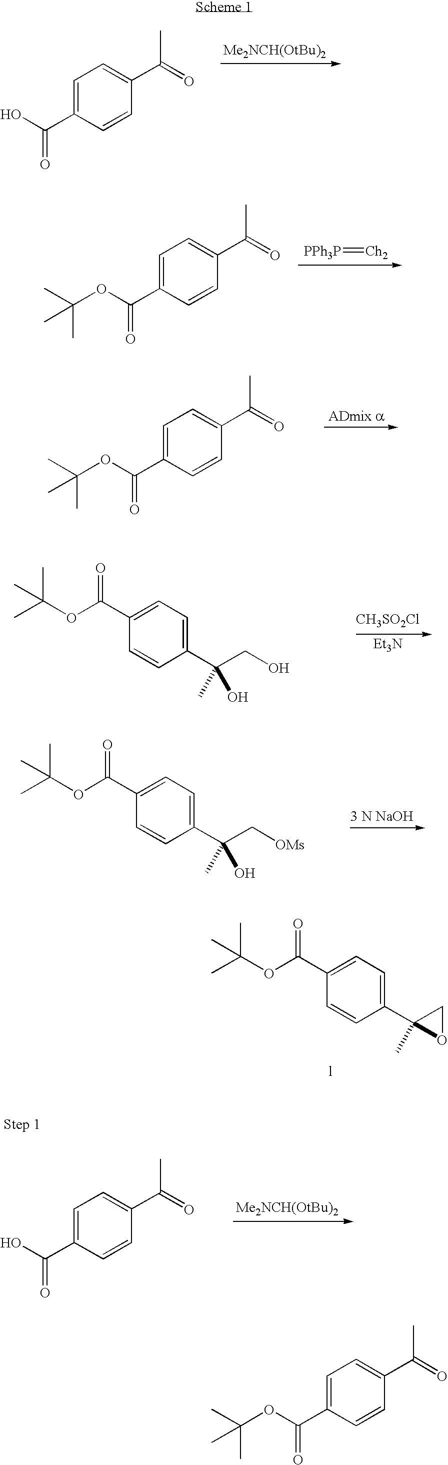

The carboxylic acid (10 g, 60.9 mmol) and Me₂NCH(OtBu)₂ (25 g) were heated in toluene (300 mL) for 5 hours (85° C.). More Me₂NCH(OtBu)₂ (25 g) was added, and the reaction was heated at 85° C. for 14 hours. The solution was partitioned between EtOAc and sat. NaHCO_{3(aq.)}. The aqueous layer was extracted with EtOAc. The combined organic layers were washed with brine and dried over MgSO₄. The solution residue was filtered through a plug of SiO₂ rinsing with CH₂Cl₂. This afforded 4.7 g (35%) of the tert-butyl ester as a solid.

### Step 2

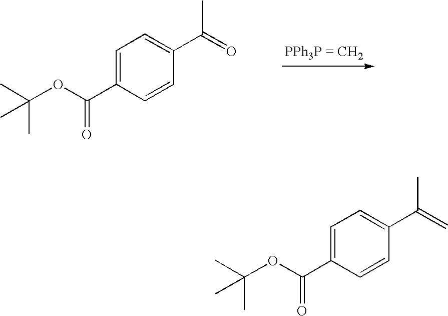

Methyl triphenylphosphonium bromide (15.3 g) was suspended in THF (100 mL) at 0° C. n-Butyllithium (25.6 mL of a 1.6 M solution in hexanes) was added dropwise at 0° C. The yellow solution was stirred at 0° C. (1 h). The ketone (4.7 g, 21.4 mmol) was added, and the resulting slurry was stirred at 25° C. (18 h). The mixture was quenched with water, and the mixture was extracted with Et₂O. The combined Et₂O layers were washed with brine and dried (MgSO₄). The mixture was filtered and concentrated. The residue was filtered through a plug of SiO₂ (rinsing with CH₂Cl₂). The solution was concentrated. The residue was purified via gradient flash chromatography (5/1 hexanes/EtOAc, SiO₂) which furnished 2.45 g (52%) of the alkene as a colorless oil.

### Step 3

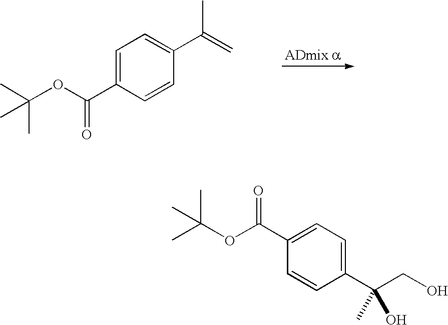

The alkene (2.45 g, 11.2 mmol) and AD mix a (17 g) were taken up in tert-butanol/water (1/1, 90 mL), and the mixture was stirred at 25° C. (3 days). The mixture was cooled to 0° C., and water (150 mL) was added. Solid Na₂SO₃ (17 g) was added slowly to the mixture at 0° C. The solution was stirred at 0° C. (1 h) and then at 25° C. (1 h). The mixture was extracted with EtOAc. The combined organic layers were washed with brine and dried (MgSO₄). The solution was filtered and concentrated to give the crude diol. The residue was purified via gradient flash chromatography (1/1 hexanes/EtOAc, SiO₂) which furnished 1.97 g (70%) of the alkene as a colorless oil that slowly solidified.

### Step 4

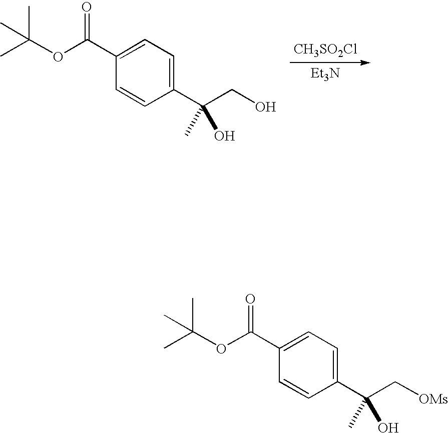

The diol (1.97 g, 7.8 mmol) and Et₃N (1.3 mL) were taken up in CH₂Cl₂ at 0° C. Methanesulfonyl chloride (1.2 mL) in CH₂Cl₂ (20 mL) was added dropwise at 0° C. The solution was stirred at 0° C. for 15 minutes. The solution was washed with sat. NaHCO_{3(aq.)}. The aqueous layer was extracted with CH₂Cl₂. The combined organic layers were dried (MgSO₄), filtered, and concentrated. The mesylate was used in the next reaction without further purification.

### Step 5

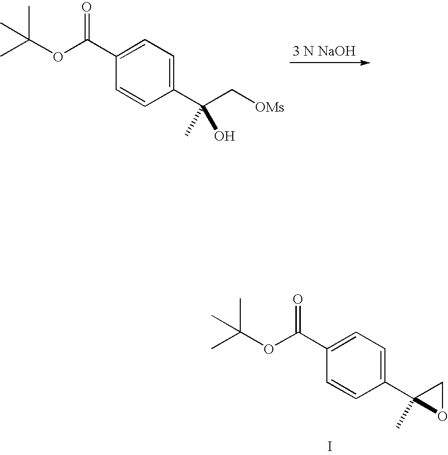

The mesylate from Step 4 was partitioned between toluene and 3 N NaOH_{(aq.)} (1/1, 80 ml), and the resulting mixture was stirred at 25° C. for 2 h. The layers were separated, and the aqueous layer was extracted with EtOAc. The combined organic layers were washed with brine and dried (MgSO₄). Filtration and concentration provided a yellow oil. Purification of the residue via gradient flash chromatography (0-10% EtOAc/hexanes, SiO₂) gave the epoxide I as a colorless oil.

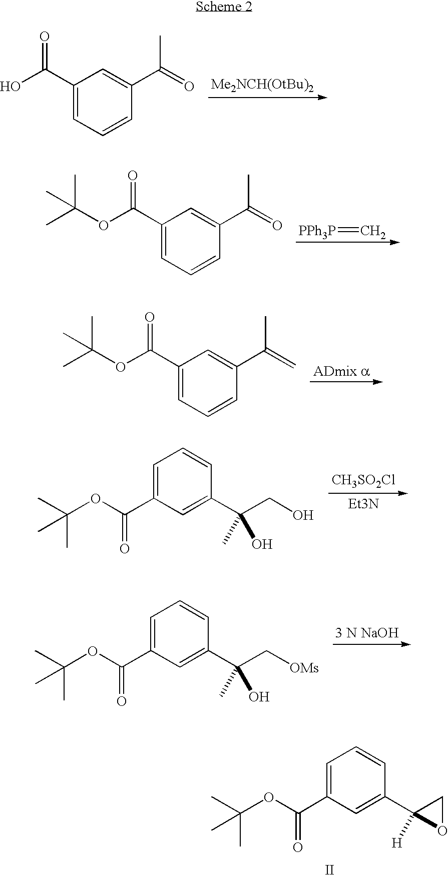

In an analogous fashion, the 1,3-disubstituted ketone was processed as that described above for the 1,4-disubstituted analog (Scheme 1) which provided the 1,3-disubstituted ester-epoxide II (Scheme 2).

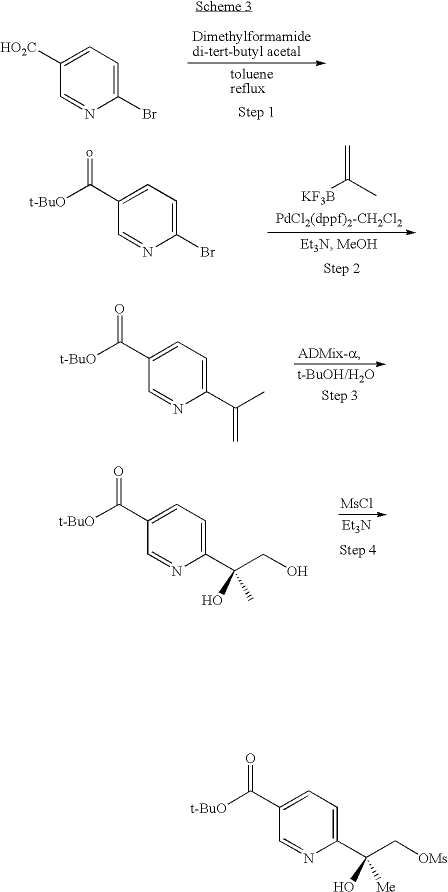

### Step 1:

To a solution of the 6-bromonicotinic acid (2.5 g, 12.4 mmol) in toluene (25 mL) was added dimethylformamide di-tert-butylacetal (5.0 g, 24.8 mmol). The solution was then heated to relfux overnight. Additional dimethylformamide di-tert-butylacetal (10.0 g, 59.6 mmol) was added in two portions over 24 h with continued stirring at reflux. The solution was stirred at reflux for a total of 72 h then cooled to RT. To the solution was added sat. NaHCO₃ (aq.) and the aqueous layer was extracted with EtOAc (3×). The combined organic layers were washed with brine, dried over Na₂SO₄, filtered and concentrated. The crude product was purified via flash chromatography (SiO₂: gradient elution, 100:0 to 92:8 hexanes:EtOAc) to afford the ester (1.68 g, 52%).

### Step 2:

To a solution of the bromide from step 1 (1.68 g, 6.5 mmol) in MeOH (20 mL) in a pressure tube was added potassium trifluoro(prop-1-en-2-yl)borate (J. Am. Chem. Soc 2003, 125, 11148-11149) (1.16 g, 7.8 mmol). The resultant slurry was degassed by bubbling N₂ through the solvent for 10 min. To this slurry was then added PdCl₂(dppf)₂.CH₂Cl₂ (159 mg, 0.20 mmol) and Et₃N (657 mg, 6.5 mmol). The pressure tube was sealed and the mixture was heated to 100° C. with stirring for 3 h. The mixture was then cooled to RT, transferred to a round bottom flask and concentrated. The crude product was partitioned between water and EtOAc. The aqueous layer was extracted with EtOAc (3×). The combined organic layers were dried over Na₂SO₄, filtered and concentrated. The crude product was purified via flash chromatography (SiO₂: gradient elution, 100:0 to 92:8 hexanes:EtOAc) to afford the styrene (1.24 g, 86%) as a clear oil.

### Step 3:

To a biphasic mixture of the styrene from Step 2 (1.24 g, 5.7 mmol) in 1:1 tert-butanol/water (60 mL) was added AD mix a (Aldrich) (7.9 g) and methane sulfonamide (492 mg, 5.2 mmol). The resultant mixture was stirred vigorously at RT for 48 h. After that time, Na₂SO₃ (8.0 g) was added and the mixture was stirred at RT for 1 h. The mixture was then diluted with 2-propanol and stirred for 1 h. The organic layer was separated, dried over Na₂SO₄, filtered and concentrated. The crude product was purified via flash chromatography (SiO₂: gradient elution, 100:0 to 50:50 hexanes:EtOAc) to afford the diol (1.18 g, 82%) as a clear oil that crystallized upon standing.

### Step 4:

To a solution of the diol (1.37 g, 5.4 mmol) in CH₂Cl₂ (30 mL) was added Et₃N (650 mg, 6.5 mmol) followed by methanesulfonyl chloride (744 mg, 6.5 mmol). The solution was stirred at RT for 3 h. After that time, the solution was diluted with CH₂Cl₂ and washed with NaHCO₃ (aq.). The aqueous layer was extracted with CH₂Cl₂ (2×). The combined organic layers were dried over Na₂SO₄, filtered and concentrated in vacuo. The crude product was purified via flash chromatography (SiO₂: gradient elution, 100:0 to 50:50 hexanes:EtOAc) to afford the mesylate (1.45 g, 81%) as a clear oil.

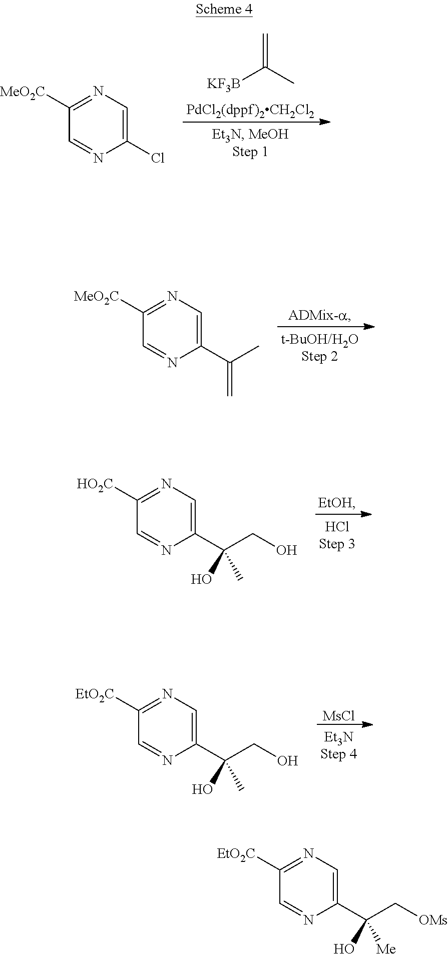

### Step 1:

The styrene was prepared from methyl 5-chloropyrazine-2-carboxylate (Lonza Inc, Allendale, N.J.) using a procedure similar to that described in Scheme 3 Step 2.

### Step 2:

The diol carboxylic acid was prepared using a procedure similar to that described in Scheme 3 Step 3 except the diol from step 1 of this scheme was used.

### Step 3:

To a solution of the carboxylic acid from step 2 (ca 6.5 mmol) in anhydrous EtOH (30 mL) was added a solution of hydrogen chloride (4 M in dioxane, 5 mL). The solution was heated to reflux with stirring for 4 h. After that time, the solution was allowed to cool to RT. The solution was then concentrated in vacuo and used without further purification.

### Step 4:

To a solution was the diol from Step 3 (ca 6.5 mmol) in CH₂Cl₂ (20 mL) was added Et₃N (1.4 g, 14.3 mmol) followed by methane sulfonamide (825 mg, 7.2 mmol). The solution was stirred at RT. After the reaction was complete, the solution was diluted with CH₂Cl₂ and washed with NaHCO₃ (aq.). The organic layer was dried over Na₂SO₄, filtered and concentrated. The crude product was purified via flash chromatography (SiO₂: gradient elution, 100:0 to 50:50 hexanes:EtOAc) to afford the mesylate (600 mg).

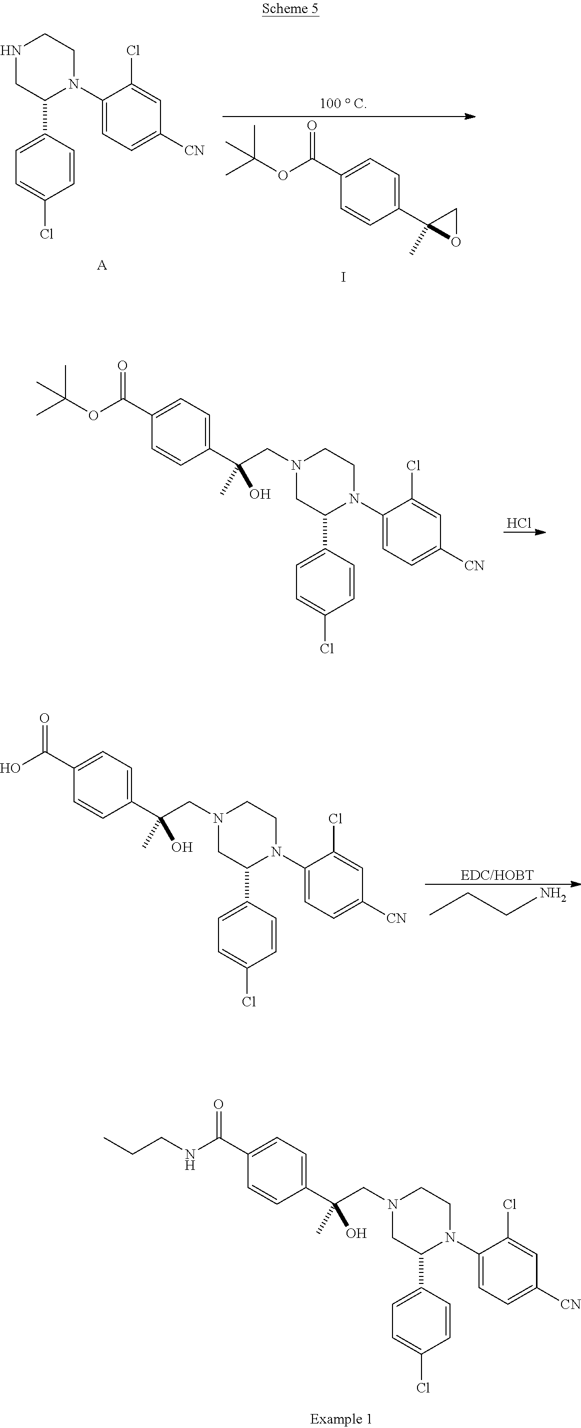

### Step 1

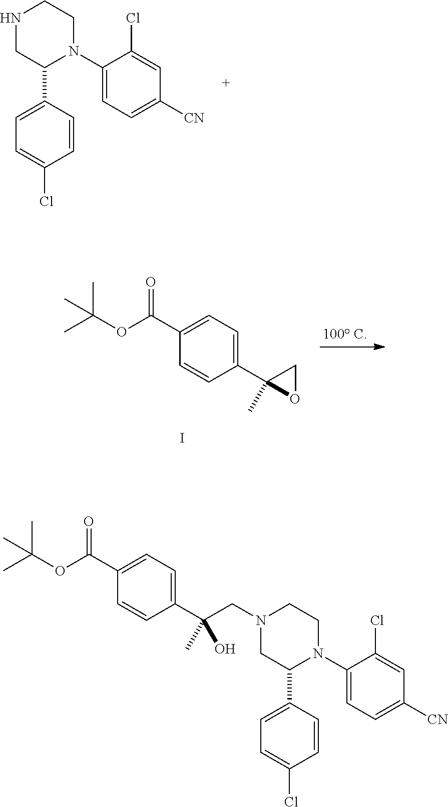

The piperazine A (for preparation see: WO2006060461) (664 mg, 2 mmol) and epoxide I (464 mg, 2 mmol) were heated neat at 100° C. for 18 h. The residue was purified via gradient flash chromatography (0-5% EtOAc/CH₂Cl₂, SiO₂) which gave 570 mg (50%) of the alcohol as a yellow oil.

### Step 2

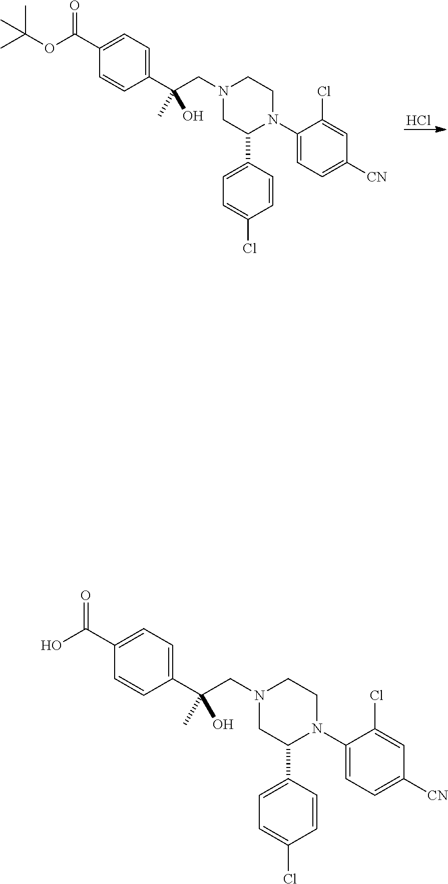

The tert-butyl ester (570 mg, 1 mmol) was taken up in dioxane (2 mL) and 4 M HCl dioxane (5 mL), and the resulting solution was stirred at 25° C. for 18 h. The solution was concentrated which gave ˜580 mg (Quant.) of the piperazine-acid HCl salt as a glass. This material was used without further purification.

### Step 3

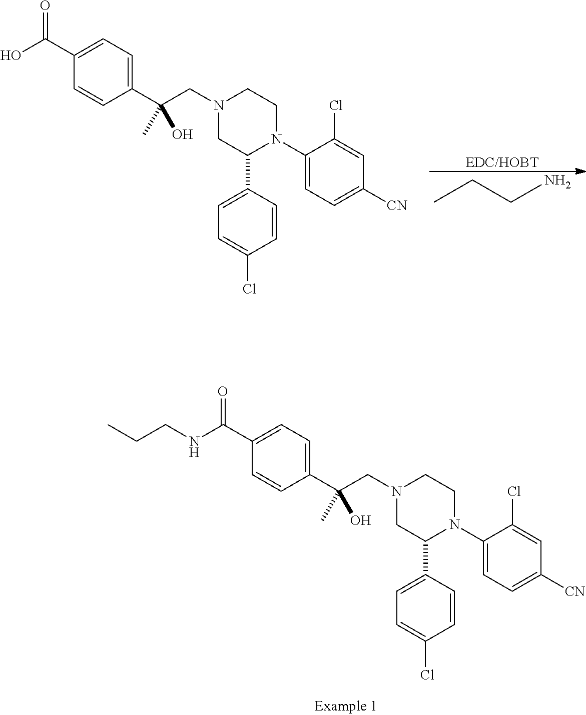

The piperazine acid (100 mg, 0.18 mmol), EDC (70 mg), HOBT (50 mg), and n-propyl amine (0.2 mL) were taken up in (3 mL) and stirred at 25° C. for 18 h. The solution was evaporated. The residue was purified via thin-layer preparative chromatography (4/1 CH₂Cl₂/acetone, SiO₂) which gave 45 mg (45%) of Example 1 as a colorless oil: LCMS (MH⁺) 551.3.

The epoxide I or II, piperazine A or B (for preparation see: WO2006060461), and amine provided additional examples according to the analogous procedures outlined for Example 1 (Table 1).

TABLE 1
(Procedure outlined in Scheme 5)

| | | | | | LCMS (MH⁺) |
|---|---|---|---|---|---|
| epoxide | piperazine | amine | Ex. | Structure | (Observed) |
| I | 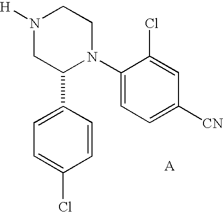 | 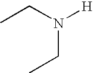 | 2 | 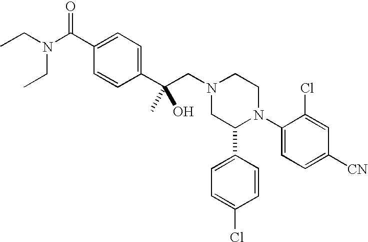 | 565.3 |
| I | 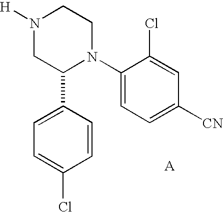 | 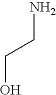 | 3 | 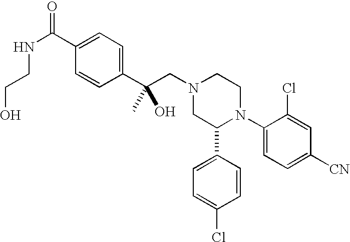 | 553.3 |
| I | 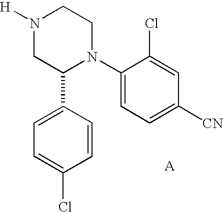 | 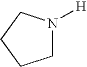 | 4 | 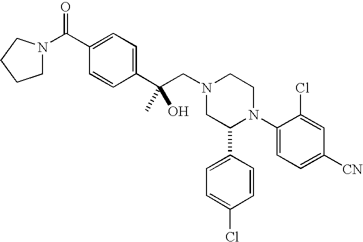 | 563.3 |
| II | 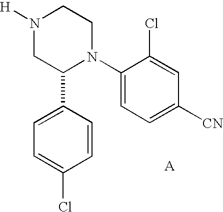 | 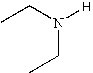 | 5 | 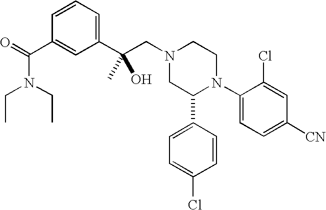 | 565.3 |
| II | 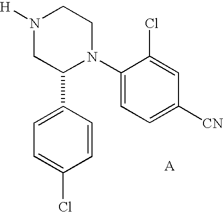 | 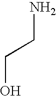 | 6 | 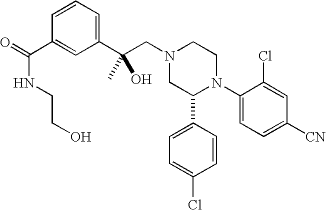 | 553.3 |
| II | 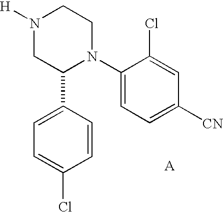 | 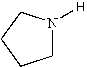 | 7 | 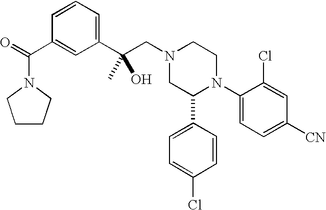 | 563.3 |
| II | 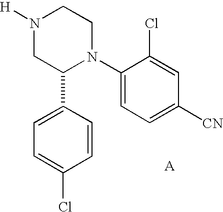 | 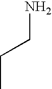 | 8 | 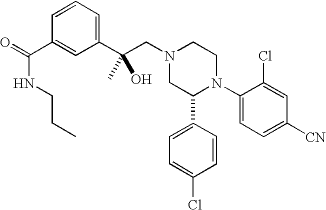 | 551.3 |
| I | 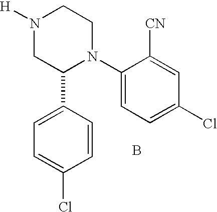 | 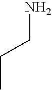 | 9 | 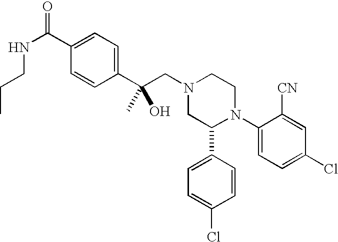 | 551.3 |
| I | 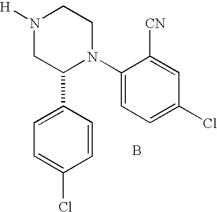 | 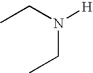 | 10 | 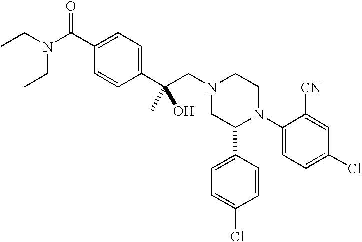 | 565.3 |
| I | 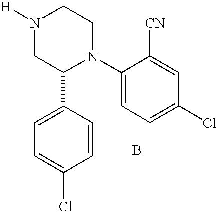 | 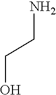 | 11 | 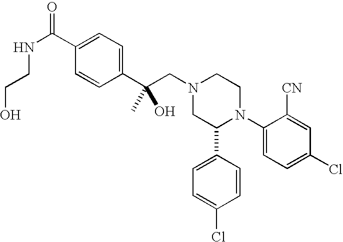 | 553.3 |
| II | 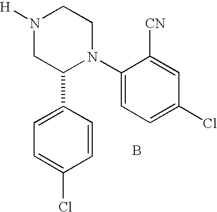 | 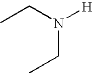 | 12 | 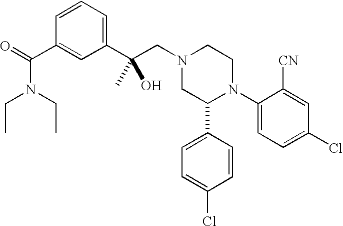 | 565.3 |
| II | 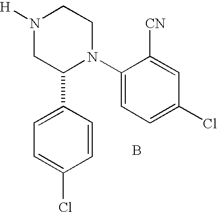 | 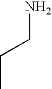 | 13 | 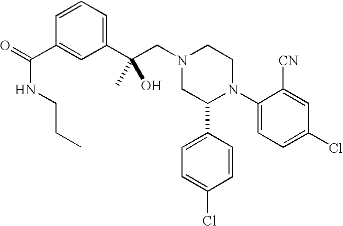 | 551.3 |

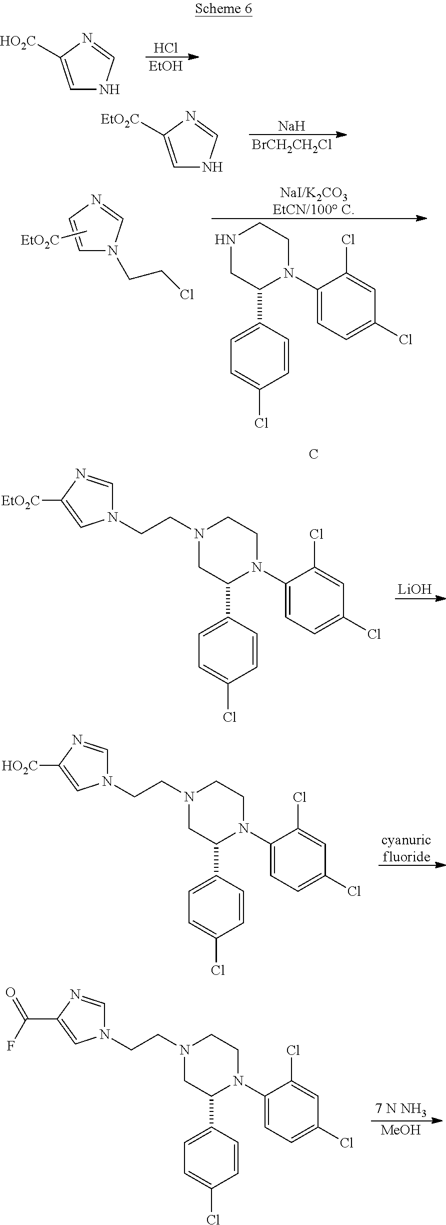

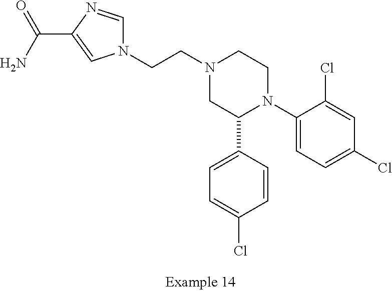

### Step 1

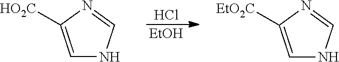

1H-Imidazole-4-carboxylic acid (3.8 g, 33.8 mmol) and 4 M HCl in dioxane (20 mL) were taken up in EtOH (100 mL) and heated at 80° C. for 18 h. The solution was concentrated. The residue was partitioned between EtOAc and water. The mixture was quenched with solid NaHCO₃ until the aqueous layer was no longer acidic. The layers were separated. The aqueous layer was, extracted with EtOAc. The combined organic layers were washed with brine and dried (MgSO₄). Filtration and concentration of the solution provided 2.47 g (52%) of the ethyl ester as a white solid.

### Step 2

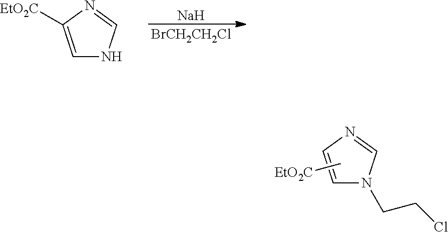

The imidazole-ethyl ester (500 mg, 3.6 mmol) was suspended in DMF (5 mL) at 25° C. Sodium hydride (170 mg, 60 wt % dispersion in oil) was added at 25° C., and the resulting mixture was stirred at 25° C. for 0.5 h. Bromo-chloro ethane (0.7 mL, 8.9 mmol) was added, and the solution was stirred at 25° C. for 16h. The solution was partitioned between EtOAc and water. The aqueous layer was extracted with EtOAc. The combined organic layers were washed with brine and dried (MgSO₄). Filtration and concentration gave a yellow oil. The residue was purified via gradient flash chromatography (0-5% EtOH/CH₂Cl₂, SiO₂) which furnished 790 mg (Quant.) of the chloro-ethyl imidazole as a mixture of regio isomers.

### Step 3

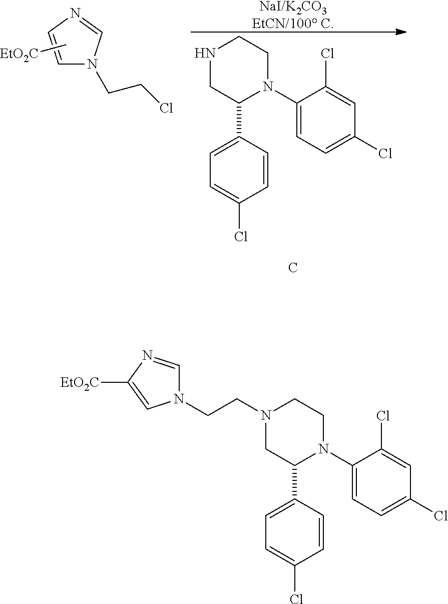

The chloro-ethyl imidazole (3.6 mmol), piperazine C (for preparation see: WO2006060461) (500 mg, 1.46 mmol), NaI (220 mg, 1.46 mmol), and K₂CO₃ (604 mg, 4.4 mmol) were taken up in EtCN (3 mL) and heated at 100° C. for 50 h. The solution was partitioned between EtOAc and water. The aqueous layer was extracted with EtOAc. The combined organic layers were washed with brine and dried (MgSO₄). Filtration and concentration gave a brown oil. The residue was purified via gradient flash chromatography (0-5% EtOAc/CH₂Cl₂, SiO₂). Further purification via thin-layer preparative chromatography (3/1 acetone/CH₂Cl₂, SiO₂) gave 380 mg (51%) of the imidazole-piperazine as a colorless oil.

### Step 4

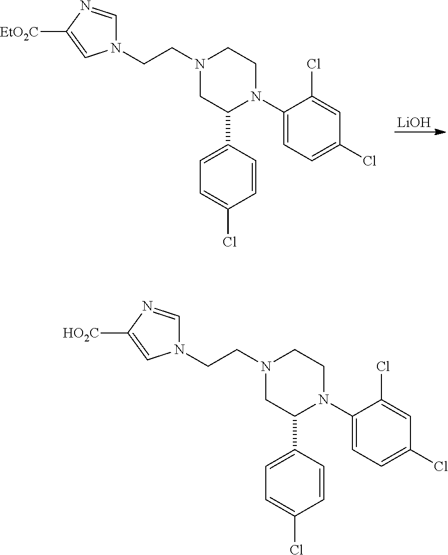

The ethyl ester (339 mg, 0.67 mmol) and LiOH—H₂O (140 mg, 3.3 mmol) were taken up in MeOH/H₂O (1/1, 15 mL) and stirred at 25° C. for 18 h. More LiOH—H₂O (3 g) was added, and the solution was stirred an additional 18 h. The solution was concentrated. The residue was taken up in water and neutralized with 1 M HCl_{(aq.)}. The solution was extracted with CH₂Cl₂. The combined organic layers were dried (MgSO₄). The solution was filtered and concentrated which gave 259 mg (81%) of the acid as a white solid.

### Step 5

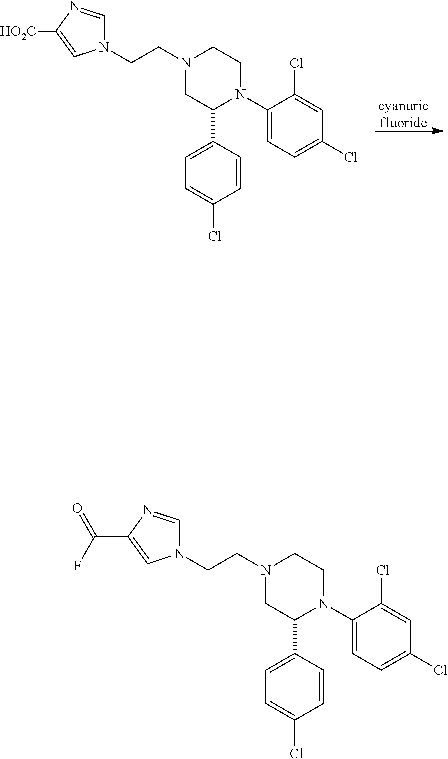

The acid (259 mg, 0.54 mmol) and pyridine (0.15 mL) were taken up in CH₂Cl₂ and cooled to 0° C. Cyanuric fluoride (0.15 mL) was added at 0° C., and the resulting mixture was stirred at 0° C. for 4.5 h. The solution was diluted with CH₂Cl₂ and washed with sat. NaHCO_{3(aq.)}. The aqueous layer was extracted with CH₂Cl₂. The combined organic layers were dried (MgSO₄). Filtration and concentration furnished 224 mg (86%) of the acid fluoride as a yellow oil.

### Step 6

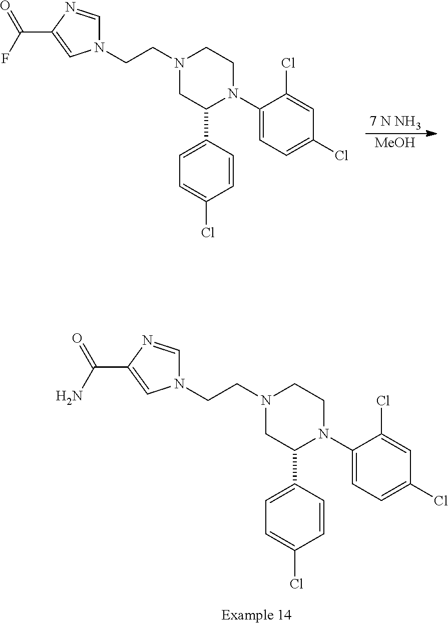

The acid fluoride (0.1 mmol) was taken up in 7 N NH₃ in MeOH. The solution was stirred at 25° C. for 18 h. The solution was concentrated. The residue was purified via thin-layer preparative chromatography (12.5/1 CH₂Cl₂/EtOH, SiO₂) which gave 36 mg (75%) of Example 14 as a colorless oil (LCMS (MH⁺) 478.3).

The following examples were prepared according to Step 6 of Scheme 6 using the appropriate amine (Table 2).

TABLE 2

| | | | LCMS |
|---|---|---|---|
| Amine | Ex. | Structure | (MH⁺) |
| 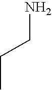 | 15 | 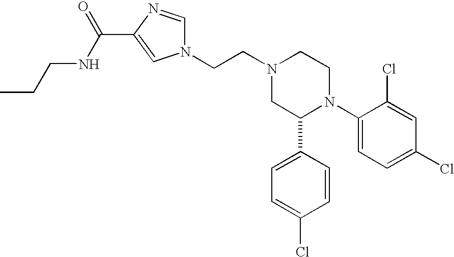 | 520.3 |
| 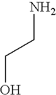 | 16 | 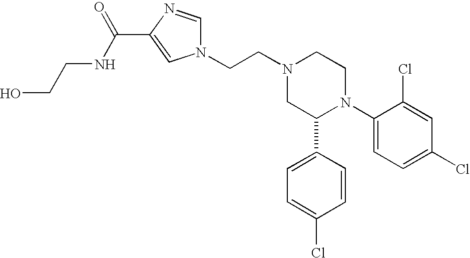 | 524.3 |
| 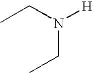 | 17 | 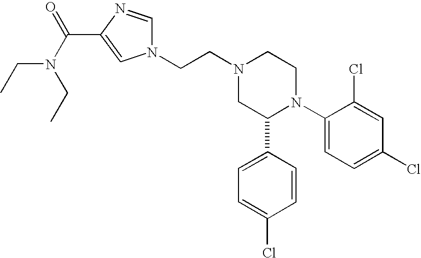 | 534.3 |

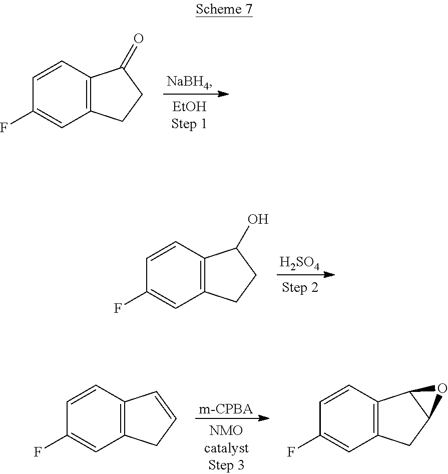

### Step 1:

To a solution of 5-fluoro-2,3-dihydro-1H-inden-1-one (0.66 g, 4.4 mmol) in EtOH (5 mL) was added NaBH₄ (216 mg, 5.75 mmol). The mixture was stirred at RT for 1 h. After that time, the mixture was concentrated. The residue was partitioned between water and EtOAc. The aqueous layer was extracted with EtOAc (3×). The combined organic layers were dried over Na₂SO₄, filtered and concentrated. The crude product was purified via flash chromatography (SiO₂: gradient elution, 100:0 to 75:25 hexanes:EtOAc) to afford the alcohol (ca 0.5 g).

### Step 2:

A solution of the alcohol (ca 0.5 g) in 1.5 M H₂SO₄ (15 mL) was heated to reflux for 1 h. The aqueous layer was neutralized with NaOH then extracted with EtOAc. The organic layer was dried over Na₂SO₄, filtered and concentrated. The crude product was purified via flash chromatography (SiO₂: hexanes) to afford the indene (ca 0.5 g).

### Step 3:

To a solution of the indene from step 2 (ca 0.5 g), (R,R)-(−)N,N′-Bis(3,5-di-tert-butylsalicylidene)-1,2-cyclohexanediaminomanganese(III) chloride (Aldrich) (118 mg, 0.19 mmol) and NMO (2.2 g, 18.5 mmol) in CH₂Cl₂ (15 mL) at −78° C. was added in two portions m-CPBA (1.69 g, 7.4 mmol). The resultant solution was stirred at that temperature for 2 h. At that time, a solution of dimethyl sulfide (1.04 g, 16.7 mmol) in CH₂Cl₂ (5 mL) at −78° C. was added. The solution was allowed to warm to RT and 3 N NaOH (aq.) (60 mL) was added. The aqueous layer was extracted with CH₂Cl₂ and the organic layer was washed with water, dried over Na₂SO₄, filtered and concentrated. The crude product was purified via flash chromatography (SiO₂: gradient elution, 100:0 to 80:20 hexanes:EtOAc) to afford the epoxide (major enantiomer pictured above; ca 50% ee).

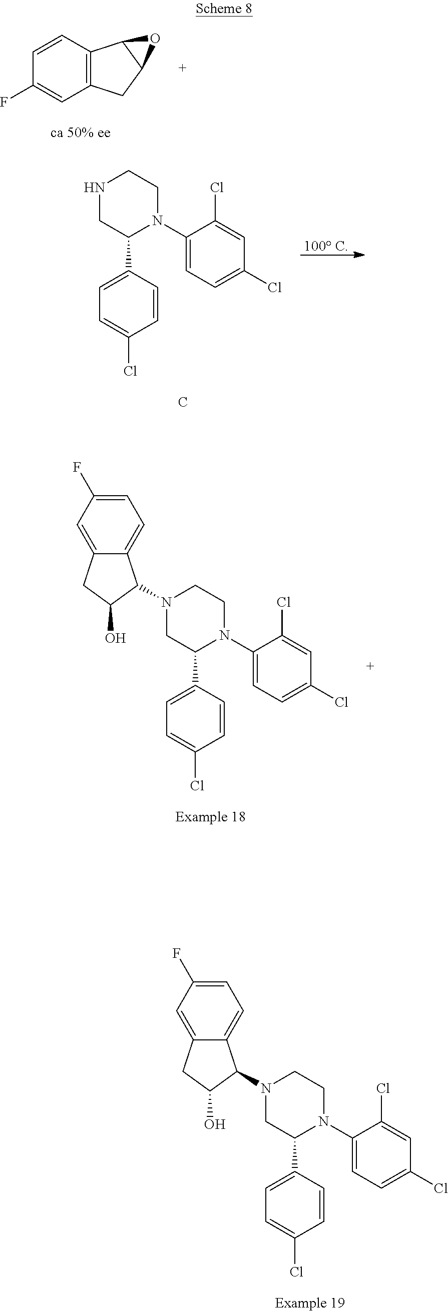

To a pressure tube containing the epoxide (ca 0.5 g) was added the piperazine C (250 mg, 0.73 mmol). The tube was sealed and the mixture was heated to 100° C. with stirring. After 16 h, the residue was cooled to RT was purified via flash chromatography (SiO₂: gradient elution, 100:0 to 80:20 hexanes:EtOAc) to afford Example 18 (106 mg): LCMS (MH⁺) 491.3, and Example 19 (30 mg): LCMS (MH⁺) 491.3.

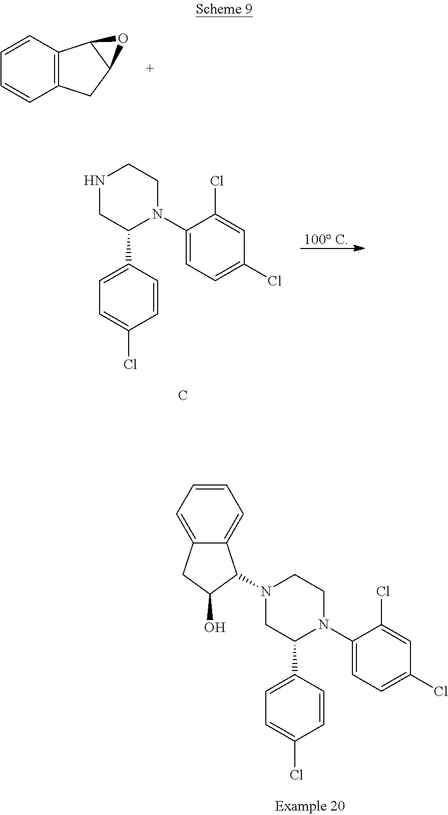

Example 20 was prepared using a procedure similar to that described in Scheme 8 except (1R, 2S)-indene oxide (98% ee) (Tet. Lett. 1995, 36, 5457-5460) was used instead of 5-fluoroindene oxide: LCMS (MH⁺) 475.3.

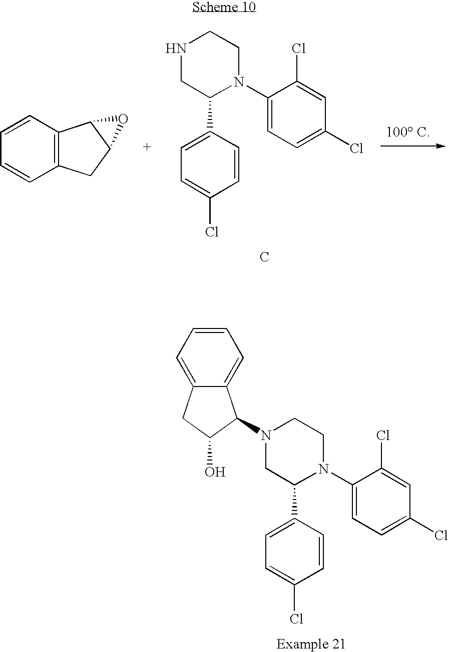

Example 21 was prepared using a procedure similar to that described in Scheme 8 except (1S, 2R)-indene oxide (98% ee) (Tet. Lett. 1995, 36, 5457-5460) was used instead of 5-fluoroindene oxide: LCMS (MH⁺) 475.3.

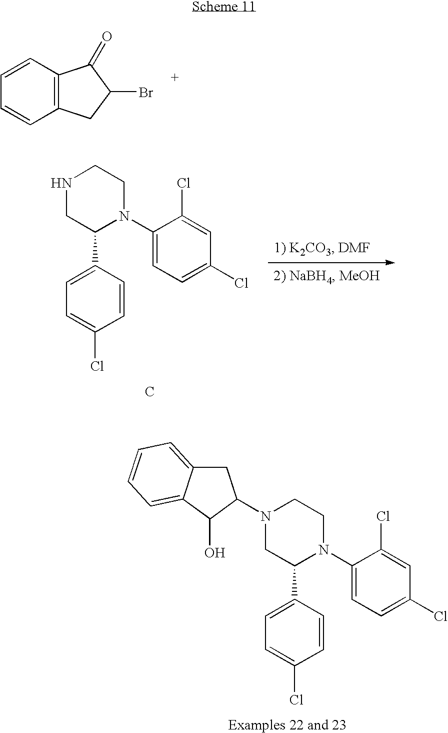

To a solution of the piperazine C (100 mg, 0.30 mmol) in DMF (5 mL) was added the bromide (63 mg, 0.30 mmol). The solution was stirred at RT for 2 days. The solution was partitioned between water and EtOAc. The organic layer was washed with brine, dried over Na₂SO₄, filtered and concentrated. The residue was dissolved in a mixture of THF and MeOH (5:2, 7 mL). To the solution was added NaBH₄ (11 mg, 0.30 mmol). The mixture was stirred at RT for 48 h. Water was added and the mixture was extracted with CH₂Cl₂. The organic layer was dried over Na₂SO₄, filtered and concentrated. The crude product was purified by preparative TLC (SiO₂: 1:1 hexanes:EtOAc) followed by separation of the diastereomers by HPLC (semi prep Chiralcel OD column: 90:10 hexanes:iPrOH) to afford the diastereomers Example 22 (2 mg): LCMS (MH⁺) 475.3 and Example 23 (1 mg): LCMS (MH⁺) 475.3.

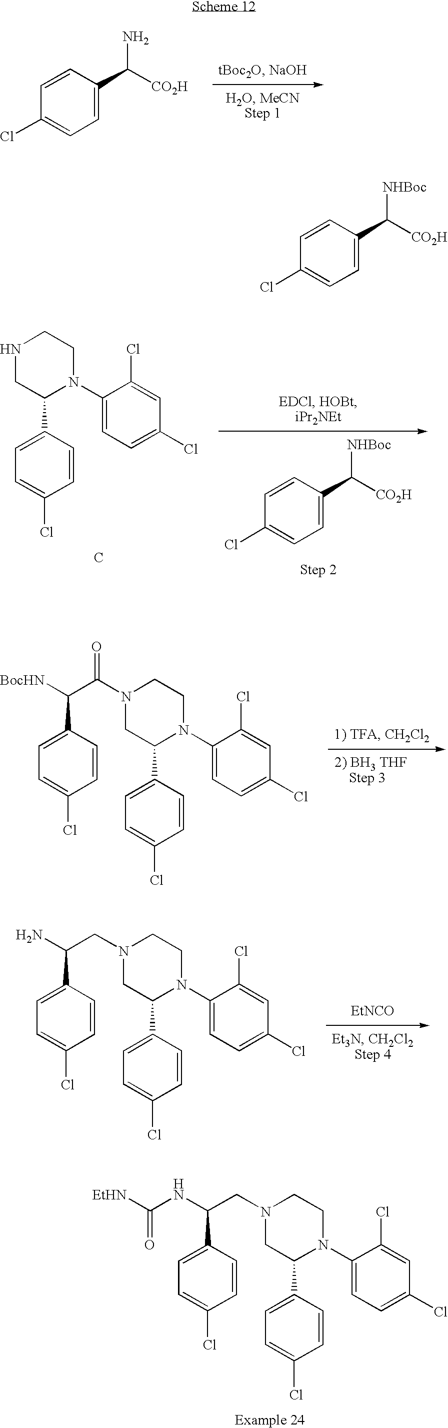

### Step 1:

A solution of (R)-2-amino-2-(4-chlorophenyl)acetic acid (1.0 g, 5.4 mmol), di-tert-butyl dicarbonate (1.2 g, 5.4 mmol) and NaOH (450 mg, 11 mmol) in water and MeCN (4:3) was stirred at RT overnight. The solution was acidified by the addition of 1 N HCl (aq.). The solution was extracted with CH₂Cl₂. The organic layer was dried over Na₂SO₄, filtered and concentrated in vacuo to afford the acid (1.4 g).

### Step 2:

To a solution of piperazine C (300 mg, 0.88 mmol) in MeCN (1.5 mL) was added EDCI (253 mg, 1.32 mmol), HOBt (178 mg, 1.32 mmol), iPr₂NEt (122 mg, 0.96 mmol) and the acid from step 1 (300 mg, 1.1 mmol). The solution was stirred at RT for 48 h. After that time, the solution was concentrated and purified via flash chromatography (SiO₂: gradient elution 100:0 to 85:15 hexanes:EtOAc) to afford the amide (600 mg).

### Step 3:

To a solution of the amide from step 2 (600 mg) in CH₂Cl₂ (10 mL) was added TFA. The solution was stirred at RT for 1 h. After that time, the solution was basified by the addition of excess sat. Na₂CO₃ (aq.). The mixture was extracted with CH₂Cl₂. The organic layer was dried over Na₂SO₄, filtered and concentrated. The resultant amido amine that was taken up in anhydrous THF (10 mL). To this solution was added borane THF complex (1 M in THF, 3 mL, 3 mmol). The resultant solution was heated to reflux with stirring for 3 h. After the reaction was complete, the solution was cooled to RT and excess hydrochloric acid was added (6 N). The mixture was stirred for 30 min. The mixture was then basified by the addition of excess sat NaHCO₃ (aq.) and the mixture was extracted with EtOAc (3×). The combined organic layers were dried over Na₂SO₄, filtered and concentrated to afford the amine that was used without further purification.

### Step 4:

To a solution of the amine (50 0.1 mmol) in CH₂Cl₂ (2 mL) was added ethyl isocyanate (11 mg, 0.15 mmol) and iPr₂NEt (51 mg, 0.5 mmol). The solution was stirred at RT overnight. The solution was then concentrated and purified via preparative TLC [95:5:0.5 CH₂Cl₂:MeOH:concentrated NH₄OH (aq.)] to afford Example 24 (ca 44 mg): LCMS (MH⁺) 567.3.

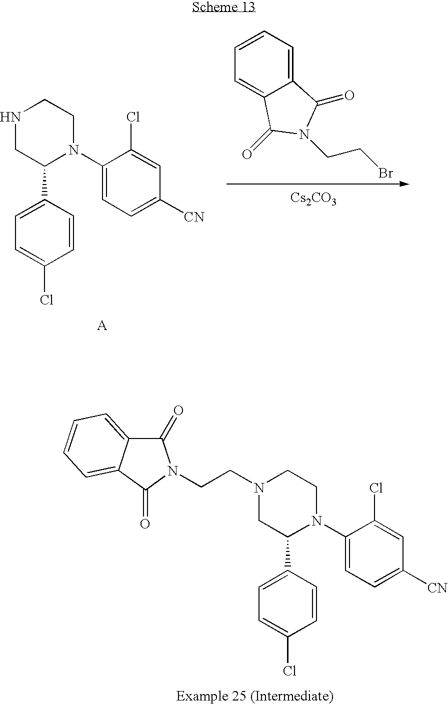

To a solution of the bromide (2.7 g, 10.8 mmol) and the piperazine A (3 g, 9.0 mmol) in MeCN in a pressure tube was added Cs₂CO₃ (4 g). The pressure tube was sealed and the mixture was heated to 80° C. with stirring. After 16 h, the mixture was cooled to RT and concentrated in vacuo. The residue was then partitioned between CH₂Cl₂ and water. The aqueous layer was extracted with CH₂Cl₂ (3×). The combined organic layers were dried over Na₂SO₄, filtered and concentrated. The crude product was purified via flash chromatography (SiO₂: gradient elution 100:0 to 1:1 hexanes:EtOAc) to afford Example 25 (1.42 g) as a light yellow solid: LCMS (MH⁺) 505.3.

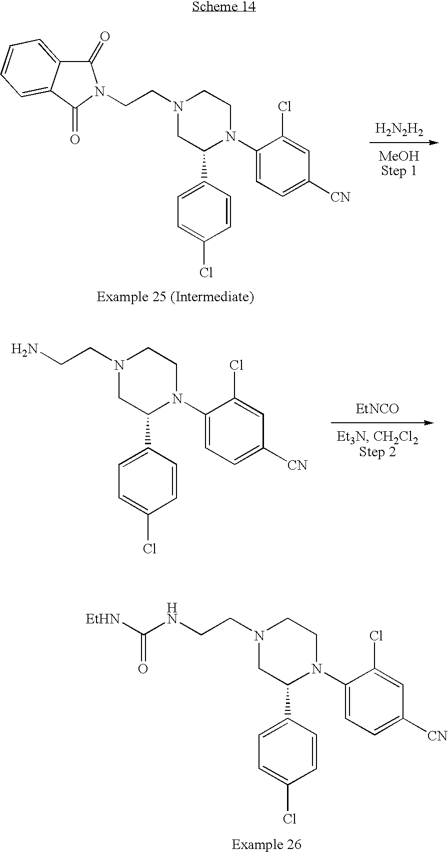

### Step 1:

To a solution of Example 25 (1.4 g, 2.81 mmol) in MeOH was added hydrazine (360 mg, 11.2 mmol). The resultant solution was heated to reflux with stirring for 3 hours. After the reaction was determined to be complete, the solution was concentrated in vacuo. To the residue was added EtOAc, the solids were removed via filtration, and the solvent was removed in vacuo. The crude product was purified via flash chromatography [SiO₂: gradient elution 100:0:0:0 96:4:0.2:0.2 CH₂Cl₂:MeOH:7 N NH₃ (in MeOH): conc NH₄OH (aq.)] to afford the intermediate amine (ca. 700 mg).

### Step 2:

Example 26 was prepared using a method similar to that described in Scheme 12 Step 4, except the amine from step 1 of this scheme was used:

LCMS (MH⁺) 446.2.

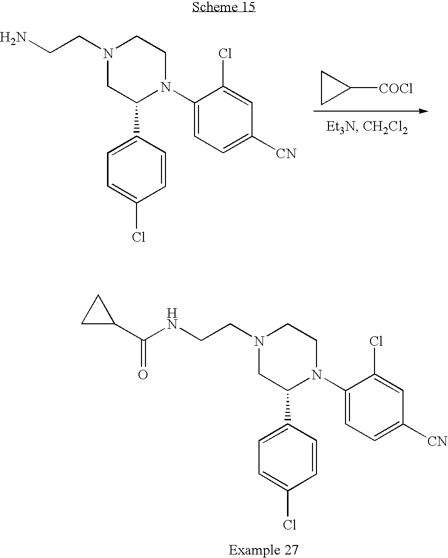

To a solution of the amine from Scheme 14 step 1 (50 mg, 0.1 mmol) in CH₂Cl₂ (10 mL) was added Et₃N (15 mg, 0.15 mmol) and cyclopropanecarbonyl chloride (13 mg, 0.12 mmol). The resultant solution was heated to reflux with stirring for 16 h. After that time, the solution was concentrated and purified via preparative TLC [SiO₂: 95:5:0.5 CH₂Cl₂:MeOH:conc NH₄OH (aq.)] to afford Example 27 (ca. 32 mg): LCMS (MH⁺) 443.2.

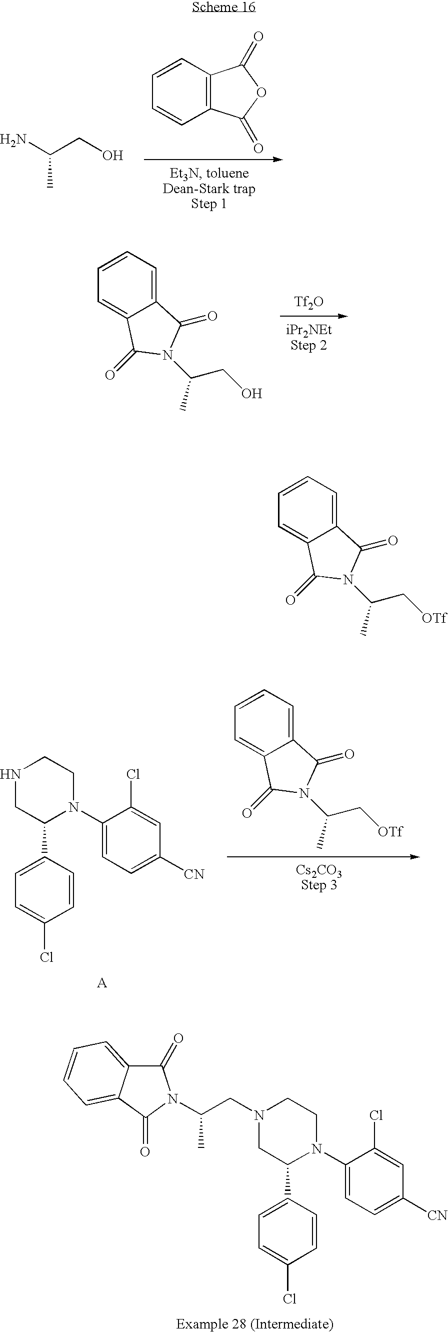

### Step 1:

To a solution of 2-(S)-amino-1-propanol (2.0, 27 mmol) in toluene in a round bottom flask was added Et₃N (0.37 mL, 2.65 mmol) and phthalic anhydride (3.9 g, 27 mmol). A Dean-Stark trap was attached and the solution was heated to reflux for 24 h with stirring. After the reaction was determined to be complete, the solution was concentrated in vacuo and the crude product was purified via flash chromatography (SiO₂: gradient elution 100:0 to 0:100 hexanes:EtOAc) to afford the alcohol (4.9 g) as a white solid.

### Step 2:

To a solution of the alcohol from step 1 (500 mg, 2.4 mmol) in CH₂Cl₂ at −25° C. was added iPr₂NEt (465 mg, 3.6 mmol) followed by trifluoromethanesulfonic anhydride (745 mg, 2.6 mmol). The resultant solution was stirret at −25° C. for 1 h. After that time, the solution was concentrated and the residue was filtered through a silica gel plug using 1:1 EtOAc:hexanes as the elutant. The solvent was concentrated to afford the triflate (ca 300 mg).

### Step 3:

To a solution of the piperazine A (500 mg) in DMF (5 mL) at 0° C. was added the triflate from step 2 (ca 300 mg) followed by iPr₂NEt (0.6 mL). The resultant solution was allowed to slowly warm to RT and stir for 48 h. The solution was then concentrated in vacuo and the residue was partitioned between water and CH₂Cl₂. The organic layer was dried over Na₂SO₄, filtered and concentrated. The crude product was purified via flash chromatography (SiO₂: gradient elution 100:0 to 50:50 hexanes:EtOAc) to afford Example 28 (470 mg) as a light yellow solid: LCMS (MH⁺) 519.3.

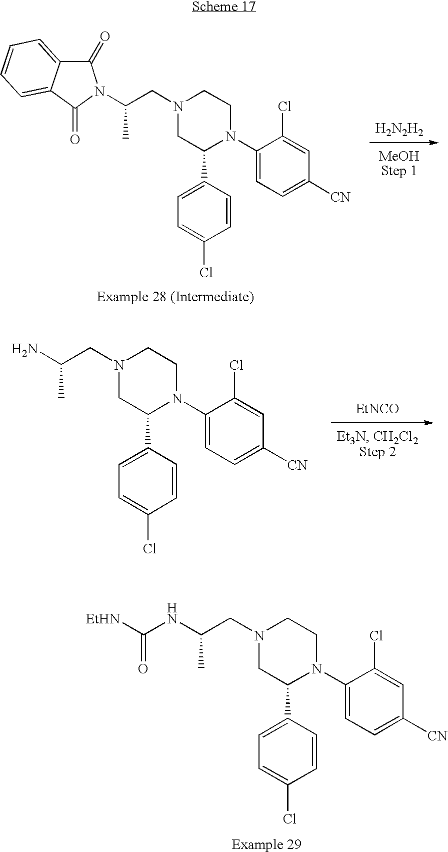

### Step 1:

The amine was prepared using a similar method to that described in Scheme 14 Step 1, but the intermediate of Example 28 was used.

### Step 2:

Example 29 was prepared using a similar method to that described in Scheme 12 step 4, except the amine from Step 1 of this scheme was used:

LCMS (MH⁺) 460.3.

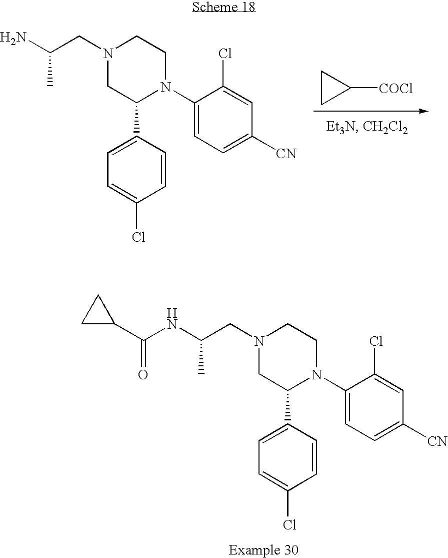

Example 30 was prepared using a similar method to that described in Scheme 15, except the amine from Scheme 17 step 1 was used: LCMS (MH⁺) 457.3.

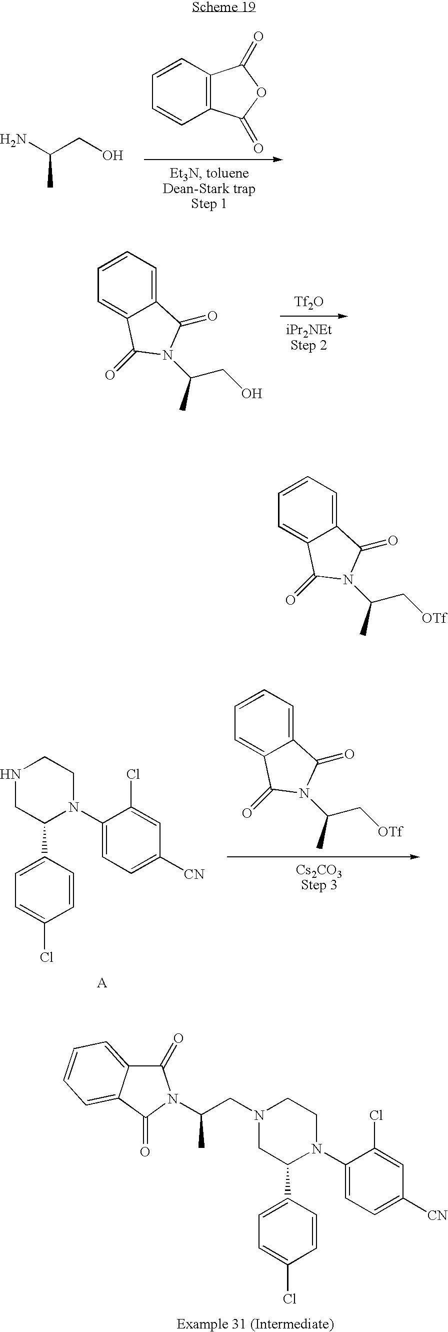

Example 31 was prepared using a method similar to that described in Scheme 16 except 2-(R)-amino-1-propanol was used: LCMS (MH⁺) 519.3.

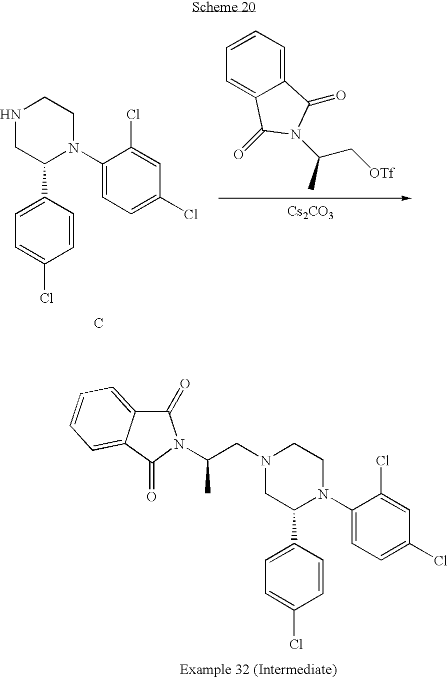

Example 32 was prepared using a method similar to that described in Scheme 16 step 3, except piperazine C was used: LCMS (MH⁺) 528.3.

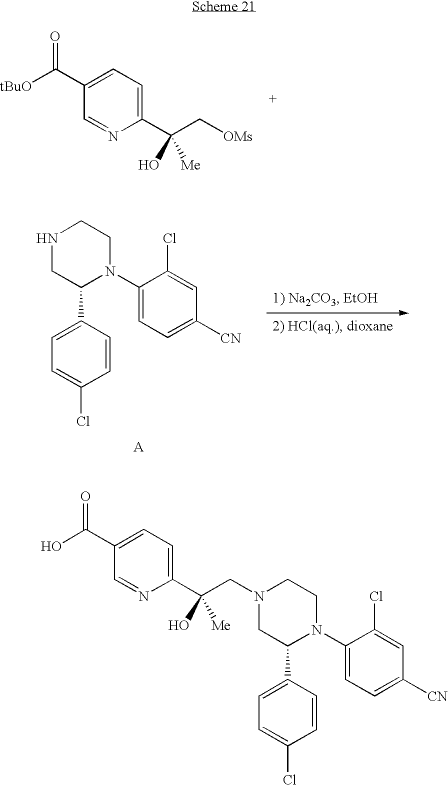

To a solution of the mesylate from Scheme 3 (500 mg, 1.50 mmol) and the piperazine A (385 mg, 1.16 mmol) in EtOH (5 mL) in a pressure tube was added Na₂CO₃ (160 mg, 1.50 mmol). The tube sealed and the mixture was heated to 80° C. with stirring for 16 h. The mixture was then cooled to RT, transferred to a round bottom flask and concentrated in vacuo. The residue was partitioned between EtOAc and water. The aqueous layer was extracted with EtOAc (3×). The combined organic layers were washed with brine, dried over Na₂SO₄, filtered and concentrated. The crude product was purified via flash chromatography (SiO₂: gradient elution, 100:0 to 50:50 hexanes:EtOAc) to afford the ester (580 mg). The ester was taken up in a solution of HCl (aq.) (4N, 3 mL) and HCl (dioxane) (4 N, 20 mL). The resultant solution was heated to 70° C. for 2 h. The solution was concentrated and the carboxylic acid HCl salt was used without further purification.

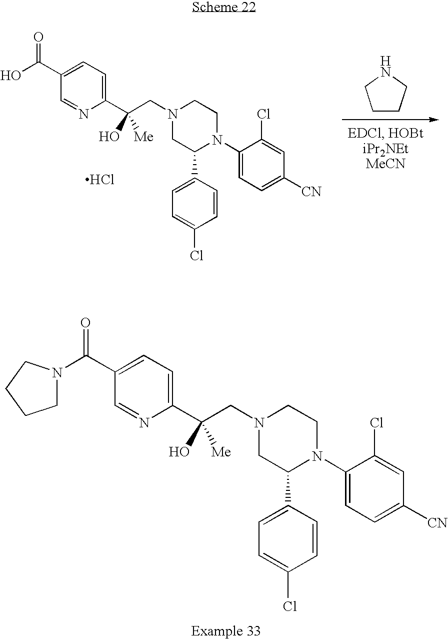

To a solution of the carboxylic acid from Scheme 21 (120 mg, 0.23 mmol) and iPr₂NEt (130 mg, 0.99 mmol) in MeCN (1.5 mL) was added EDCI (97 mg, 0.50 mmol), pyrrolidine (50 mg, 0.70 mmol) and HOBt (68 mg, 0.50 mmol). The resultant mixture was stirred at RT for 2.5 days. After that time, the mixture was concentrated in vacuo and the residue was partitioned between EtOAc and 1 M NaOH (aq.). The aqueous layer was extracted with EtOAc (3×). The combined organic layers were washed with brine, dried over Na₂SO₄, filtered and concentrated. The crude product was purified via preparative TLC (SiO₂: 1:1 Acetone:Hexanes) to afford Example 33 (31 mg): LCMS (MH⁺) 564.3.

TABLE 3
The following examples were prepared using a method similar to that described in Scheme 22.

| | | | LCMS |
|---|---|---|---|
| Amine | Example | Structure | (MH⁺) |
| 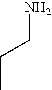 | 34 | 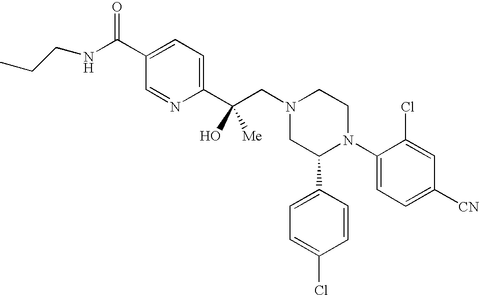 | 552.3 |
| 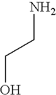 | 35 | 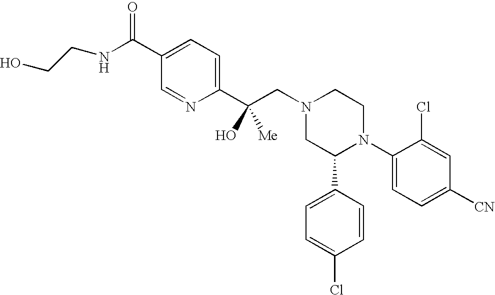 | 554.3 |
| 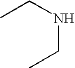 | 36 | 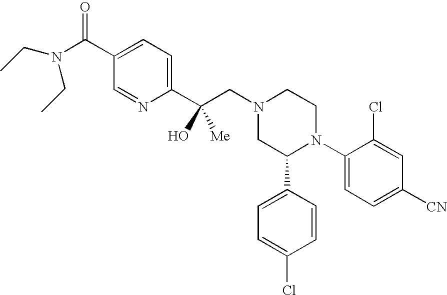 | 566.3 |
| 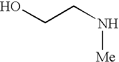 | 37 | 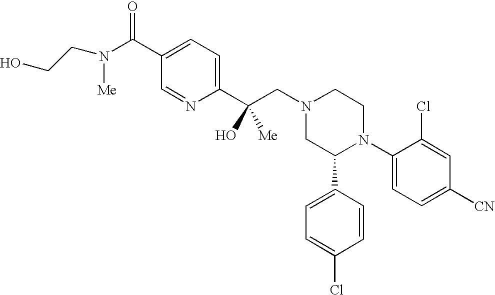 | 568.3 |

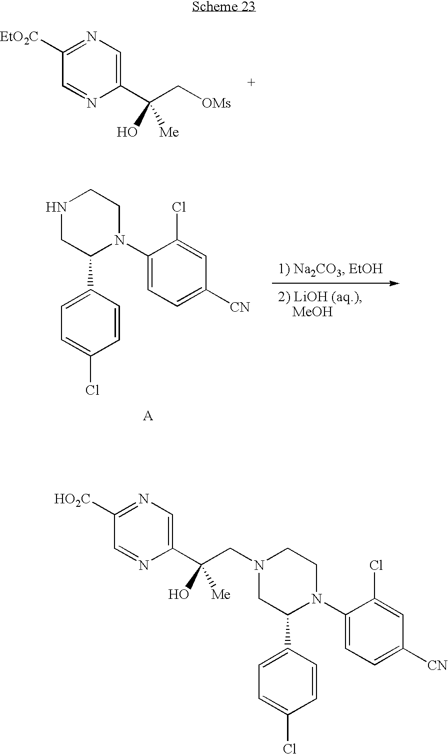

To a solution of the mesylate from Scheme 4 (600 mg, 1.97 mmol) in EtOH (15 mL) in a pressure tube was added the piperazine (545 mg, 1.64 mmol) and Na₂CO₃ (226 mg, 2.13 mmol). The pressure tube was sealed and the mixture was heated to 80° C. with stirring. After 16 h, the mixture was cooled to RT and concentrated in vacuo. The residue was then partitioned between water and EtOAc and the aqueous layer was extracted with EtOAc (3×). The combined organic layers were washed with brine, dried over Na₂SO₄, filtered and concentrated. The crude product was purified via flash chromatography (SiO₂: gradient elution 100:0 to 30:70 hexanes:EtOAc) to afford the ester (ca 450 mg).

To a solution of the ester (450 mg, 0.83 mmol) in MeOH (15 mL) was added a solution of LiOH (aq.) (2M, 2.5 mmol). The resultant mixture was stirred at RT for 2.5 days. The mixture was then concentrated. Water was added and the mixture was adjusted to pH 6 by the addition of 1 M HCl (aq.). The aqueous layer was then extracted with EtOAc (3×). The combined organic layers were washed with brine, dried over Na₂SO₄, filtered and concentrated to afford the carboxylic acid (402 mg) that was used without further purification.

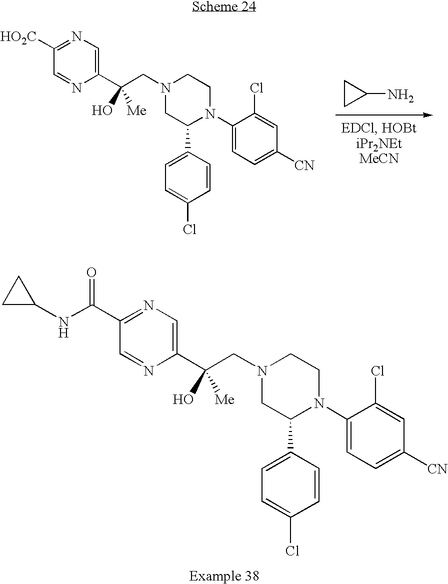

To a solution of the carboxylic acid from Scheme 23 (100 mg, 0.20 mmol) in MeCN (1 mL) was added cyclopropyl amine (23 mg, 0.40 mmol), EDCI (77 mg, 0.40 mmol), HOBt (54 mg, 0.40 mmol) and iPr₂NEt (52 mg, 0.40 mmol). The resultant mixture was stirred at RT overnight. After that time, the mixture was concentrated in vacuo and the residue was partitioned between EtOAc and 1 M NaOH (aq.). The aqueous layer was extracted with EtOAc (3×). The combined organic layers were washed with brine, dried over Na₂SO₄, filtered and concentrated. The crude product was purified via preparative TLC (SiO₂: 1:1 EtOAc:Hexanes) to afford Example 38 (28 mg): LCMS (MH⁺) 551.3.

TABLE 4
The following examples were prepared using a method similar to that described in Scheme 24.

| | | | LCMS |
|---|---|---|---|
| Amine | Example | Structure | (MH⁺) |
| 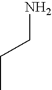 | 39 | 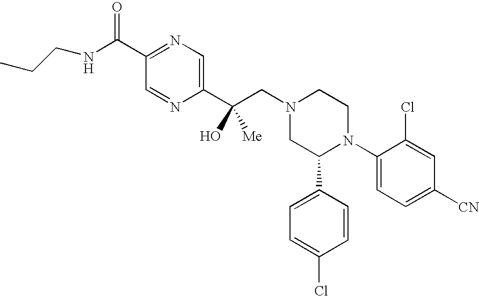 | 553.3 |
| 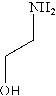 | 40 | 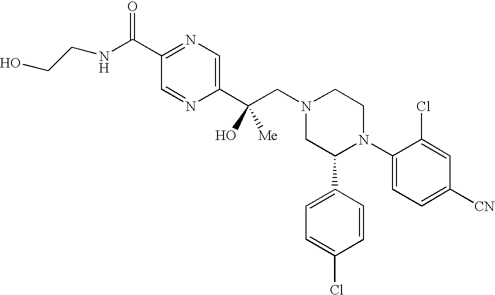 | 555.3 |
| 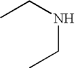 | 41 | 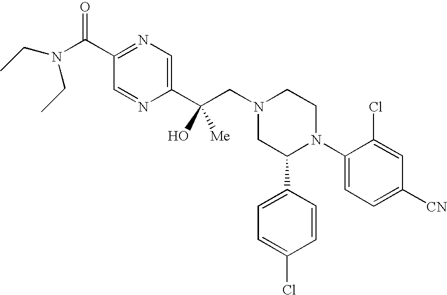 | 567.3 |

### Examples

The compounds of Formula (I) shown in the following table were prepared according to one or more methods reported above.

Structure

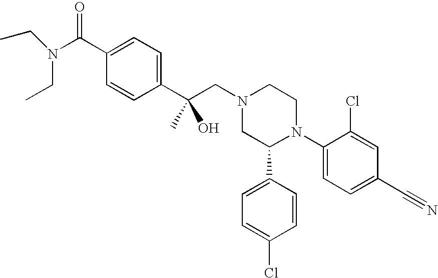

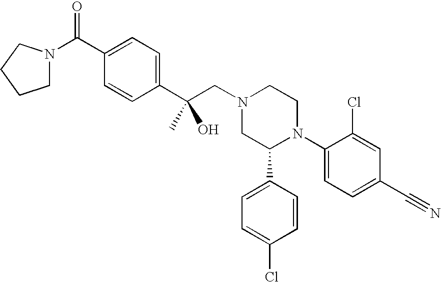

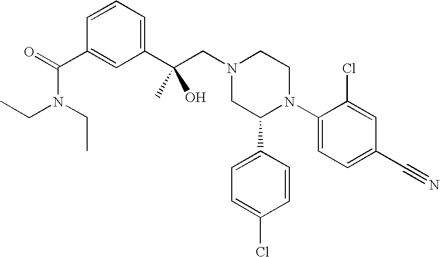

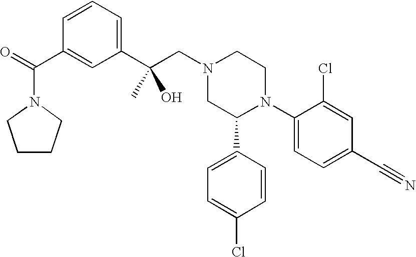

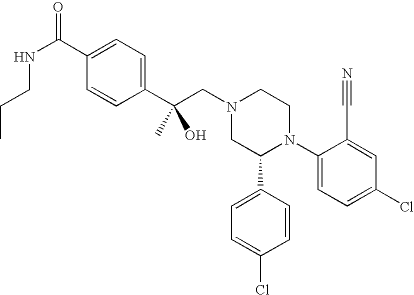

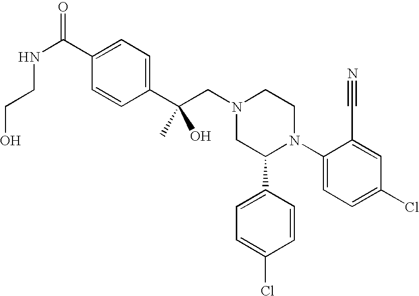

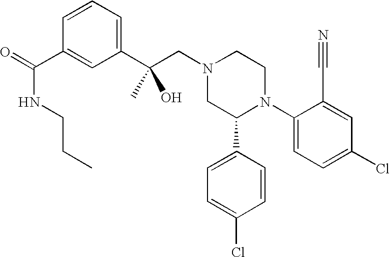

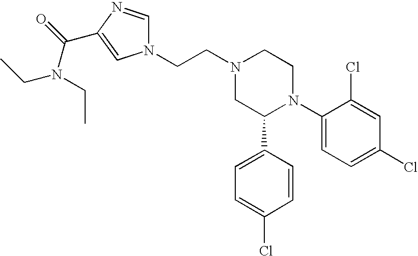

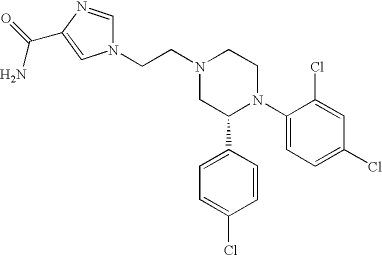

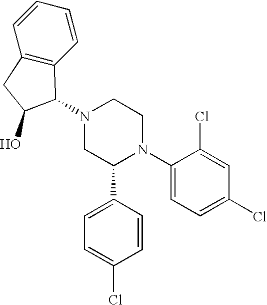

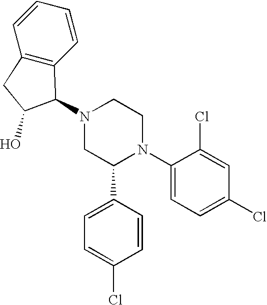

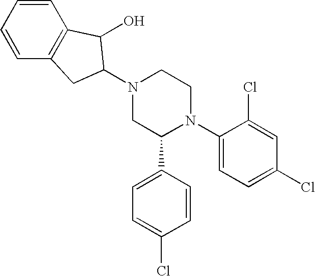

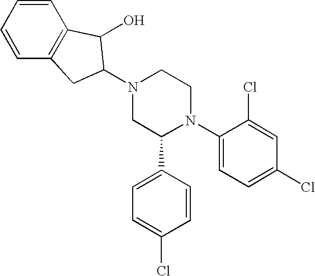

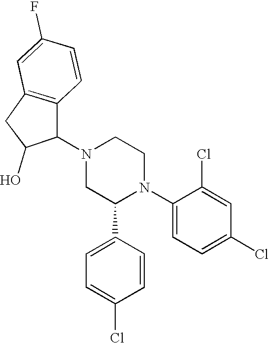

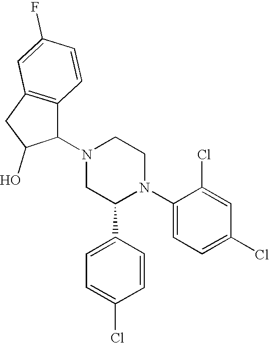

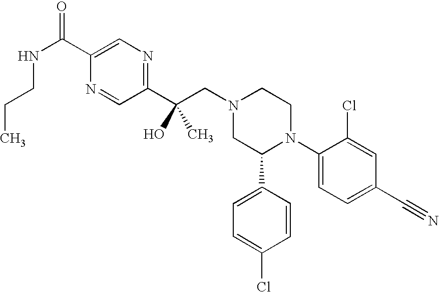

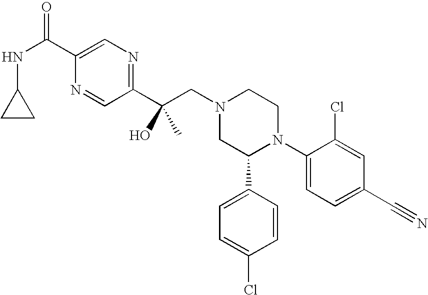

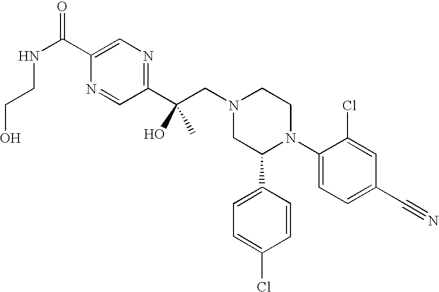

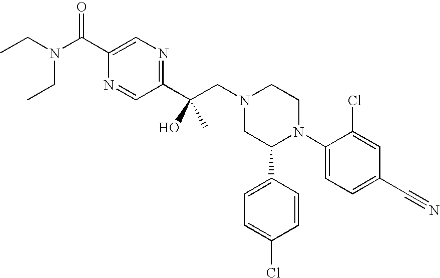

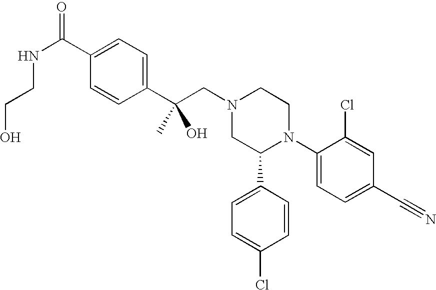

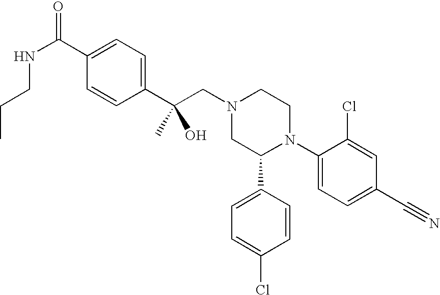

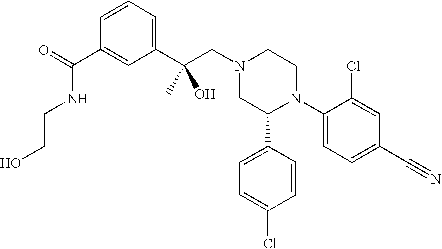

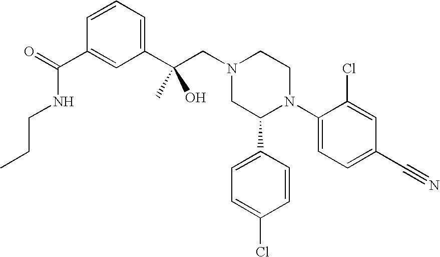

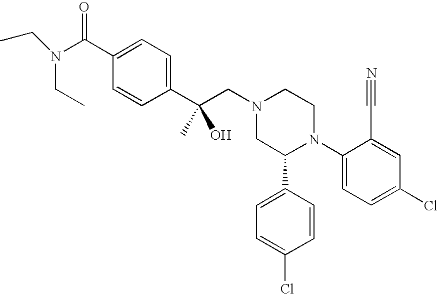

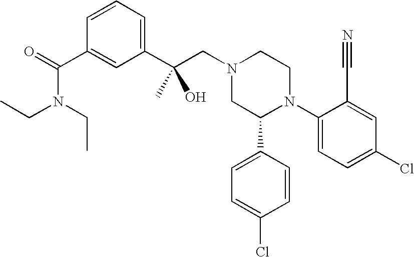

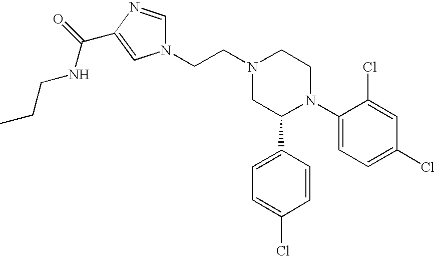

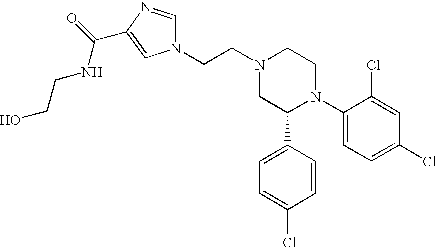

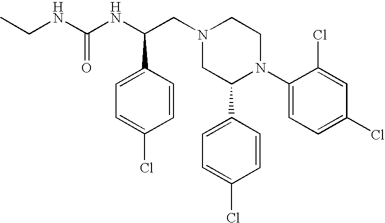

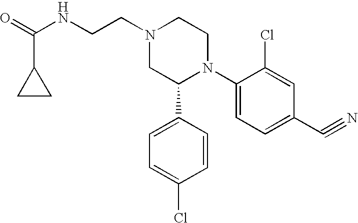

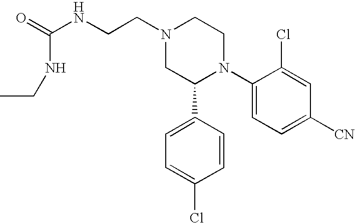

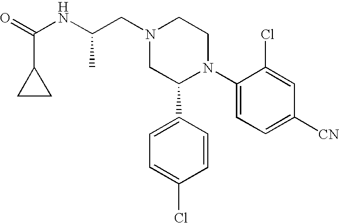

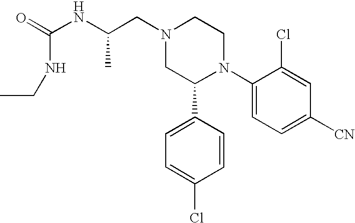

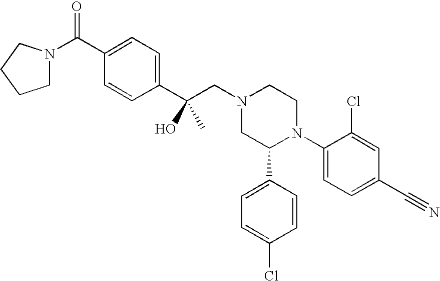

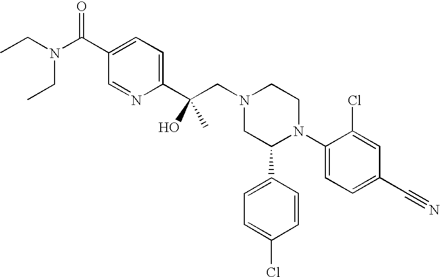

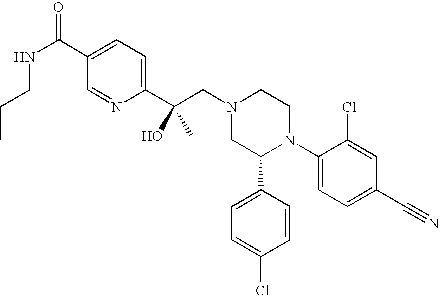

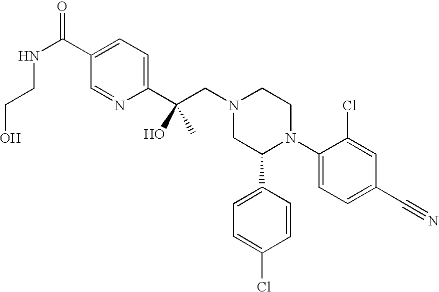

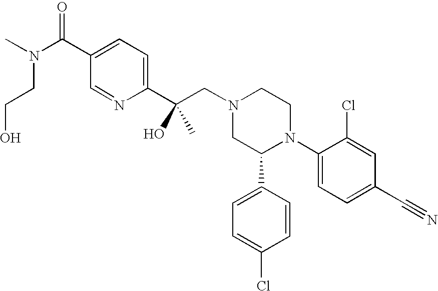

1. A compound of Formula (I): 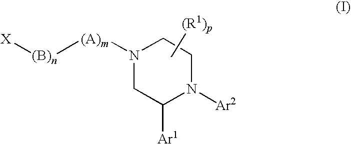
or a pharmaceutically acceptable salt, thereof, wherein:
Ar¹ and Ar² are independently aryl or heteroaryl,
wherein each of Ar¹ and Ar² is substituted with one or more groups independently selected from Y¹;
with the proviso that when Ar² is pyridine or pyrimidine, a nitrogen of said pyridine or pyrimidine is not in the para position relative to the point of attachment to the piperazine ring;
n and m are independently 0 or 1;
A is selected from the group consisting of —C(O)—, —S(O)₂—, —C(═N—OR²)—. and —(C(R²)₂)_{q}— wherein q is 1, 2, or 3;
B is selected from the group consisting of —N(R²)—, —C(O)—, and —(C(R³)₂)ᵣ— wherein r is 1 or 2,
with the proviso that when B is —C(O)—, then A is —C(O)— or —(C(R²)₂)_{q}—:
X is selected from the group consisting of:
—C(O)N(R⁶)₂, —C(O)-cycloalkyl, —C(O)-heterocycloalkyl, aryl substituted with one or more groups independently selected from —C(O)N(R⁶)₂, heteroaryl substituted with one or more groups independently selected from —C(O)N(R⁶)₂, and benzo-fused cycloalkyl-, wherein the cycloalkyl portion of said benzo-fused cycloalkyl- is substituted with at least one —OH group, and wherein the aryl portion of said benzo-fused cycloalkyl- is unsubstituted or substituted with one or more groups independently selected from Z,
with the proviso that, when X is —C(O)N(R⁵)₂. —C(O)-cycloalkyl or —C(O)-heterocycloalkyl, then n=1 and B is —NR²—;
each R¹ is independently selected from the group consisting of alkyl, haloalkyl, -alkylene-NR²R⁵, -alkylene-OR², alkylene-N₃, -alkylene-CN, and alkylene-O—S(O)₂-alkyl; or
two R¹ groups attached to the same ring carbon atom form a carbonyl group;
p is 0, 1, 2, 3, or 4;
each R² is independently H, alkyl, aryl, heteroaryl, cycloalkyl, or heterocycloalkyl,
wherein each of said aryl heteroaryl, cycloalkyl, and heterocycloalkyl of R² is unsubstituted or optionally substituted with one or more groups independently selected from Y¹;
each R³ is independently selected from the group consisting of H, alkyl, unsubstituted aryl, aryl substituted with one or more Y¹ groups, —OR², -alkylene-O-alkyl, and -alkylene-OH;
each R⁴ is independently selected from the group consisting of H, alkyl, aryl, —C(O)—O-alkyl, —C(O)-alkyl, —C(O)-aryl, —C(O)-heteroaryl, —S(O)₂alkyl, —S(O)₂aryl, —S(O)₂heteroaryl, and —S(O)₂heterocycloalkyl,
wherein each of said aryl, the aryl portion of said —C(O)-aryl, the aryl portion of said —S(O)₂aryl of R⁴, and the heteroaryl portion of said —C(O)-heteroaryl, and —S(O)₂heteroaryl, is unsubstituted or substituted with one or more groups independently selected from Y¹;
each R⁵ is independently selected from the group consisting of H, alkyl, aryl. —S(O)₂-alkyl, —S(O)₂-cycloalkyl, —S(O)₂-aryl, —S(O)₂-heteroaryl, —S(O)₂-heterocycloalkyl, —C(O)—N(R²)₂, —C(O)-alkyl, —C(O)-cycloalkyl, —C(O)-aryl, —C(O)-heteroaryl, —C(O)-heterocycloalkyl, and -alkylene-OH,
wherein each of said aryl, the aryl portions of said —S(O)₂-aryl and —C(O)-aryl, and the heteroaryl portions of said —S(O)₂-heteroaryl and said —C(O)-heteroaryl of R⁵ is unsubstituted or substituted with one or more Z groups;
each Y¹ is independently selected from the group consisting of halo, —CN, alkyl, haloalkyl, cycloalkyl, heterocycloalkyl, heterocycloalkenyl, aryl, -alkylene-aryl, heteroaryl, —O-alkyl, —O-haloalkyl, —O-aryl, —O-heteroaryl, —O-cycloalkyl, —O-heterocycloalkyl, —S-alkyl, —S-haloalkyl, —S-heteroaryl, —S-cycloalkyl, —S-heterocycloalkyl, —S(O)₂-alkyl, —S(O)₂-cycloalkyl, —S(O)₂-heterocycloalkyl, —S(O)₂-aryl, —S(O)₂-heteroaryl, -alkylene-CN, —C(O)-alkyl, —C(O)-aryl, —C(O)-haloalkyl, —C(O)-heteroaryl, —C(O)— cycloalkyl, —C(O)-heterocycloalkyl, —C(O)O-alkyl, —C(O)O-aryl, —C(O)O-haloalkyl, —C(O)O-heteroaryl, —C(O)O— cycloalkyl, —C(O)O-heterocycloalkyl, —N(R²)C(O)-alkyl, —N(R²)C(O)—N(R²)₂, —OH, -alkylene-OH, -alkylene-C(O)—O-alkyl, —O-alkylene-aryl, and —NR²R^{5 ,}
wherein each of said aryl, each -alkylene-aryl, each heteroaryl, each aryl portion of said —O-aryl, each heteroaryl portion of said —O-heteroaryl, each aryl portion of said —S-aryl, each heteroaryl portion of said —S-heteroaryl, each aryl portion of said —S(O)₂-aryl, each heteroaryl portion of said —S(O)₂-heteroaryl, each aryl portion of said —C(O)-aryl, each heteroaryl portion of said —C(O)-heteroaryl, each aryl portion of said —C(O)O-aryl, and each heteroaryl portion of said —C(O)O-heteroaryl of Y¹ are unsubstituted or substituted with one or more groups Z; or
two groups Y¹ form a —O—CH₂—O— group;
each R⁶ is independently selected from the group consisting of H, alkyl, haloalkyl, alkoxy, cycloalkyl, heterocycloalkyl, unsubstituted aryl, aryl substituted with one or more groups independently selected from Z, unsubstituted heteroaryl, heteroaryl substituted with one or more groups independently selected from Z, cycloalkyl, -alkylene-OH, -alkylene-O-alkyl, -alkylene-O-aryl, -alkylene-OC(O)-alkyl, -alkylene-OC(O)-aryl, -alkylene-OC(O)-heteroaryl, and alkylene-NR⁴R², or
two R⁶ groups, together with the nitrogen to which they are attached, form a heteroaryl, heterocycloalkyl, heterocycloalkenyl, or a benzo-fused heterocycloalkyl group; and
each Z is independently selected from the group consisting of alkyl, halo, haloalkyl, —OH, —O-alkyl, and —CN;
with the proviso that when A is —C(O)—, then each Y¹ on Ar¹ is independently selected from the group consisting of cycloalkyl, benzyl, aryl, —O-haloalkyl, —O-aryl, —O-cycloalkyl, —S-aryl, —S-haloalkyl, —S-cycloalkyl, —S(O)₂-alkyl, —S(O)₂-cycloalkyl, —S(O)₂-aryl, -alkylene-CN, —C(O)-aryl, —C(O)-haloalkyl, —C(O)— cycloalkyl, —C(O)O-aryl, —C(O)O-haloalkyl, —C(O)O-heteroaryl, —C(O)O -cycloalkyl, —C(O)O-heterocycloalkyl, -alkylene-C(O)—O-alkyl, and —O-alkylene-aryl, wherein each benzyl and each aryl portion of Y¹, and each aryl portion and each heteroaryl portion of said —O-aryl. said —S-aryl, said —S(O)₂-aryl, said —C(O)-aryl, said —C(O)O-aryl, —C(O)O-heteroaryl, —C(O)O-heterocycloalkyl, and —O-alkylene-aryl of Y¹, are unsubstituted or substituted with one or more groups independently selected from Z; or two groups Y¹ form a —O—CH₂—O-group.

2-36. (canceled)

37. A compound, or a pharmaceutically acceptable salt, thereof, selected from:

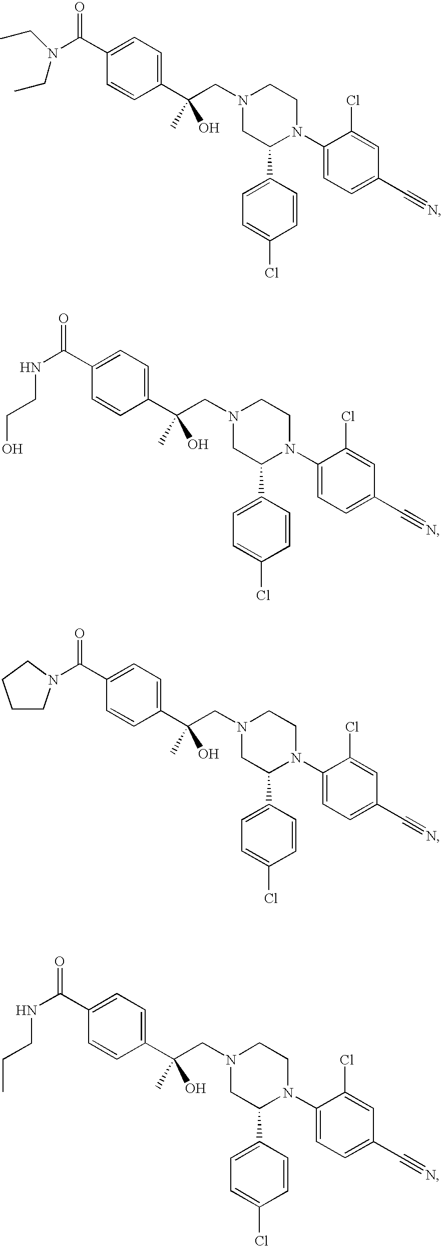

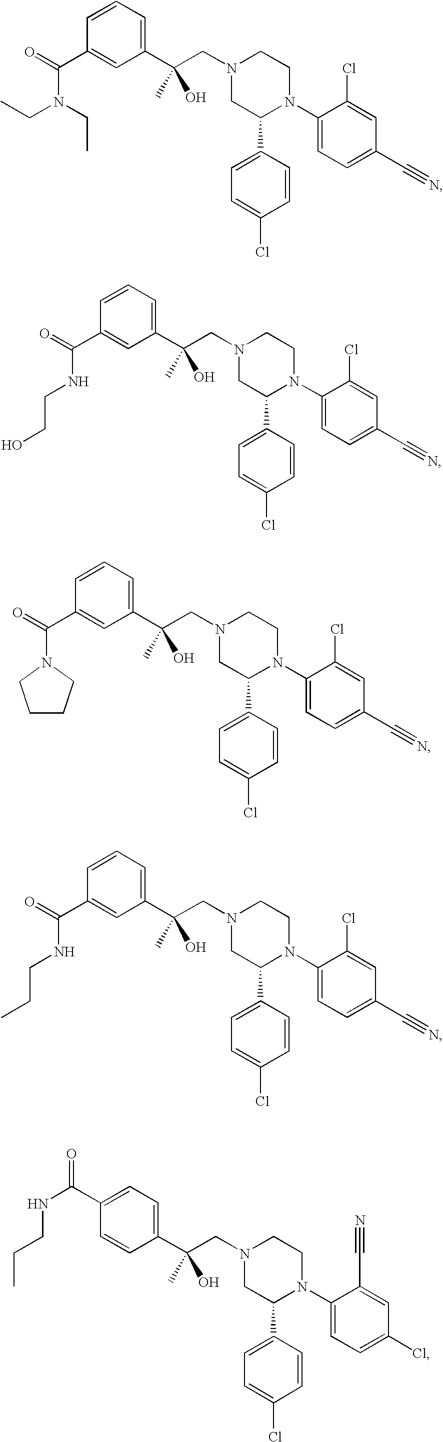

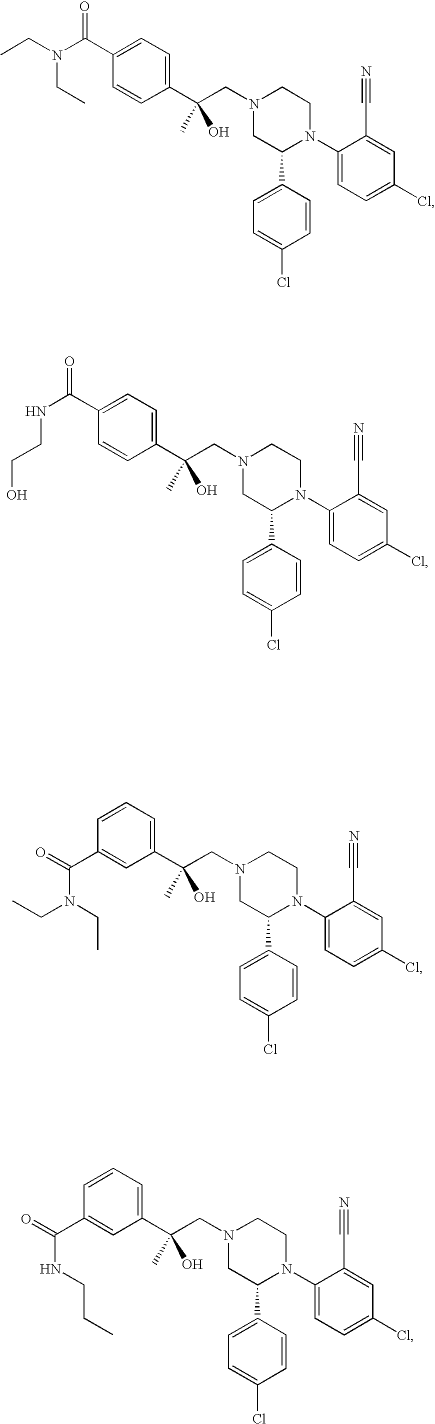

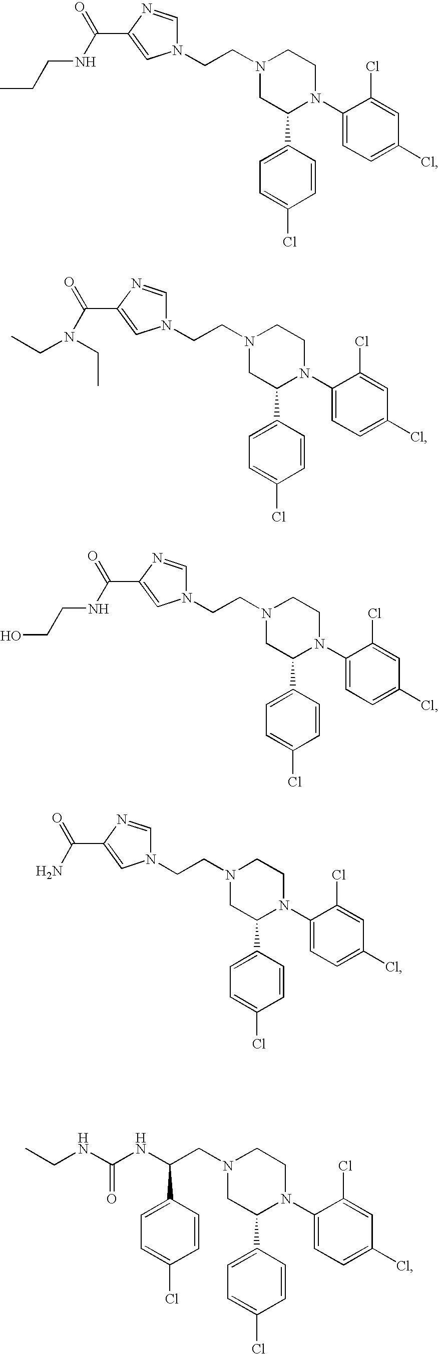

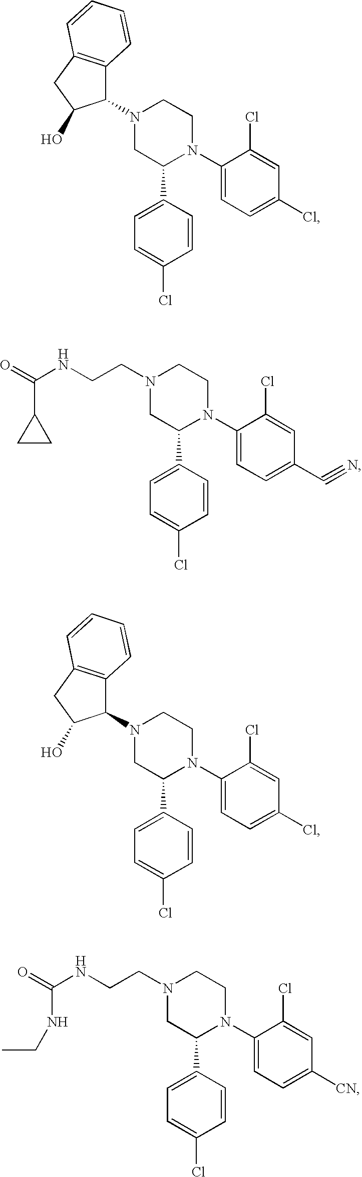

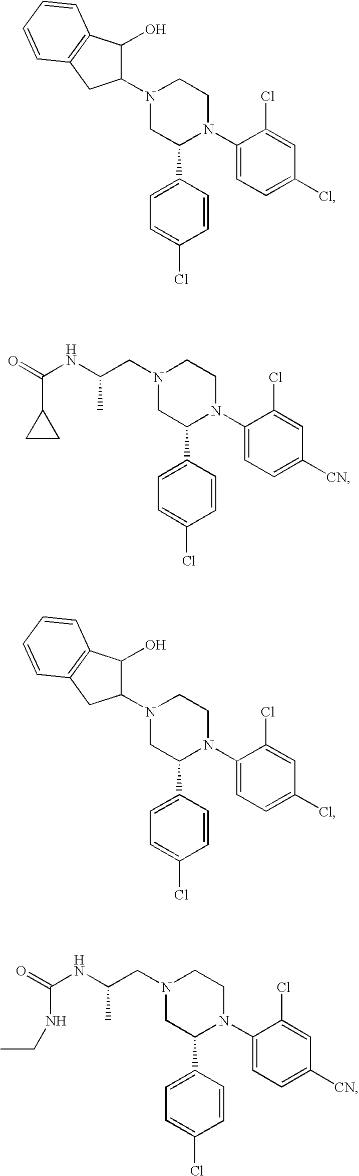

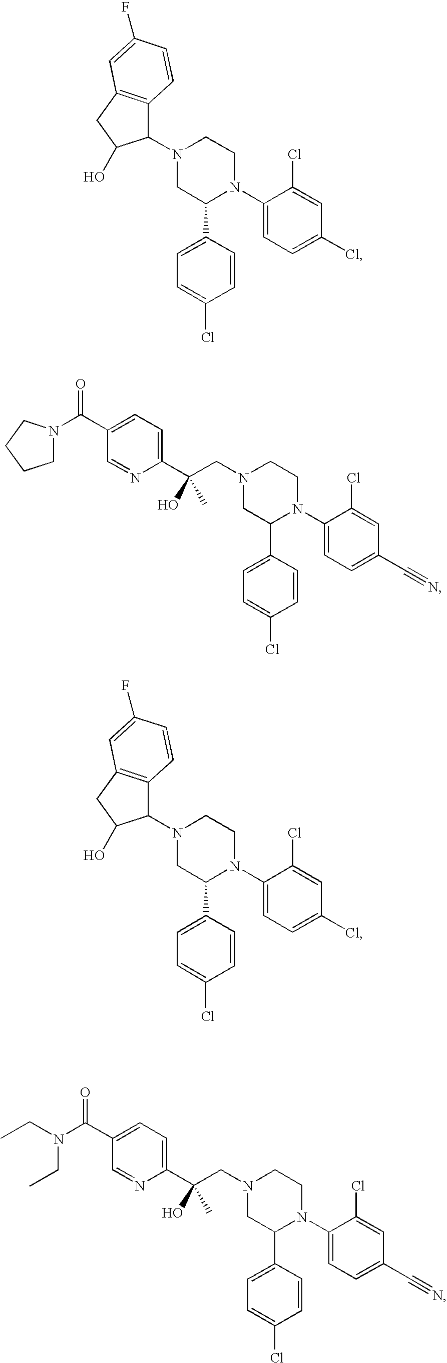

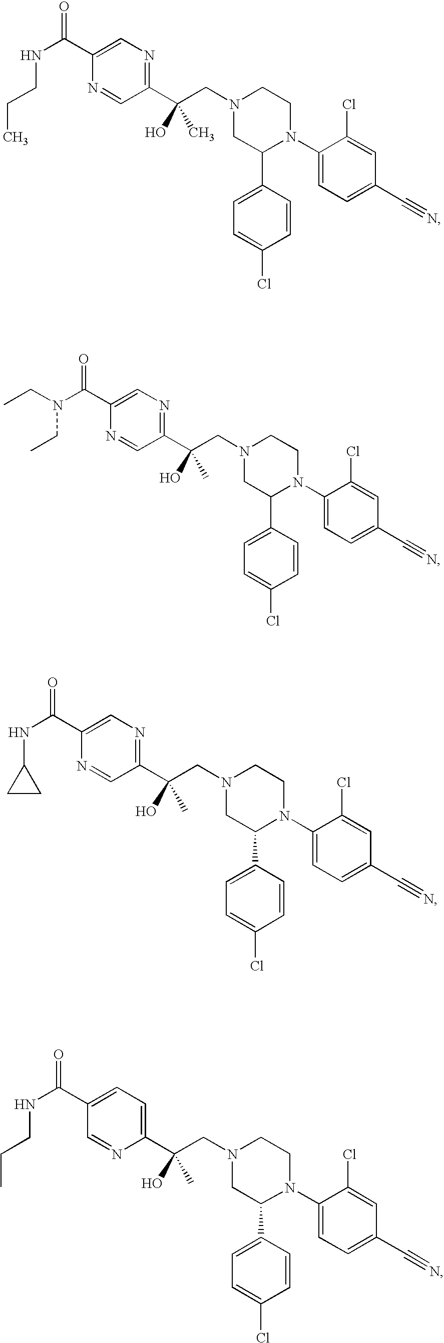

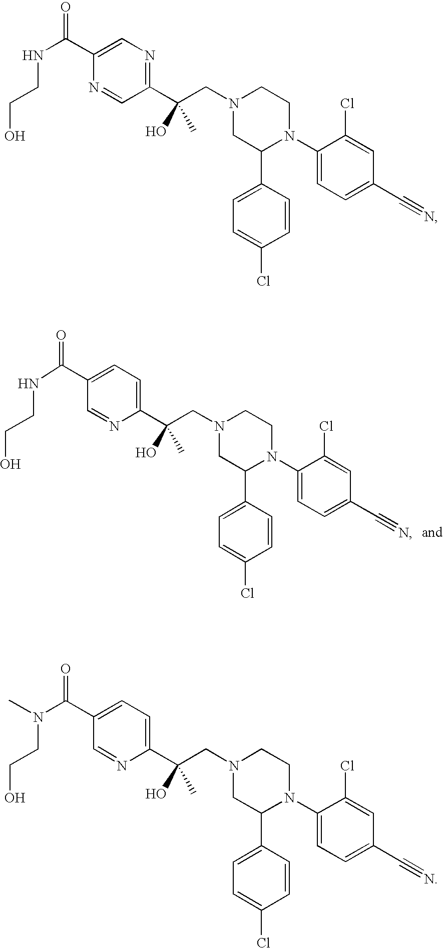

38. A composition comprising: at least one compound according to claim 1, or a pharmaceutically acceptable salt, thereof; and at least one pharmaceutically acceptable carrier.

39. A composition comprising: at least one compound claim 1, or a pharmaceutically acceptable salt, thereof: and at least one additional active agent other than a compound of claim 1.

40-46. (canceled)

47. A method of treating, reducing, or ameliorating a condition or disease selected from psychic disorders, anxiety, schizophrenia, depression, abuse of psychotropes, substance abuse, substance dependency, alcohol dependency, nicotine dependency, neuropathies, migraine, stress, epilepsy, dyskinesias, Parkinson's disease, amnesia, senile dementia. Alzheimer's disease, eating disorders, type n diabetes, gastrointestinal diseases, vomiting, diarrhea, urinary disorders, infertility disorders, inflammation, infection, cancer, neuroinflammation, atherosclerosis, Guillain-Barr syndrome, viral encephalitis, cerebral vascular incidents, and cranial trauma in a patient in need thereof, comprising: administering to said patient in need thereof an effective amount of a compound of claim 1, or a pharmaceutically acceptable salt, thereof.

48. A method of treating, reducing, or ameliorating a condition or disease selected from metabolic syndrome, obesity, waist circumference, abdominal girth, type II diabetes, insulin resistance, hepatic lipidosis, fatty liver disease, neuroinflammatory disorders, cognitive disorders, psychosis, addictive behavior, gastrointestinal disorders, and cardiovascular conditions, in a patient in need thereof, comprising: administering to said patient in need thereof an effective amount of at least one compound according to claim 1, or a pharmaceutically acceptable salt, thereof.

49. The method of claim 48, wherein said condition or disease is selected from metabolic syndrome, obesity, waist circumference, abdominal girth, type II diabetes, hepatic lipidosis, and fatty liver disease.

50. A method of reducing body condition score in a patient in need thereof, comprising administering to said patient in need thereof an effective amount of at least one compound according to claim 1, or a pharmaceutically acceptable salt, thereof.

51. A method of treating, reducing, or ameliorating a condition or disease selected from psychic disorders, anxiety, schizophrenia, depression, abuse of psychotropes, substance abuse, substance dependency, alcohol dependency, nicotine dependency, neuropathies, migraine, stress, epilepsy, dyskinesias, Parkinson's disease, amnesia, senile dementia, Alzheimer's disease, eating disorders, type II diabetes, gastrointestinal diseases, vomiting, diarrhea, urinary disorders, infertility disorders, inflammation, infection, cancer, neuroinflammation, atherosclerosis, Guillain-Barr syndrome, viral encephalitis, cerebral vascular incidents, and cranial trauma in a patient in need thereof, comprising: administering to said patient in need thereof an effective amount of a composition according to claim 39.

52. A method of treating, reducing, or ameliorating a condition or disease selected from metabolic syndrome, obesity, waist circumference, abdominal girth, type II diabetes, insulin resistance, hepatic lipidosis, fatty liver disease, neuroinflammatory disorders, cognitive disorders, psychosis, addictive behavior, gastrointestinal disorders, and cardiovascular conditions, in a patient in need thereof, comprising: administering to a patient in need thereof an effective amount of a composition according to claim 39.

53. A method of treating, reducing, or ameliorating a condition or disease selected from metabolic syndrome, obesity, waist circumference, abdominal girth, type II diabetes, hepatic lipidosis, and fatty liver disease, comprising administering to a patient in need thereof an effective amount of a composition of claim 39.

54. A method of reducing body condition score in a patient in need thereof, comprising: administering to said patient in need thereof an effective amount of a composition according to claim 39.

55. A method of partitioning energy of an animal away from fat deposition toward protein accretion, comprising administering to said animal an effective amount of at least one compound according to claim 1, or a pharmaceutically acceptable salt, thereof.

56. A method of partitioning energy of an animal away from fat deposition toward protein accretion, comprising: administering to said animal an effective amount of a composition according to claim 39.

57. A composition comprising: at least one compound according to claim 37, or a pharmaceutically acceptable salt thereof; and at least one pharmaceutically acceptable carrier.
